SEQUENCE LISTING (109 sequences)

SEQ ID NO: 1 - protein, 8 aa, Artificial Sequence
  Description of Artificial Sequence Synthetic peptide
CWKKKKKK

SEQ ID NO: 2 - protein, 12 aa, Artificial Sequence
  Description of Artificial Sequence Synthetic peptide
KKKKKKKKKKKK

SEQ ID NO: 3 - protein, 5 aa, Artificial Sequence
  Description of Artificial Sequence Synthetic peptide
CWKKK

SEQ ID NO: 4 - protein, 6 aa, Artificial Sequence
  Description of Artificial Sequence Synthetic peptide
CWKKKK

SEQ ID NO: 5 - protein, 7 aa, Artificial Sequence
  Description of Artificial Sequence Synthetic peptide
CWKKKKK

SEQ ID NO: 6 - protein, 5 aa, Artificial Sequence
  Description of Artificial Sequence Synthetic peptide
CKKKK

SEQ ID NO: 7 - protein, 8 aa, Artificial Sequence
  Description of Artificial Sequence Synthetic peptide
KRKRKRKR

SEQ ID NO: 8 - protein, 8 aa, Artificial Sequence
  Description of Artificial Sequence Synthetic peptide
KRKRKRKR

SEQ ID NO: 9 - protein, 13 aa, Artificial Sequence
  Description of Artificial Sequence Synthetic peptide
KKKKKKKKKKKKC

SEQ ID NO: 10 - protein, 210 aa, Artificial Sequence
  Description of Artificial Sequence Synthetic polypeptide
CKKXKKCCKKXKKCCKKXKKCCKKXKKCCKKXKKCCKKXKKCCKKXKKCCKKXKKCCKKXKKCCKKXKKCCKKXKKCCKKXKKCCKKXKKCCKKXKKCCKKXKKCCKKXKKCCKKXKKCCKKXKKCCKKXKKCCKKXKKCCKKXKKCCKKXKKCCKKXKKCCKKXKKCCKKXKKCCKKXKKCCKKXKKCCKKXKKCCKKXKKCCKKXKKC

SEQ ID NO: 11 - protein, 9 aa, Artificial Sequence
  Description of Artificial Sequence Synthetic peptide
KRKRKRKRC

SEQ ID NO: 12 - protein, 10 aa, Artificial Sequence
  Description of Artificial Sequence Synthetic peptide
KRKRKRKRCC

SEQ ID NO: 13 - protein, 19 aa, Artificial Sequence
  Description of Artificial Sequence Synthetic peptide
WKKKKKKKKKKKKKKKKKK

SEQ ID NO: 14 - protein, 5 aa, Artificial Sequence
  Description of Artificial Sequence Synthetic peptide
CWKKK

SEQ ID NO: 15 - protein, 6 aa, Artificial Sequence
  Description of Artificial Sequence Synthetic peptide
CWKKKK

SEQ ID NO: 16 - protein, 7 aa, Artificial Sequence
  Description of Artificial Sequence Synthetic peptide
CWKKKKK

SEQ ID NO: 17 - protein, 5 aa, Artificial Sequence
  Description of Artificial Sequence Synthetic peptide
CWKKK

SEQ ID NO: 18 - protein, 6 aa, Artificial Sequence
  Description of Artificial Sequence Synthetic peptide
CWKKKK

SEQ ID NO: 19 - protein, 7 aa, Artificial Sequence
  Description of Artificial Sequence Synthetic peptide
CWKKKKK

SEQ ID NO: 20 - protein, 4 aa, Artificial Sequence
  Description of Artificial Sequence Synthetic peptide
CKKK

SEQ ID NO: 21 - protein, 5 aa, Artificial Sequence
  Description of Artificial Sequence Synthetic peptide
CKKKK

SEQ ID NO: 22 - protein, 6 aa, Artificial Sequence
  Description of Artificial Sequence Synthetic peptide
CKKKKK

SEQ ID NO: 23 - protein, 9 aa, Artificial Sequence
  Description of Artificial Sequence Synthetic peptide
CKKKKKKKK

SEQ ID NO: 24 - protein, 8 aa, Artificial Sequence
  Description of Artificial Sequence Synthetic peptide
KRKRKRKR

SEQ ID NO: 25 - protein, 8 aa, Artificial Sequence
  Description of Artificial Sequence Synthetic peptide
KRKRKRKR

SEQ ID NO: 26 - protein, 8 aa, Artificial Sequence
  Description of Artificial Sequence Synthetic peptide
KRKRKRKR

SEQ ID NO: 27 - protein, 9 aa, Artificial Sequence
  Description of Artificial Sequence Synthetic peptide
KRKRKRKRC

SEQ ID NO: 28 - protein, 8 aa, Artificial Sequence
  Description of Artificial Sequence Synthetic peptide
KRKRKRKR

SEQ ID NO: 29 - protein, 8 aa, Artificial Sequence
  Description of Artificial Sequence Synthetic peptide
KKKKKKKK

SEQ ID NO: 30 - protein, 8 aa, Artificial Sequence
  Description of Artificial Sequence Synthetic peptide
KLKLKLKL

SEQ ID NO: 31 - protein, 8 aa, Artificial Sequence
  Description of Artificial Sequence Synthetic peptide
KEKEKEKE

SEQ ID NO: 32 - protein, 4 aa, Artificial Sequence
  Description of Artificial Sequence Synthetic peptide
KKKK

SEQ ID NO: 33 - protein, 12 aa, Artificial Sequence
  Description of Artificial Sequence Synthetic peptide
KKKKKKKKKKKK

SEQ ID NO: 34 - protein, 20 aa, Artificial Sequence
  Description of Artificial Sequence Synthetic peptide
CWKKKKKKKKKKKKKKKKKK

SEQ ID NO: 35 - protein, 9 aa, Artificial Sequence
  Description of Artificial Sequence Synthetic peptide
KRKRKRKRC

SEQ ID NO: 36 - protein, 9 aa, Artificial Sequence
  Description of Artificial Sequence Synthetic peptide
KKKKKKKKC

SEQ ID NO: 37 - protein, 9 aa, Artificial Sequence
  Description of Artificial Sequence Synthetic peptide
KLKLKLKLC

SEQ ID NO: 38 - protein, 9 aa, Artificial Sequence
  Description of Artificial Sequence Synthetic peptide
KEKEKEKEC

SEQ ID NO: 39 - protein, 13 aa, Artificial Sequence
  Description of Artificial Sequence Synthetic peptide
KKKKKKKKKKKKC

SEQ ID NO: 40 - protein, 5 aa, Artificial Sequence
  Description of Artificial Sequence Synthetic peptide
KKKKC

SEQ ID NO: 41 - protein, 13 aa, Artificial Sequence
  Description of Artificial Sequence Synthetic peptide
KKKKKKKKKKKKC

SEQ ID NO: 42 - protein, 12 aa, Artificial Sequence
  Description of Artificial Sequence Synthetic peptide
KKKKKKKKKKKK

SEQ ID NO: 43 - protein, 4 aa, Artificial Sequence
  Description of Artificial Sequence Synthetic peptide
CKKC

SEQ ID NO: 44 - protein, 6 aa, Artificial Sequence
  Description of Artificial Sequence Synthetic peptide
KKKKKK

SEQ ID NO: 45 - protein, 10 aa, Artificial Sequence
  Description of Artificial Sequence Synthetic peptide
GGPKKKRKVG

SEQ ID NO: 46 - protein, 10 aa, Artificial Sequence
  Description of Artificial Sequence Synthetic peptide
CGPKKKRKVG

SEQ ID NO: 47 - protein, 10 aa, Artificial Sequence
  Description of Artificial Sequence Synthetic peptide
GGPKTKRKVG

SEQ ID NO: 48 - protein, 10 aa, Artificial Sequence
  Description of Artificial Sequence Synthetic peptide
CGPKTKRKVG

SEQ ID NO: 49 - protein, 10 aa, Artificial Sequence
  Description of Artificial Sequence Synthetic peptide
GGPKTKRKVG

SEQ ID NO: 50 - protein, 26 aa, Artificial Sequence
  Description of Artificial Sequence Synthetic peptide
GIGAVLKVLTTGLPALISWIKRKRQQ

SEQ ID NO: 51 - protein, 4 aa, Artificial Sequence
  Description of Artificial Sequence Synthetic peptide
CWKK

SEQ ID NO: 52 - protein, 30 aa, Artificial Sequence
  Description of Artificial Sequence Synthetic polypeptide
CWKKGIGAVLKVLTTGLPALISLIKRKRQQ

SEQ ID NO: 53 - protein, 27 aa, Artificial Sequence
  Description of Artificial Sequence Synthetic peptide
WGIGAVLKVLTTGLPALISLIKKKKQQ

SEQ ID NO: 54 - protein, 28 aa, Artificial Sequence
  Description of Artificial Sequence Synthetic peptide
CWGIGAVLKVLTTGLPALISLIKKKKQQ

SEQ ID NO: 55 - protein, 28 aa, Artificial Sequence
  Description of Artificial Sequence Synthetic peptide
GWGIGAVLKVLTTGLPALISLIKKKKQQ

SEQ ID NO: 56 - protein, 37 aa, Artificial Sequence
  Description of Artificial Sequence Synthetic polypeptide
CWGIGAVLKVLTTGLPALISLIKKKKQQGPKKKRKVG

SEQ ID NO: 57 - protein, 37 aa, Artificial Sequence
  Description of Artificial Sequence Synthetic polypeptide
CGPKKKRKVGWGIGAVLKVLTTGLPALISLIKKKKQQ

SEQ ID NO: 58 - protein, 36 aa, Artificial Sequence
  Description of Artificial Sequence Synthetic polypeptide
CGPKKKRKVWGIGAVLKVLTTGLPALISLIKKKKQQ

SEQ ID NO: 59 - protein, 13 aa, Artificial Sequence
  Description of Artificial Sequence Synthetic peptide
CSSAWWSYWPPVA

SEQ ID NO: 60 - protein, 13 aa, Artificial Sequence
  Description of Artificial Sequence Synthetic peptide
GSSAWWSYWPPVA

SEQ ID NO: 61 - protein, 23 aa, Artificial Sequence
  Description of Artificial Sequence Synthetic peptide
CSSAWWSYWPPVAGGPKKKRKVG

SEQ ID NO: 62 - protein, 23 aa, Artificial Sequence
  Description of Artificial Sequence Synthetic peptide
CGGPKKKRKVGSSAWWSYWPPVA

SEQ ID NO: 63 - protein, 20 aa, Artificial Sequence
  Description of Artificial Sequence Synthetic peptide
CGLFEALLELLESLWELLEA

SEQ ID NO: 64 - protein, 43 aa, Artificial Sequence
  Description of Artificial Sequence Synthetic peptide
KXKXKXKXKXKXCCWKKGIGAVLKVLTTGLPALISLIKRKRQQ

SEQ ID NO: 65 - protein, 9 aa, Artificial Sequence
  Description of Artificial Sequence Synthetic peptide
KXKXKXKXC

SEQ ID NO: 66 - protein, 4 aa, Artificial Sequence
  Description of Artificial Sequence Synthetic peptide
KALA

SEQ ID NO: 67 - protein, 30 aa, Artificial Sequence
  Description of Artificial Sequence Synthetic polypeptide
GWKKGIGAVLKVLTTGLPALISLIKRKRQQ

SEQ ID NO: 68 - protein, 8 aa, Artificial Sequence
  Description of Artificial Sequence Synthetic peptide
KRKRKRKR

SEQ ID NO: 69 - protein, 5 aa, Artificial Sequence
  Description of Artificial Sequence Synthetic peptide
KRKRC

SEQ ID NO: 70 - protein, 7 aa, Artificial Sequence
  Description of Artificial Sequence Synthetic peptide
KRKRKRC

SEQ ID NO: 71 - protein, 9 aa, Artificial Sequence
  Description of Artificial Sequence Synthetic peptide
KKKKKKKKC

SEQ ID NO: 72 - protein, 9 aa, Artificial Sequence
  Description of Artificial Sequence Synthetic peptide
KLKLKLKLC

SEQ ID NO: 73 - protein, 10 aa, Artificial Sequence
  Description of Artificial Sequence Synthetic peptide
WKRKRKRKRC

SEQ ID NO: 74 - protein, 30 aa, Artificial Sequence; identical to SEQ ID NO: 52
  Description of Artificial Sequence Synthetic polypeptide

SEQ ID NO: 75 - protein, 30 aa, Artificial Sequence; identical to SEQ ID NO: 52
  Description of Artificial Sequence Synthetic polypeptide

SEQ ID NO: 76 - protein, 35 aa, Artificial Sequence
  Description of Artificial Sequence Synthetic polypeptide
KRKRCCWKKGIGAVLKVLTTGLPALISLIKRKRQQ

SEQ ID NO: 77 - protein, 37 aa, Artificial Sequence
  Description of Artificial Sequence Synthetic polypeptide
KRKRKRCCWKKGIGAVLKVLTTGLPALISLIKRKRQQ

SEQ ID NO: 78 - protein, 39 aa, Artificial Sequence
  Description of Artificial Sequence Synthetic polypeptide
KRKRKRKRCCWKKGIGAVLKVLTTGLPALISLIKRKRQQ

SEQ ID NO: 79 - protein, 40 aa, Artificial Sequence
  Description of Artificial Sequence Synthetic polypeptide
WKRKRKRKRCCWKKGIGAVLKVLTTGLPALISLIKRKRQQ

SEQ ID NO: 80 - protein, 39 aa, Artificial Sequence
  Description of Artificial Sequence Synthetic polypeptide
KKKKKKKKCCWKKGIGAVLKVLTTGLPALISLIKRKRQQ

SEQ ID NO: 81 - protein, 39 aa, Artificial Sequence
  Description of Artificial Sequence Synthetic polypeptide
KLKLKLKLCCWKKGIGAVLKVLTTGLPALISLIKRKRQQ

SEQ ID NO: 82 - protein, 20 aa, Artificial Sequence
  Description of Artificial Sequence Synthetic peptide
CWKKKKKKKKKKKKKKKKKK

SEQ ID NO: 83 - protein, 12 aa, Artificial Sequence
  Description of Artificial Sequence Synthetic peptide
KKKKKKKKKKKK

SEQ ID NO: 84 - protein, 12 aa, Artificial Sequence
  Description of Artificial Sequence Synthetic peptide
KKKKKKKKKKKK

SEQ ID NO: 85 - protein, 18 aa, Artificial Sequence
  Description of Artificial Sequence Synthetic peptide
KKKKKKKKKKKKKKKKKK

SEQ ID NO: 86 - protein, 5 aa, Artificial Sequence
  Description of Artificial Sequence Synthetic peptide
CKKKK

SEQ ID NO: 87 - protein, 9 aa, Artificial Sequence
  Description of Artificial Sequence Synthetic peptide
KRKRKRKRC

SEQ ID NO: 88 - protein, 9 aa, Artificial Sequence
  Description of Artificial Sequence Synthetic peptide
KXKXKXKXC

SEQ ID NO: 89 - protein, 12 aa, Artificial Sequence
  Description of Artificial Sequence Synthetic peptide
KXKXKXKXKXKX

SEQ ID NO: 90 - protein, 9 aa, Artificial Sequence
  Description of Artificial Sequence Synthetic peptide
KXKXKXKXC

SEQ ID NO: 91 - protein, 5 aa, Artificial Sequence
  Description of Artificial Sequence Synthetic peptide
KKKKC

SEQ ID NO: 92 - protein, 13 aa, Artificial Sequence
  Description of Artificial Sequence Synthetic peptide
KKKKKKKKKKKKC

SEQ ID NO: 93 - protein, 9 aa, Artificial Sequence
  Description of Artificial Sequence Synthetic peptide
KEKEKEKEC

SEQ ID NO: 94 - protein, 4 aa, Artificial Sequence
  Description of Artificial Sequence Synthetic peptide
KKKK

SEQ ID NO: 95 - protein, 8 aa, Artificial Sequence
  Description of Artificial Sequence Synthetic peptide
KKKKKKKK

SEQ ID NO: 96 - protein, 12 aa, Artificial Sequence
  Description of Artificial Sequence Synthetic peptide
KKKKKKKKKKKK

SEQ ID NO: 97 - protein, 8 aa, Artificial Sequence
  Description of Artificial Sequence Synthetic peptide
KLKLKLKL

SEQ ID NO: 98 - protein, 8 aa, Artificial Sequence
  Description of Artificial Sequence Synthetic peptide
KEKEKEKE

SEQ ID NO: 99 - protein, 8 aa, Artificial Sequence
  Description of Artificial Sequence Synthetic peptide
KXKXKXKX

SEQ ID NO: 100 - protein, 12 aa, Artificial Sequence
  Description of Artificial Sequence Synthetic peptide
KKKKKKKKKKKK

SEQ ID NO: 101 - protein, 12 aa, Artificial Sequence
  Description of Artificial Sequence Synthetic peptide
KKKKKKKKKKKK

SEQ ID NO: 102 - protein, 13 aa, Artificial Sequence
  Description of Artificial Sequence Synthetic peptide
CKKKKKKKKKKKK

SEQ ID NO: 103 - protein, 5 aa, Artificial Sequence
  Description of Artificial Sequence Synthetic peptide
CKKKC

SEQ ID NO: 104 - protein, 5 aa, Artificial Sequence
  Description of Artificial Sequence Synthetic peptide
CKRKC

SEQ ID NO: 105 - protein, 5 aa, Artificial Sequence
  Description of Artificial Sequence Synthetic peptide
CKLKC

SEQ ID NO: 106 - protein, 5 aa, Artificial Sequence
  Description of Artificial Sequence Synthetic peptide
CKFKC

SEQ ID NO: 107 - protein, 8 aa, Artificial Sequence
  Description of Artificial Sequence Synthetic peptide
KKKKKKWC

SEQ ID NO: 108 - protein, 80 aa, Artificial Sequence
  Description of Artificial Sequence Synthetic polypeptide
XCXXXXXXXCXXXXXXXCXXXXXXXCXXXXXXXCXXXXXXXCXXXXXXXCXXXXXXXCXXXXXXXCXXXXXXXCXXXXXX

SEQ ID NO: 109 - protein, 80 aa, Artificial Sequence
  Description of Artificial Sequence Synthetic polypeptide
XXXXXXCXXXXXXXCXXXXXXXCXXXXXXXCXXXXXXXCXXXXXXXCXXXXXXXCXXXXXXXCXXXXXXXCXXXXXXXCX

(19) United States
(12) Patent Application Publication
(10) Pub. No.: US 2012/0035320 A1
(43) Pub. Date: Feb. 9, 2012

(54) POLYACRIDINE NUCLEIC ACID DELIVERY PEPTIDE COMPLEXES

(75) Inventors: Kevin G. Rice, Iowa City, IA (US); Nicholas J. Baumhover, Iowa City, IA (US); Christian A. Fernandez, Iowa City, IA (US); Kevin Anderson, Iowa City, IA (US); Mark D. Ericson, Iowa City, IA (US); Jason T. Duskey, Iowa City, IA (US); Koby Kizzire, Iowa City, IA (US); Sanjib Khargharia, Iowa City, IA (US); Samuel T. Crowley, Iowa City, IA (US)

(73) Assignee: University of Iowa Research Foundation, Iowa City, IA (US)

(21) Appl. No.: 13/079,580

(22) Filed: Apr. 4, 2011

Related U.S. Application Data
(60) Provisional application No. 61/320,726, filed on Apr. 3, 2010.

(51) Int. Cl. C08G 65/333; C07K 4/00; C07K 14/00; C07K 19/00; C12N 9/96

(52) U.S. Cl. 525/54.1; 530/300; 530/323; 530/322; 530/367; 435/188; 530/358

(57) ABSTRACT

The present invention provides nucleic acid delivery polyplex complexes and anionic open polyplexes comprising a nucleic acid molecule reversibly bound to one or more of nucleic acid delivery polyplex complexes.

### RELATED APPLICATION

This application claims priority to U.S. Provisional Patent Application No. 61/320,726, filed Apr. 3, 2010, the entirety of which is incorporated herein by reference.

### GOVERNMENT FUNDING

The invention described herein was made with government support under Grant Number DK066212, T32 GM067795, and RO1 GM087653 awarded by the National Institutes of Health. The United States Government has certain rights in this invention.

### SEQUENCE LISTING

The instant application contains a Sequence Listing which has been submitted in ASCII format via EFS-Web and is hereby incorporated by reference in its entirety. Said ASCII copy, created on Jun. 29, 2011, is named 17023104.txt and is 73,787 bytes in size.

### BACKGROUND OF THE INVENTION

Researchers have investigated various gene therapy methods in order to develop techniques that could deliver a therapeutic gene (transgene) to selected cells in which proper gene expression is desired. Such methods need to protect the transgene against degradation by nucleases in extracellular matrices, bring the transgene across the plasma membrane and into the nucleus of target cells, and have no detrimental effects.

Scientists have successfully used viral vectors to mediate gene transfer with high efficiency and the possibility of long-term gene expression, but safety concerns have not yet been adequately overcome. Other practical issues with respect to viral vector gene transfer relate to size limitations (i.e., the size of the transgene that recombinant viruses can carry) and difficulties in the production of viral vectors. Many studies have investigated methods of non-viral gene delivery using physical (carrier-free gene delivery) and chemical approaches (synthetic vector-based gene delivery). Chemical approaches use synthetic or naturally occurring compounds as carriers to deliver the transgene into cells. Although significant progress has been made in the basic science and applications of various non-viral gene delivery systems, the majority of nonviral approaches are still much less efficient than viral vectors, especially for in vivo gene delivery.

### SUMMARY OF THE INVENTION

The present invention provides a nucleic acid delivery peptide complex, comprising

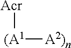

operably linked to

Cys-L¹-R¹

wherein Acr is an acridine; A¹ is Lysine, Arginine, Leucine or Glutamic acid; A² is 1 to 6 amino acids; Cys is cysteine, wherein the Cys is bound to L¹ at the amino-terminus, the carboxy-terminus or an amino acid side-chain; and n is 1 to 10; wherein L¹ is a linker; wherein R¹ is one or more of a peptide, polyethylene glycol (PEG), a carbohydrate, biotin or avidin, and/or is a hydrogen; and wherein the nucleic acid delivery peptide reversibly binds to nucleic acid.

As used herein, the Cys-L¹-R¹ moiety may be linked to the acridine-peptide moiety at the amino-terminus of the acridine-peptide moiety, at the carboxy-terminus of the acridine-peptide moiety, or at one of the side chains of the amino acids that make up the of the acridine-peptide moiety. For example the structure of the peptide may be as follows in Formula Ia, I b, Ic or Id:

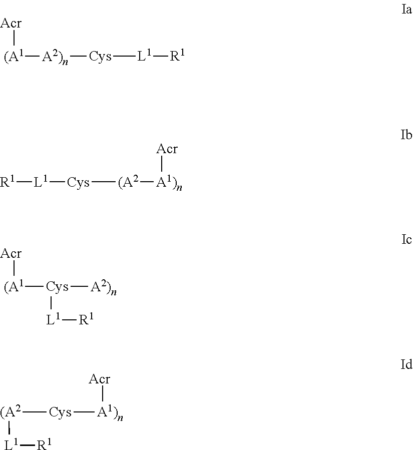

In certain embodiments, L¹ is —S—; a disulfide linkage; a maleimide linkage; an amide linkage, ester linkage, ether linkage, or a thiol ether linkage, hydrazone linkage.

In certain embodiments, R¹ is a carbohydrate, such as a targeting ligand. In certain embodiments, the carbohydrate is a triantennary N-glycan, such as a high-mannose N-glycan. In certain embodiments, the carbohydrate is Man₉GlcNAc₂.

In certain embodiments, R¹ is a peptide, such as a nuclear localization signal (NLS) peptide, a fusogenic peptide, a targeting peptide, an NLS-fusogenic peptide, or an enzyme. In certain embodiments, the R¹ peptide is NLS (SEQ ID NO: 45); Cys NLS (SEQ ID NO: 46); Control NLS (SEQ ID NO: 47); Cys-Control NLS (SEQ ID NO: 48); Mal-Gly-Control-NLS (SEQ ID NO: 49); Melittin (natural melittin) (SEQ ID NO: 50); Cys-melittin (SEQ ID NO: 51); K-Melittin (SEQ ID NO: 52); Cys-K-Melittin (SEQ ID NO: 53); Mal-Gly-K-Melittin (maleimide) (SEQ ID NO: 54); Cys-K-Melittin-NLS (SEQ ID NO: 55); Cys-NLS-K-Melittin (SEQ ID NO: 56); Modified Cys-NLS-Melittin (SEQ ID NO: 57); PC4—NLS Analogue Cys-PC4 (SEQ ID NO: 58); PC4—NLS Analogue Maleimide-PC4 (SEQ ID NO: 59); PC4—NLS Analogue Cys-PC4—NLS (SEQ ID NO: 60); PC4—NLS Analogue Cys-NLS-PC4 (SEQ ID NO: 61); Cys-JTS (SEQ ID NO: 62) or Octreotide.

In certain embodiments, R¹ is polyethylene glycol (PEG). In certain embodiments, the PEG is between 500 g/mol and 20,000 g/mol, such as 10,000 g/mol, 5,000 g/mol, 2,000 g/mol or 500 g/mol.

In certain embodiments, L¹ is a maleimide linkage. In certain embodiments, L¹ is a disulfide linkage.

In certain embodiments, A¹ is Lys having an c-amine, and Acr is linked to the ε-amine of Lys.

In certain embodiments, A² is one or more of any of the natural 20 amino acids or any other unnatural amino acids. In certain embodiments, A² is one or more of Arg, Lys, Glu or Leu. In certain embodiments, A² is 1 to 6 Lys residues. In certain embodiments, A² is 1 to 6 Arg residues. In certain embodiments, A² is 1 to 6 Glu residues. In certain embodiments, A² is 1 to 6 Leu residues. In certain embodiments, A² is 1 to 4 amino acids. In certain embodiments, A² is 1 or 2 amino acids.

In certain embodiments, n is 1 to 8. In certain embodiments, n is 3 to 6.

In certain embodiments, Acr is a substituted acridine. In certain embodiments, Acr is a 9-aminoacridine, such as 9-aminoacridine is 9-alkylaminoacridine, 9-arylaminoacridine, 9-(N-carboxamido) acridine or 9-ureidoacridine.

The present invention provides a nucleic acid delivery peptide complex, comprising Formula II:

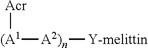

wherein
Acr is acridine;
Y is —S—S— or maleimide;
A¹ is Arginine, Leucine or Lysine;
A² is Arginine, Leucine or Lysine;
n is 2 to 6;
wherein the nucleic acid delivery peptide complex binds to nucleic acid.

In certain embodiments, the melittin is Melittin (natural melittin); Cys-melittin (SEQ ID NO: 52); K-Melittin (SEQ ID NO: 53); Cys-K-Melittin (SEQ ID NO: 54); Mal-Gly-K-Melittin (maleimide) (SEQ ID NO: 55); Cys-K-Melittin-NLS (SEQ ID NO: 56); Cys-NLS-K-Melittin (SEQ ID NO: 57); or Modified Cys-NLS-Melittin (SEQ ID NO: 58). In certain embodiments, A¹ is Arginine and A² is Arginine.

The present invention provides a nucleic acid delivery peptide complex, comprising Formula III:

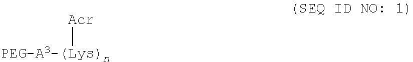

wherein Acr is acridine; A³ is Cys or Cys-Trp; and n is 1 to 6; wherein the nucleic acid delivery peptide complex binds to nucleic acid.

In certain embodiments, n is 5. In certain embodiments, PEG is between 500 g/mol and 20,000 g/mol.

The present invention provides a nucleic acid delivery peptide complex, comprising Formula IV:

(SEQ ID NO: 2)
Man9-L³-(Acr-Lys)ₙ

wherein L³ is a linker, and n is 4-6. In certain embodiments, the linker is an acetamide linkage or a maleimide linkage. In certain embodiments, the compound is Formula IVa:

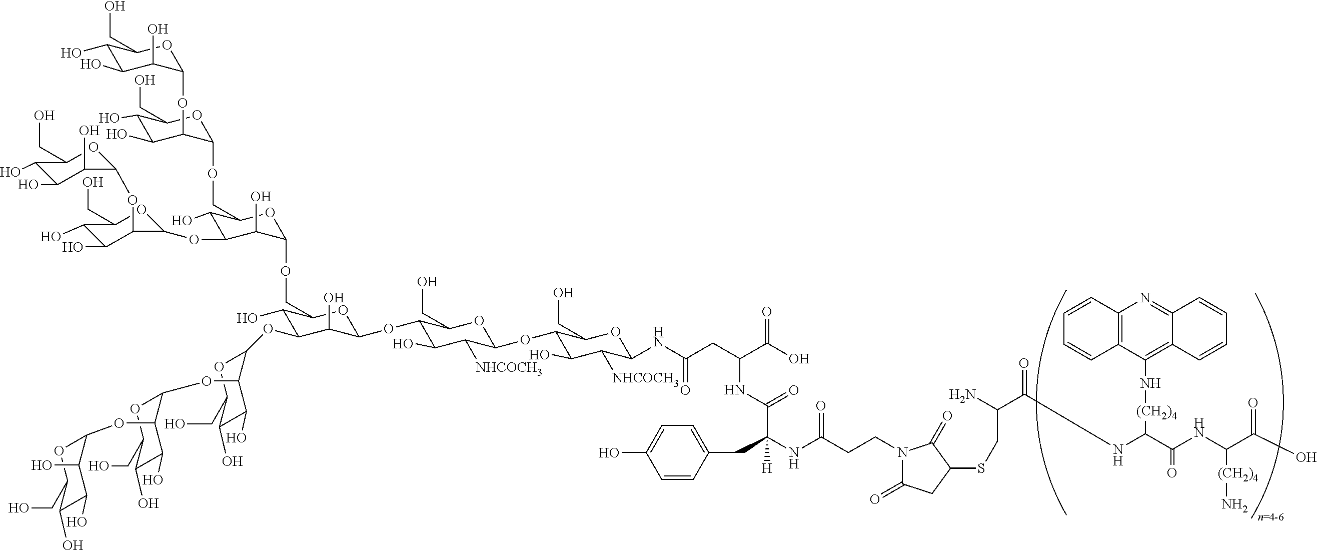

In certain embodiments the compound is Formula IVb:

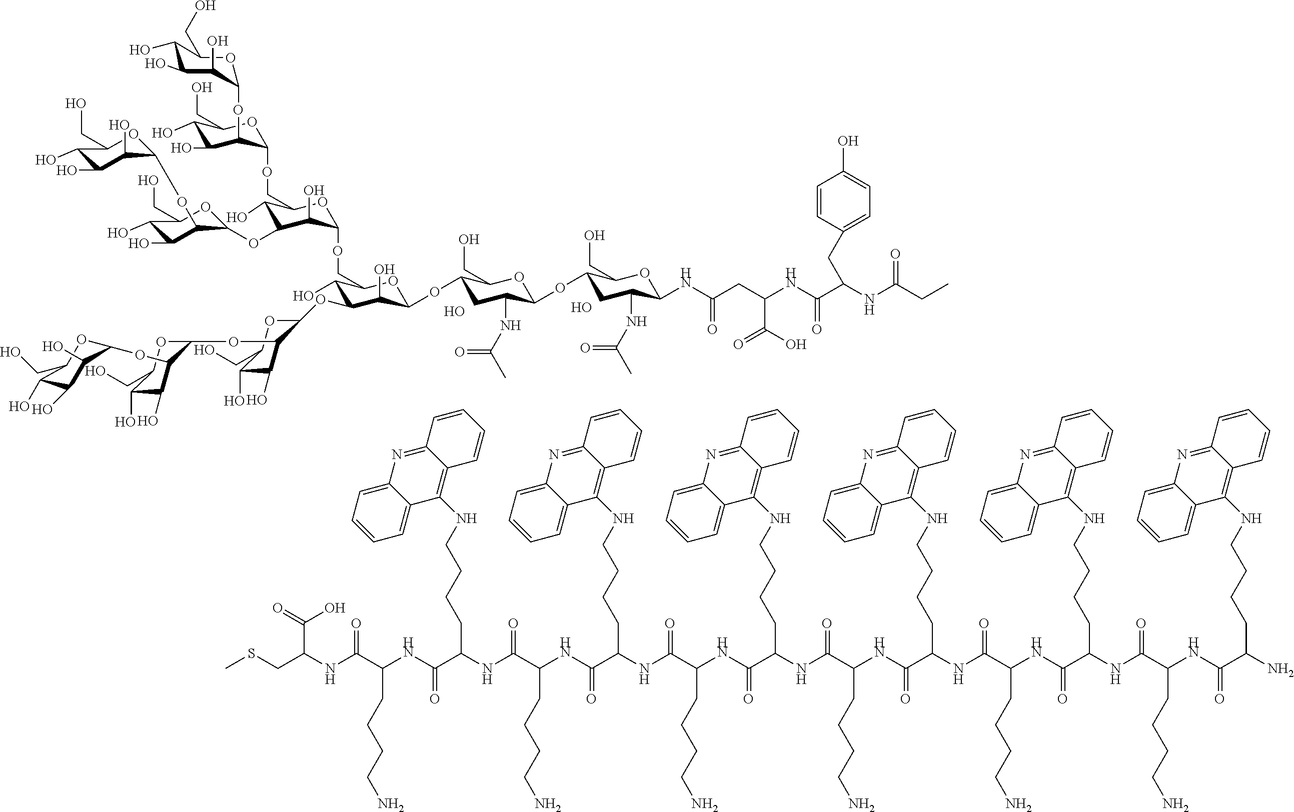

The present invention provides a peptide complex of poly(Acr-Arg)₄-SS-melittin (SEQ ID NO: 78); poly(Acr-Arg)₄-Mal-melittin (SEQ ID NOS 11 & 67, respectively); PEG-Cys-Trp-(Lys(Acr))₃ (SEQ ID NO: 3); PEG-Cys-Trp-(Lys(Acr))₄ (SEQ ID NO: 4); PEG-Cys-Trp-(Lys(Acr))₅ (SEQ ID NO: 5); Man9-Mal-Cys-(Lys(Acr))₄ (SEQ ID NO: 6); poly(Acr-Arg)₄-SS-PEG (SEQ ID NO: 7); poly(Acr-Arg)₄-Mal-PEG (SEQ ID NO: 8).

The present invention provides a nucleic acid delivery complex comprising a nucleic acid molecule reversibly bound to one or more of the peptide complexes described above. In certain embodiments, the nucleic acid is an RNA molecule or a DNA molecule, such as a double-stranded RNA or DNA molecule.

The present invention provides an anionic open polyplex comprising a nucleic acid molecule reversibly bound to a nucleic acid delivery peptide complex comprising

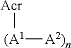

operably linked to

Cys-L¹-R¹

wherein Acr is an acridine; A¹ is Lysine, Arginine, Leucine or Glutamic acid; A² is 1 to 6 amino acids; Cys is cysteine, wherein the Cys is bound to L¹ at the amino-terminus, the carboxy-terminus or an amino acid side-chain; and n is 1 to 10; wherein L¹ is a linker; wherein R¹ is one or more of a peptide, polyethylene glycol (PEG), a carbohydrate, biotin or avidin and/or is a hydrogen; and wherein the nucleic acid delivery peptide complex reversibly binds to nucleic acid, wherein the anionic open polyplex is stable against DNase or RNase digestions. As used herein, the term “stable against DNase or RNase digestions” means that at least 60% of the original anionic open polyplex remains after incubation at 37° C. for 1 hour. Anionic open polyplex is a DNA or RNA peptide complex in which the DNA or RNA remains stable for 1 hour when reacted with up to 60 milli-units (International Units) of DNase or RNase.

The present invention provides an anionic open polyplex comprising a nucleic acid molecule reversibly bound to a nucleic acid delivery peptide complex comprising Formula Ia, Ib, Ic, Id, II, III, IV, IVa or IVb wherein the nucleic acid delivery peptide complex reversibly binds to nucleic acid, wherein the anionic open polyplex is stable against DNase or RNase digestions.

In certain embodiments, the anionic open polyplex is electronegatively charged. In certain embodiments, the anionic open polyplex has a diameter of 100 nm or greater, where the definition of diameter is the longest end-to-end distance spanning the polyplex as determined by atomic force microscopy. In certain embodiments, the anionic open polyplex possesses a shape that is similar to uncomplexed DNA or RNA, i.e., where the shape is a random coil. In certain embodiments, the anionic open polyplex has a long circulatory half-life, such as, for example, a beta pharmacokinetic half-life of 60 minutes or greater.

The present invention provides a composition including a polyplex of as described above and a physiologically acceptable carrier.

The present invention provides a therapeutic method for preventing or treating a pathological condition or symptom in a mammal, such as a human, by administering to a mammal in need of such therapy, an effective amount of nucleic acid polyplex or nucleic acid delivery complex as described above.

The present invention provides a nucleic acid polyplex or nucleic acid delivery complex for use in medical therapy.

The present invention provides the use of nucleic acid polyplex or nucleic acid delivery complex for the manufacture of a medicament useful for the treatment of a pathological condition or symptom in a mammal.

The present invention provides a nucleic acid delivery peptide complex comprising:
(a) a compound of Formula V:

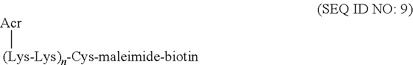

wherein n is 4-6;
(b) a biotinylated phospholipase A2; and
(c) avidin operably linked to both the compound of Formula V and the biotinylated phospholipase A2;
wherein the nucleic acid delivery peptide complex reversibly binds to nucleic acid.

The present invention provides a nucleic acid delivery peptide complex, comprising

(SEQ ID NO: 10)
(Cys-Y-Lys_{acr}-Z-LYs_{acr}-Y-CYs)ₙ

operably linked to

-L¹-R¹

wherein Lys_{acr} is a lysine modified with acridine at the s-amine; Cys is Cysteine; Lys is Lysine; Y is Lys or a covalent bond; Z is Lysine, Arginine, Leucine, Phenylalanine, or Cysteine, wherein Z is optionally conjugated by a maleimide linkage to poly(ethylene glycol); and n is 1 to 30; and wherein L¹ is a linker and is bound to Cys at the amino-terminus, the carboxy-terminus or an amino acid side-chain; wherein; wherein R¹ is one or more of a peptide, polyethylene glycol (PEG), a carbohydrate, and/or is a hydrogen; and wherein the nucleic acid delivery peptide reversibly binds to nucleic acid.

### BRIEF DESCRIPTION OF THE FIGURES

FIGS. 1A-1D. RP-HPLC Analysis of Polyacridine-Melittin Peptide Synthesis. The reaction monitoring of poly(Acr-Arg)₄-Cys (SEQ ID NO: 11) with thiolpyr-Cys-melittin at time 0 at 1.7:1 mol ratio is illustrated in panel A. The RP-HPLC chromatograms were produced by injecting 2 nmol then eluting with 0.1% TFA (v/v) and an acetonitrile gradient of 10-55% over thirty minutes while monitoring absorbance at 280 nm. The reaction monitoring at 12 hrs established the complete consumption of thiolpyr-Cys-melittin with formation of the product poly(Acr-Arg)₄-SS-melittin (SEQ ID NO: 78) and by-products of dimeric poly(Acr-Arg)₄-Cys₂ (SEQ ID NO: 12) and thiolphene (panel B). LC-ESI-MS of purified poly(Acr-Arg)₄-SS-melittin (SEQ ID NO: 78) produced ions of 1762.7 m/z and 1322.3 m/z, respectively (inset), corresponding to a mass of 5285.2 amu (panel C). Reduction of poly(Acr-Arg)₄-SS-melittin (SEQ ID NO: 78) with TCEP resulted in formation of equi-mol amounts of poly(Acr-Arg)₄-Cys (SEQ ID NO: 11) and Cys-melittin (SEQ ID NO: 52) (panel D).

FIGS. 2A-2C. Relative Binding Affinity of Polyacridine-Melittin Peptides to DNA. The concentration dependent displacement of the thiazole orange from DNA by polyacridine-melittin peptides was used to establish relative affinity. Cys-melittin (SEQ ID NO: 52) bound weakly to DNA resulting in an asymptote in the fluorescence intensity at approximately 0.5 nmols of peptide per μg of DNA, compared to 0.35 for (Acr-Arg)₄-Cys (SEQ ID NO: 11) and 0.25 for poly(Acr-Arg)₄-SS-melittin (SEQ ID NO: 78) (panel A). Comparison of poly(Acr-Arg)₂₋₄-SS-melittin (SEQ ID NOS 76-78, respectively) established poly(Acr-Arg)₂-SS-melittin (SEQ ID NO: 76) was the weakest binding resulting in an asymptote at 0.35 nmols of peptide per μg of DNA whereas poly(Acr-Arg)_{3 and 4}-SS-melittin (SEQ ID NOS 77 & 78, respectively) were nearly equivalent in displacing thiazole orange (panel B). An equal DNA binding affinity was demonstrated for poly(Acr-Arg)₄-SS-melittin (SEQ ID NO: 78), poly(Acr-Leu)₄-SS-melittin (SEQ ID NO: 81) and poly(Acr-Lys)₄-SS-melittin (SEQ ID NO: 80), each resulting in polyplex formation at 0.25 nmols of peptide per μg of DNA (panel C). FIG. 2A discloses “Poly(Acr-Arg)₄-Mal-melittin” as SEQ ID NOS 11 & 67, respectively.

FIGS. 3A-3E. Atomic Force Microscopy of Polyacridine-melittin Polyplexes. The size and shape of pGL3 (A) or polyacridine-melittin DNA polyplexes (B-E) were determined by atomic force microscope. Poly(Acr-Arg)₂-SS-melittin (SEQ ID NO: 76) (B), poly(Acr-Leu)₄-SS-melittin (SEQ ID NO: 81) (C), poly(Acr-Arg)₄-SS-melittin (SEQ ID NO: 79) (D), and poly(Acr-Lys)₄-Mal-melittin (SEQ ID NOS 71 & 67, respectively) (E) pGL3 polyplexes are compared. The results establish that the weaker DNA binding of poly(Acr-Arg)₂-SS-melittin (SEQ ID NO: 76) results in aggregated polyplexes.

FIGS. 4A-4C. Membrane Lytic Potency of Polyacridine-melittin Peptides. The hemolytic activity of melittin and polyacridine-melittin peptides were compared at pH 7.4 and 5 using a RBC hemolysis assay. The results establish Cys-melittin (SEQ ID NO: 52) is equally potent as natural melittin at pH 7.4 (panel A), but more potent at pH 5 (panel B). In comparison, poly(Acr-Arg)₄-SS-melittin (SEQ ID NO: 78) retains nearly full hemolytic potency at pH 7.4 (panel A) but is less potent at pH 5. However, when combined with pGL3, the pH 7.4 hemolytic activity of both Cys-melittin (SEQ ID NO: 52) and melittin is nearly unchanged, whereas poly(Acr-Arg)₄-SS-melittin (SEQ ID NO: 78) is non hemolytic (panel C). These results establish the masking of poly(Acr-Arg)₄-SS-melittin (SEQ ID NO: 78) membrane lytic activity while bound to DNA. FIG. 4C discloses “Poly(Acr-Arg)₄-Mal-melittin” as SEQ ID NOS 11 & 67, respectively.

FIG. 5. Comparative In Vitro Gene Transfer Potency of Polyacridine-melittin Polyplexes. CHO cells were transfected with increasing amounts of peptide (0.1, 0.35, 0.5, and 0.75 nmols peptide per μg DNA) combined with 10 μg pGL3. The luciferase expression determined at 24 hrs represents the mean and standard deviation for three independent transfections. FIG. 5 discloses SEQ ID NOS 13, 76-78, and 11 & 67, respectively, in order of appearance.

FIGS. 6A-6C. Cell Dependent In Vitro Gene Transfer Potency of Polyacridine-melittin Polyplexes. The relative gene transfer efficiency of DNA polyplexes was determined in CHO (A), 3T3 (B), and HepG2 (C) cells. Transfections were performed with 10 μg of pGL3 polyplex prepared with 0.5 nmols of peptide per μg of DNA. The gene transfer efficiency mediated by polyacridine-melittin peptides was compared to PEI (N/P 9:1) DNA polyplexes and WK₁₈ DNA polyplexes (SEQ ID NO: 13). The bars indicate transfection pGL3 combined with 1) PEI, 2) WK₁₈ (SEQ ID NO: 13), 3) Dimeric-melittin (Core sequence disclosed as SEQ ID NO: 74), 4) Cys-melittin (SEQ ID NO: 52), 5) poly(Lys(Ac)-Arg)₄-SS-melittin (SEQ ID NO: 79), 6) poly(Acr-Arg)₂-SS-melittin (SEQ ID NO: 76), 7) poly(Acr-Arg)₃-SS-melittin (SEQ ID NO: 77), 8) poly(Acr-Arg)₄-SS-melittin (SEQ ID NO: 78), 9) poly(Acr-Arg)₄-Mal-melittin (SEQ ID NOS 11 & 67, respectively), 10) poly(Acr-Lys)₄-SS-melittin (SEQ ID NO: 80), 11) poly(Acr-Lys)₄-Mal-melittin (SEQ ID NOS 71 & 67, respectively), 12) poly(Acr-Leu)₄-SS-melittin (SEQ ID NO: 81), and 13) poly(Acr-Leu)₄-Mal-melittin (SEQ ID NOS 72 & 67, respectively). The luciferase expression was determined at 24 hrs. The results represent the mean and standard deviation for three independent transfections.

FIGS. 7A-7B. Preparation of PEGylated Polyacridine Bioconjugates. PEG_{5000 Da}-maleimide was reacted with the Cys residue on Cys-Trp-Lys₃ (SEQ ID NO: 14), Cys-Trp-Lys₄ (SEQ ID NO: 15), and Cys-Trp-Lys₃ (SEQ ID NO: 16) to form PEG-Cys-Trp-(Lys)_{3, 4 and 5} (SEQ ID NOS 17-19, respectively). Activated 6-(9-acridinylamino) hexanoic acid was reacted with ε-amines of Lys to form PEG-Cys-Trp-(Lys-(Acr))_{3, 4 and 5} (SEQ ID NOS 3-5, respectively). Alternatively, mono and bis PEG-amine were reacted with activated 6-(9-acridinylamino) hexanoic acid to prepare mono-Acr-PEG and bis-Acr-PEG.

FIGS. 8A-8C. RP-HPLC Analysis of PEG-Cys-Trp-(Lys-(Acr))₅ (SEQ ID NO: 5). Cys-Trp-(Lys)₅ (SEQ ID NO: 16) elutes at 10 min on RP-HPLC eluted with 0.1% TFA and an acetonitrile gradient 5-60% over 30 min while detecting Trp by Abs at 280 nm (panel A). The inset shows the ESI-MS of Cys-Trp-Lys₅ (SEQ ID NO: 16) with an [M+H]=982.8 Da. Purified PEG-Cys-Trp-(Lys)₅ (SEQ ID NO: 19) elutes at 24 min under identical gradient and detection (panel B). Purified PEG-Cys-Trp-(Lys(Acr))₅ (SEQ ID NO: 5) elutes as a broad peak at 25 min and is detected by Abs 409 nm due to acridine (panel C).

FIGS. 9A-9C. Mass Spectral Analysis of PEG-Cys-Trp-(Lys(Acr))₅ (SEQ ID NO: 5) MALDI-TOF MS analysis PEG_{5000 Da}-maleimide resulted in an average mass with m/z of 5425 (panel A). MS analysis of PEG-Cys-Trp-(Lys)₅ (SEQ ID NO: 19) resulted in a mass shift of 1121 Da, consistent with the formation of the PEG-peptide with m/z of 6546 (panel B). Following conjugation of five 6-(9-acridinylamino)hexanoic acid residues (290 g/mol), the average mass increases by 1307 Da, consistent with the formation of PEG-Cys-Trp-(Lys(Acr))₅ (SEQ ID NO: 5) with m/z of 7853 (panel C).

FIGS. 10A-10B. Relative Binding Affinity of Mono-Acridine-PEG, Bis-Acridine-PEG, and Polyacridine Peptides with DNA. The relative binding affinity of mono and bis-acridine-PEG, and PEG-Cys-Trp-(Lys(Acr))_{3, 4, and 5} (SEQ ID NOS 3-5, respectively) for DNA was determined using a thiazole orange dye displacement assay and agarose gel band shift assay. Thiazole orange displacement established weak DNA binding for mono () and bis-acridine-PEG (▾), with higher and indistinguishable affinity determined for PEG-Cys-Trp-(Lys(Acr))_{3(▪), 4(▴), and 5(♦)} (SEQ ID NOS 3-5, respectively) (panel A). The DNA binding affinity was also compared by agarose gel electrophoresis DNA migration (1 μg) (lane 1) relative to polyplexes prepared with 1 nmol each of mono-acridine PEG (lane 2), bis-acridine PEG (lane 3), PEG-Cys-(Lys(Acr))₃ (SEQ ID NO: 20) (lane 4), PEG-Cys-(Lys(Acr))₄ (SEQ ID NO: 21) (lane 5), or PEG-Cys-(Lys(Acr))₅ (SEQ ID NO: 22) (lane 6) (Panel B). The results established maximal binding affinity with PEG-Cys-(Lys(Acr))₅ (SEQ ID NO: 22) (lane 6).

FIGS. 11A-11C. Particle Size and Zeta Potential of Polyacridine-Peptide DNA Polyplexes. The mean diameter size and zeta potential of DNA polyplexes prepared with 0.1, 0.2, 0.4, 0.8, 1.2, 1.6, or 2 nmol of PEG-Cys-(Lys(Acr))_{3, 4, and 5} (SEQ ID NOS 20-22, respectively) per μg of DNA were compared. At 0.2 nmols of polyacridine peptide or higher, PEG-Cys-(Lys(Acr))₃ (SEQ ID NO: 20) formed polyplexes of apparent mean diameter of 200 nm that remained unchanged throughout the titration (Panel A, dashed line). In contrast, the zeta potential progressively increased from −15 mV to approximately −2 mV when titrating from 0.1-2 nmol per μg of DNA (panel A, solid line). Nearly identical results were obtained when titrating with PEG-Cys-(Lys(Acr))₄ (SEQ ID NO: 21) (panel B) and PEG-Cys-(Lys(Acr))₅ (panel C) (SEQ ID NO: 22).

FIGS. 12A-12D. Atomic Force Microscopy Analysis of Plasmid DNA and Polyacridine DNA Polyplexes. AFM was used to compare the relative morphology of plasmid DNA or PEG-Cys-Trp-(Lys(Acr))₅ (SEQ ID NO: 5) DNA polyplexes bound to electropositively charged mica in panels A-C. Plasmid DNA appears as an open circular structure (panel A) of comparable dimensions relative to PEG-Cys-Trp-(Lys-Acr)₅ (SEQ ID NO: 5) DNA polyplexes prepared at either 0.2 nmol per μg of DNA (panel B) or 1 nmol per μg of DNA (panel C). Alternatively, a cationic polyplex prepared with polyacridine melittin binds to electronegative mica and appears as a collapsed structure (panel D). The results establish polyacridine PEG peptides bind to DNA to form electronegative open polyplexes that possess similar morphology as plasmid DNA.

FIGS. 13A-13H. Relative Metabolic Stability of Mono-Acridine-PEG, Bis-Acridine-PEG, and Polyacridine Peptides DNA Polyplexes. The relative metabolic stability of DNA polyplexes was compared with plasmid DNA by agarose gel electrophoresis. Plasmid DNA (1 μg) (panel A), or DNA polyplexes prepared with 1 nmol each of mono-acridine PEG (panel B), bis-acridine PEG (panel C), PEG-Cys-(Lys(Acr))₃ (SEQ ID NO: 20) (panel D), PEG-Cys-(Lys(Acr))₄ (SEQ ID NO: 21) (panel E), or PEG-Cys-(Lys-(Acr))₅ (SEQ ID NO:22) (panel) were incubated with 0.06 U of DNase at 37° C. for 0 (lane 1), 5 (lane 2), 10 (lane 3) and 20 (lane 4) min. PEG-Cys-(Lys(Acr))₅ (SEQ ID NO: 22) DNA polyplexes were also prepared at 0.2 (panel G) and 0.4 (panel H) nmol of polyacridine peptide per μg of DNA and digested with DNase. The results demonstrate that PEG-Cys-(Lys-(Acr))₅(SEQ ID NO: 22) provided the greatest protection at 1 nmol per μg of DNA, (panel F) while the lower stoichiometries of 0.2 or 0.4 nmol per μg of DNA (panel G and H) resulted in less stability.

FIG. 14. In Vivo Gene Expression Mediated by Polyacridine Peptide DNA Polyplexes. The gene transfer efficiencies of naked DNA () or polyacridine peptide DNA polyplexes prepared with 0.2 (▴), 2 (◯), or 4 (Δ) nmol of PEG-Cys-(Lys-(Acr))₅ (SEQ ID NO: 22) per μg of DNA were determined following i.m. dosing and electroporation of 1 μg of pGL3 in the gastrocnemius muscle of ICR male mice (n=4). The luciferase expression was quantified at times ranging from 2-14 days by bioluminescence imaging (BLI) following an i.m. dose of luciferin. Open polyplexes prepared at 0.2 nmol (▴) of PEG-Cys-(Lys-(Acr))₅ (SEQ ID NO: 22) per μg of DNA showed the lowest transfection efficiency, while polyplexes prepared at 2 nmol (◯) of PEG-Cys-(Lys-(Acr))₅ (SEQ ID NO: 22) per μg of DNA showed similar expression levels to that of naked DNA. Increasing the stoichiometry to 4 nmol (Δ) PEG-Cys-(Lys-(Acr))₅ (SEQ ID NO: 22) per μg of DNA resulted in a more sustained expression. The results represent the mean and standard deviation of four doses.

FIG. 15. Synthesis of a Man 9 Glycopeptide. A high-mannose N-glycan containing a single Asn residue was purified from soybean agglutinin and then functionalized to possess an N-terminal Tyr and propionate maleimide. The resulting Man 9 Mal was reacted with Cys-(Acr-Lys)₄ (SEQ ID NO: 23), which was prepared by solid phase peptide synthesis using Fmoc-Lys(Acr). The resulting Man 9 glycopeptide possesses affinity for DC-SIGN and for binding to plasmid DNA through polyintercalation.

FIGS. 16A-16D. RP-HPLC Analysis of Man 9 Derivatives. Man 9 Asn obtained from SBA was N-terminally modified with Boc-Tyr then analyzed for purity by RP-HPLC (panel A). The removal of Boc from Tyr with TFA results in an earlier retention time (panel B). Conjugation of an N-terminal propionate maleimide increased the retention time (panel C) and conjugation of Man 9 Mal with Cys-(Acr-Lys)₄ (SEQ ID NO: 23) resulted in the formation of Man 9 glycopeptide that rechromatographed as a single peak on RP-HPLC (panel D). The inset in panel D illustrates the ESI-MS analysis of the purified glycopeptide. See FIG. 1 for structures.

FIG. 17. Gycopeptide DNA Binding By Agarose Gel Band Shift. A DNA band shift assay was performed by adding increasing amounts of glycopeptide to plasmid DNA. Lane 1 represents control plasmid DNA (1 μg) whereas lanes 2-5 are the result of adding 0.2, 0.4, 0.6 and 1 nmol of Man 9 glycopeptide to 1 μg of DNA.

FIG. 18. Glycopeptide DNA Binding by Fluorophore Displacement Assay. The displacement of a thiazole orange intercalator dye by increasing amounts of Man 9 glycopeptide or Cys-(Acr-Lys)₄ (SEQ ID NO: 23) results in an asymptote in the fluorescence intensity at 0.2-0.4 nmol per μg of DNA.

FIG. 19. Cy5-DNA Glycopeptide Polyplex Binding to DC-SIGN Expressing CHO (+) Cells. The results of FACS analysis of Cy5-DNA Man 9 glycopeptide polyplex mediated uptake in DC-SIGN CHO (+) and (−) cells are illustrated. The fluorescence intensity in CHO (+) was nearly 10-fold higher than CHO (−) cells whereas, untreated CHO (+) or (−) cells demonstrated negligible fluorescence intensity.

FIG. 20. In Vitro Transfection of CHO (+) and (−) DC-SIGN Cells. The result of in vitro transfection of CHO (+) (open bar) and (−) (closed bar) with different gene transfer agents is illustrated. Transfection of CHO (+) and (−) cells with PEI DNA polyplexes (N:P of 9) leads to high level of luciferase expression which is non selective. Likewise, transfection with Cys-(Acr-Lys)₄ (SEQ ID NO: 23) peptide polyplexes results in lower levels of non-selective gene transfer. Alternatively, transfection with Man 9 glycopeptide (GP) polyplexes results in a 100-fold selective gene transfer in CHO (+) cells over CHO (−) cells. Each result is the mean and standard deviation of six independent transfections.

FIG. 21. Synthetic Strategy for PEGylated Polyacridine Peptides. The approach used to prepare PEGylated-Mal-(Acr-Arg)₄ (SEQ ID NO: 24) and PEGylated-SS-(Acr-Arg)₄ (SEQ ID NO: 25) is demonstrated as an example of how all other polyacridine PEG-peptides described in Table 1 were prepared. (Acr-Arg)₄-Cys (SEQ ID NO: 11) (where Acr is Lys modified on the s-amine with an acridine) was prepared by solid phase peptide synthesis. The Cys thiol was then reacted with either 5 kDa PEG-maleimide or PEG-OPSS (n=109), resulting in PEGylated-Mal-(Acr-Arg)₄ (SEQ ID NO: 24) or PEGylated-SS-(Acr-Arg)₄ (SEQ ID NO: 25).

FIGS. 22A-22C. RP-HPLC Analysis of Polyacridine PEG-Peptide Synthesis. Reaction of (Acr-Arg)₄-Cys (SEQ ID NO: 11) with 1.1 mol equivalents of PEG-Mal (panel A), results in the formation of PEGylated-Mal-(Acr-Arg)₄ (SEQ ID NO: 24) detected at 280 nm with simultaneous consumption of (Acr-Arg)₄ (SEQ ID NO: 26) and formation of dimeric peptide ((Acr-Arg)₄-Cys)₂ (Core sequence disclosed as SEQ ID NO: 27) (panel B). The HPLC purified product PEGylated-Mal-(Acr-Arg)₄ (SEQ ID NO: 24) rechromatographed on RP-HPLC as a single peak (panel C) and is characterized by MALDI-TOF MS (panel C, inset), resulting in an observed m/z corresponding to the calculated mass (Table 1). The preparation of PEGylated-Mal-(Acr-X)ₙ and PEGylated-SS-(Acr-X)ₙ peptides described in Table 1 produced equivalent chromatographic evidence. FIGS. 22A-22C discloses SEQ ID NOS 11, 27, 24, and 24, respectively, in order of appearance.

FIGS. 23A-23B. DNA Binding Affinity of PEGylated Polyacridine Peptides. A thiazole orange displacement assay was used to determine the relative binding affinity of polyacridine PEG peptides for DNA. Polyacridine PEG peptides when titrating 0.2 to 1 nmol of PEGylated (Acr-Arg)₄ (SEQ ID NO: 28) (), PEGylated (Acr-Lys)₄ (SEQ ID NO: 29) (◯), PEGylated (Acr-Leu)₄ (SEQ ID NO: 30) (▾), or PEGylated (Acr-Glu)₄ (SEQ ID NO: 31) (Δ), with 1 μg of pGL3 and 0.1 μM thiazole orange in 0.5 ml of 5 mM Hepes pH 7.0 prior to measuring thiazole fluorescence intensity. The results in Panel A established that PEGylated (Acr-Arg)₄ (SEQ ID NO: 28) and PEGylated (Acr-Lys)₄ (SEQ ID NO: 29) possessed higher affinity for DNA compared to PEGylated (Acr-Glu)₄ (SEQ ID NO: 31) and PEGylated (Acr-Leu)₄ (SEQ ID NO: 30). In Panel B the relative affinity of PEGylated (Acr-Lys)₂ (SEQ ID NO: 32), PEGylated (Acr-Lys)₄ (SEQ ID NO: 29), and PEGylated (Acr-Lys)₆ (SEQ ID NO: 33) are compared. The results establish that both the number of Acr and the spacing amino acid contribute to the DNA binding affinity.

FIGS. 24A-24F. Size and Charge of PEGylated Polyacridine Polyplexes. The QELS particle size (--▴--) and zeta potential (--) of polyplexes, prepared at concentrations ranging from 0.2-1 nmol of peptide per μg of DNA, are illustrated for PEGylated (Acr-Arg)₄ (SEQ ID NO: 28) (A), PEGylated (Acr-Leu)₄ (SEQ ID NO: 30) (B), PEGylated (Acr-Glu)₄ (SEQ ID NO: 31) (C), PEGylated (Acr-Lys)₂ (SEQ ID NO: 32) (D), PEGylated (Acr-Lys)₄ (SEQ ID NO: 29) (E) or PEGylated (Acr-Lys)₆ (SEQ ID NO: 33) (F). The results establish no significant change in particle size throughout the titration, whereas the zeta potential increases from −20 to 0 mV when titrating with peptides containing spacing amino acids Arg, Lys or Leu (panels A, B, D). Comparison of PEGylated (Arc-Lys)ₙ repeats of n=2, 4 and 6 (SEQ ID NOS 32, 29, and 33, respectively) (panel D, E and F) results in polyplexes that titrate to final zeta potential of −10, −2 and 5 mV, respectively.

FIGS. 25A-25H. Shape of PEGylated Polyacridine Polyplexes. Atomic force microscopy (AFM) was used to analyze the shape of DNA polyplexes prepared at 0.8 nmol per μg of DNA with (A) PEGylated (Acr-Arg)₄ (SEQ ID NO: 28) (+) mica, (B) PEGylated (Acr-Lys)₄ (SEQ ID NO: 29) (+) mica, (C) PEGylated (Acr-Leu)₄ (SEQ ID NO: 30) (+) mica, (D) PEGylated (Acr-Glu)₄ (SEQ ID NO: 31) (+) mica, or (E) pGL3 (+) mica, (F) 0.2 nmol of PEGylated (Acr-Lys)₆ (SEQ ID NO: 33) (+) mica, (G) 0.8 nmol of PEGylated (Acr-Lys)₆ (SEQ ID NO: 33) (−) mica, and (H) 0.8 nmol of PEGylated (Acr-Lys)₆ (SEQ ID NO: 33) (+) mica. Anionic PEGylated polyacridine polyplexes produced open polyplex structures (A-D, F) that appeared slightly more coiled than plasmid DNA (E), where cationic PEGylated polyacridine polyplexes produced closed polyplex structures (G). Panel H demonstrates that cationic polyplexes do not bind to cationic mica. Each inset represents a 1×1 μm enlargement.

FIGS. 26A-26C. Metabolic Stability of PEGylated Polyacridine Polyplexes. Agarose gel electrophoresis of (1) plasmid DNA, (2) PEGylated (Acr-Lys)ₙ polyplex (n=2, 4 or 6) (SEQ ID NOS 32, 29, and 33, respectively) at 0.2 nmol of peptide per μg of DNA, (3) PEGylated (Acr-Lys)ₙ polyplex at 0.8 nmol of peptide per μg of DNA, (4) release of DNA from PEGylated (Acr-Lys)ₙ polyplex at 0.8 nmol per μg of DNA, (5) PEGylated (Acr-Lys)ₙ polyplex at 0.2 nmol per μg of DNA following DNase digest, (6) released PEGylated (Acr-Lys)ₙ polyplex at 0.2 nmol per μg of DNA following DNase digest, (7) PEGylated (Acr-Lys)ₙ polyplex at 0.8 nmol per μg of DNA following DNase digest, (8) released PEGylated (Acr-Lys)ₙ polyplex at 0.8 nmol per μg of DNA following DNase digest. The results establish the partial or complete protection of DNA from DNase at 0.8 nmol of (Acr-Lys)₂-PEG (SEQ ID NO: 32) (panel A lane 8) and (Acr-Lys)₄-PEG (SEQ ID NO: 29) (panel B lane 8), and the complete protection of DNA from DNase at 0.2 and 0.8 nmol of (Acr-Lys)₆-PEG (SEQ ID NO: 33) (panel C lane 6 and 8).

FIGS. 27A-27B. Stimulated In Vivo Gene Expression Using PEGylated Polyacridine Polyplexes. Direct HD dosing of 1 μg of pGL3 in multiple mice results in a mean BLI response of 10⁸ photons/sec/cm²/sr at 24 hrs following dosing (panel A, HD DNA). Alternatively, a 24 hr BLI analysis of mice tail vein dosed with pGL3 (1 μg in 50 μl) in complex with 0.5 nmol of either PEGylated-Mal-(Acr-Arg)₄ (SEQ ID NO: 24) (panel A, Mal) or PEGylated-SS-(Acr-Arg)₄ (SEQ ID NO: 25) (panel A, SS) followed by hydrodynamic stimulation with 2 ml of saline delivered 30 min after DNA delivery, results in approximately 10⁷ photons/sec/cm²/sr (panel A). Omission of HD stimulation (not shown) or PEGylated polyacridine peptide (panel A, i.v. DNA) results in no expression (10⁵ photons/sec/cm²/sr). Likewise, HD stimulation after 30 min failed to produce measurable expression from a 1 μg pGL3 dose in complex with PEG-Cys-Trp-Lys₁₈ (SEQ ID NO: 34) (panel A, Cont 1) or a PEGylated glycoprotein described previously⁴¹ (panel A, Cont 2). An acetic acid counter ion on PEGylated-Mal-(Acr-Arg)₄ (SEQ ID NO: 24) resulted in nearly 10-fold increase in expression relative to a TFA counter ion (panel A). Varying the dwell time of the HD stimulation from 5-120 min following a 1 μg dose of pGL3 in complex with 0.5 nmol of PEGylated-Mal-(Acr-Arg)₄ (SEQ ID NO: 24) established a maximum of 30 min to retain expression at 10⁷ photons/sec/cm²/sr or higher measured at 24 hrs post administration (panel B). Statistical analysis was performed using a two-tailed unpaired t-test (*p≦0.05).

FIGS. 28A-28C. Structure-Activity Relationships for Stimulated Gene Expression. The BLI analysis at 24 hrs following tail vein dosed and HD stimulated (30 min post-DNA administration) pGL3 (1 μg in 50 μl) in complex with 0.5 nmol of either (Acr-Arg)₄-Cys-Mal-PEG (SEQ ID NO: 35) (panel A, Arg), 0.6 nmol of (Acr-Lys)₄-Cys-Mal-PEG (SEQ ID NO: 36) (panel A, Lys), 1 nmol of (Acr-Leu)₄-Cys-Mal-PEG (SEQ ID NO: 37) (panel A, Leu), or 0.8 nmol of (Acr-Glu)₄-Cys-Mal-PEG (SEQ ID NO: 38) (panel A, Glu) are compared with direct HD delivery of 1 μg of pGL3. The results establish polyacridine PEG-peptides with Arg and Lys spacing amino acids mediate 10⁷-10⁸ photons/sec/cm²/sr whereas substitution with Leu and Glu results in negligible expression. Varying only the stoichiometry of PEGylated polyacridine peptide to DNA for (Acr-Arg)₄-Cys-Mal-PEG (SEQ ID NO: 35) (panel B, Arg) and (Acr-Lys)₄-Cys-Mal-PEG (SEQ ID NO: 36) (panel B, Lys), established a maximal expression at 0.6 for Arg and 0.8 for Lys (panel B). Direct comparison of HD stimulated gene expression using (Acr-Lys)ₙCys-Mal-PEG (where n=2, 4, or 6) (SEQ ID NOS 40, 36, and 39, respectively) in complex with 1 m of pGL3 established the equivalency of 0.8 of (Acr-Lys)₄-Cys-Mal-PEG (SEQ ID NO: 36) with 0.2 nmol of (Acr-Lys)₆-Cys-Mal-PEG (SEQ ID NO: 39), respectively (panel C), relative to (Acr-Lys)₂-Cys-Mal-PEG (SEQ ID NO: 40) which mediated negligible expression. Statistical analysis was performed using a two-tailed unpaired t-test (*p≦0.05). FIG. 28B discloses SEQ ID NOS 28 and 29, respectively and FIG. 28C discloses SEQ ID NOS 32, 29, and 33, respectively, in order of appearance.

FIGS. 29A-29C. Optimal Parameters for Stimulated Gene Expression of Polyacridine PEG-peptide DNA Polyplexes. In panel A, the level of expression measured at 24 hrs, following HD stimulation 30 min after DNA dosing, remains nearly constant when delivering (Acr-Lys)₆-Cys-Mal-PEG (SEQ ID NO: 39) pGL3 polyplexes prepared at stoichiometries ranging from 0.2-0.8 nmols of peptide per μg of DNA (panel A). The results in panel B illustrate that varying the HD stimulation delay-time following delivery of (Acr-Lys)₆-Cys-Mal-PEG (SEQ ID NO: 39) pGL3 polyplexes results in expression of approximately 10⁸ photons/Sec/cm²/sr up to 60 min, whereas the expression decreased nearly 100-fold when delaying HD stimulation to 120 min (panel B). The dose-response curve for in vivo gene expression mediated delivery of (Acr-Lys)₆-Cys-Mal-PEG (SEQ ID NO: 39) pGL3 polyplexes with 5 min delay in stimulation () is compared with direct HD of pGL3 (◯). The luciferase expression at 24 hrs determined by BLI established that HD delivery of 1 μg of (Acr-Lys)₆-Cys-Mal-PEG polyplex (SEQ ID NO: 39) is approximately 5-fold more efficient than the HD delivery of pGL3 (panel C). FIGS. 29A and 29C disclose SEQ ID NO 33.

FIGS. 30A-30F. Pharmacokinetic and Biodistribution Analysis of PEGylated Polyacridine Polyplexes. The pharmacokinetic profile for PEGylated (Acr-Lys)₂ (SEQ ID NO: 32), PEGylated (Acr-Lys)₄ (SEQ ID NO: 29), and PEGylated (Acr-Lys)₆ ¹²⁵I-DNA polyplexes (SEQ ID NO: 33) is compared with 1251-DNA (panel A). Extraction of the ¹²⁵I-DNA from blood time points followed by agarose electrophoresis and autoradiography produced the images in C—F. Biodistribution analysis of PEGylated polyplexes resulted in comparison of the liver accumulation and elimination over time (panel B). The results establish that PEGylated (Acr-Lys)₆ (SEQ ID NO: 33) stabilizes DNA in the blood for up to two hours. FIG. 30B discloses SEQ ID NOS 32, 29, and 33, respectively, in order of appearance. FIGS. 30D, 30E, and 30F disclose SEQ ID NOS 32, 29, and 33, respectively.

FIGS. 31A and 31B. FIG. 31A shows (Acr-Lys)₆-Cys-Biotin (SEQ ID NO: 41). FIG. 31B is a schematic of the PLA2-DNA complex. FIG. 31B discloses “(AK)₆-Biotin” as SEQ ID NO: 83.

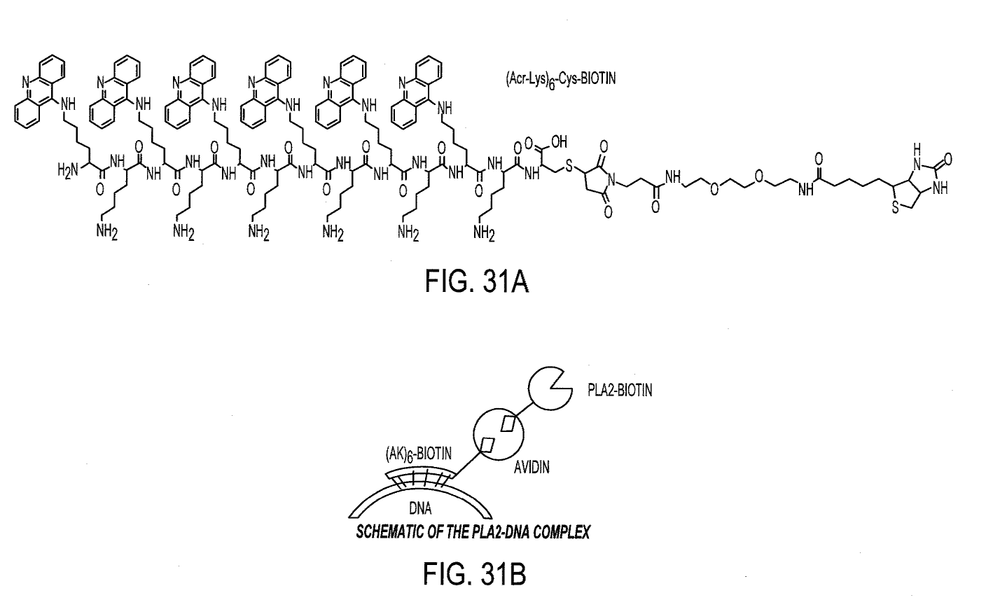

FIG. 32. IM-EP of Man9-(Acr-Lys)₆ (SEQ ID NO: 42) DNA in Mice. The results demonstrate the influence of glycopeptide to DNA ratio on the level of gene expression over time.

FIG. 33. Formulation of CKK_{acr}C_{mal-mPEG}K_{acr}KC (SEQ ID NO: 43), Formula VII.

### DETAILED DESCRIPTION

The nucleic acid delivery peptide complex of the present invention comprising several moieties: a peptide backbone to which acridines can be bonded, possibly one or more functional moieties, and a linker that operably links the peptide backbone to the functional moiety/moieties. In certain embodiments, a nucleic acid molecule is reversibly bound to one or more of the peptide complexes described above. The present invention further provides an anionic open polyplex comprising a nucleic acid molecule reversibly bound to a nucleic acid delivery peptide complex.

Acridines

Acridine, C₁₃H₉N, is an organic compound that is a colorless solid that is commonly used for dyes and drugs. Acridine and related derivatives bind to DNA and RNA due to their abilities to intercalate. As used herein the term “acridine” is used to mean substituted or unsubstituted acridine and their related derivatives. Examples of substituted acridines are known in the literature, such as in Table 1 of Teh Kuei Che, Rosario Fico and E. S. Canellakis “Diacridines, Bifunctional Intercalators. Chemistry and Antitumor Activity, J. Medicinal Chemistry, (1978) 21, 868-874 and in Table 2 of Marc O. Anderson, John Sherrill, Peter B. Madrid, Ally P. Liou, Jennifer L. Weisman, Joseph L. DeRisi, and R. Kiplin Guy “Parallel Synthesis of 9-Aminoacridines and their evaluation against Cloroquine-resistant Plasmodium Falciparum” Bioorganic and Medicinal Chemistry, (2006) 14, 334-343.

Peptide Backbones

The nucleic acid delivery peptide complexes of the present invention contain a peptide backbone. In certain embodiments, the backbone is (A¹-A²)ₙ, where A¹ and A² are amino acids, and n is 1-10, or the backbone is (Lys)ₙ (SEQ ID NO: 44), where n is 1-6. In certain embodiments, A¹ is Lysine, Arginine, Leucine or Glutamic acid and A² is 1 to 6 amino acids. In certain embodiments, a cysteine residue is bonded to(A¹-A²)ₙ at the amino-terminus, the carboxy-terminus or an amino acid side-chain of A².

The term “amino acid” includes the residues of the natural amino acids (e.g. Ala, Arg, Asn, Asp, Cys, Glu, Gln, Gly, H is, Hyl, Hyp, Ile, Leu, Lys, Met, Phe, Pro, Ser, Thr, Trp, Tyr, and Val) in Dextrorotary or Levorotary stereoisomeric forms, as well as unnatural amino acids (e.g., phosphoserine, phosphothreonine, phosphotyrosine, hydroxyproline, and gamma-carboxyglutamate; hippuric acid, octahydroindole-2-carboxylic acid, statine, 1,2,3,4,-tetrahydroisoquinoline-3-carboxylic acid, penicillamine, ornithine, citruline, alpha-methyl-alanine, para-benzoylphenylalanine, phenylglycine, propargylglycine, sarcosine, and tert-butylglycine). The term also comprises natural and unnatural amino acids (Dextrorotary and Levorotary stereoisomers) bearing a conventional amino protecting group (e.g. acetyl or benzyloxycarbonyl), as well as natural and unnatural amino acids protected at the carboxy terminus (e.g., as a (C₁-C₆)alkyl, phenyl or benzyl ester or amide; or as an α-methylbenzyl amide). Other suitable amino and carboxy protecting groups are known to those skilled in the art (See for example, Greene, T. W.; Wutz, P. G. M., Protecting Groups In Organic Synthesis; second edition, 1991, New York, John Wiley & sons, Inc, and documents cited therein). An amino acid can be linked to the remainder of a compound of formula (I) through the carboxy terminus, the amino terminus, or through any other convenient point of attachment, such as, for example, through the sulfur of cysteine.

The term “peptide” describes a sequence of 2 to 25 amino acids (e.g. as defined hereinabove) or peptidyl residues. The sequence may be linear or cyclic. For example, a cyclic peptide can be prepared or may result from the formation of disulfide bridges between two cysteine residues in a sequence. A peptide can be linked to the remainder of a compound of formula I through the carboxy terminus, the amino terminus, or through any other convenient point of attachment, such as, for example, through the sulfur of a cysteine. Peptide derivatives can be prepared as disclosed in U.S. Pat. Nos. 4,612,302; 4,853,371; and 4,684,620, or as described in the Examples hereinbelow. Peptide sequences specifically recited herein are written with the amino terminus on the left and the carboxy terminus on the right.

The terms “protein,” “peptide” and “polypeptide” are used interchangeably herein.

Linking Groups

In the present invention L¹ is a linking group that links the peptide backbone to a functional group. In certain embodiments L¹ is —S—, a disulfide linkage, a maleimide linkage, an amide linkage, ester linkage, ether linkage, a thiol ether linkage, a hydrazone linkage, or an acetamide linkage.

Functional Moieties

In embodiments of the present invention R¹ is linked to the peptide backbone. In certain embodiments, R¹ is a peptide different from the peptide backbone, polyethylene glycol (PEG), a carbohydrate, biotin or avidin, and/or is hydrogen.

In certain embodiments, R¹ is a carbohydrate, such as a targeting ligand. In certain embodiments, the carbohydrate is a triantennary N-glycan, such as a high-mannose N-glycan. In certain embodiments, the carbohydrate is Man₉GlcNAc₂.

In certain embodiments, R¹ is polyethylene glycol (PEG). In certain embodiments, the PEG is between 500 g/mol and 20,000 g/mol, such as 10,000 g/mol, 5,000 g/mol, 2,000 g/mol or 500 g/mol.

In certain embodiments, R¹ is a peptide, such as a nuclear localization signal (NLS) peptide, a fusogenic peptide, a targeting peptide, an NLS-fusogenic peptide. In certain embodiments, the R¹ peptide is NLS; Cys NLS; Control NLS; Cys-Control NLS; Mal-Gly-Control-NLS; Melittin (natural melittin); Cys-melittin; K-Melittin; Cys-K-Melittin; Mal-Gly-K-Melittin (maleimide); Cys-K-Melittin-NLS; Cys-NLS-K-Melittin; Modified Cys-NLS-Melittin; PC4—NLS Analogue Cys-PC4; PC4—NLS Analogue Maleimide-PC4; PC4—NLS Analogue Cys-PC4—NLS; PC4—NLS Analogue Cys-NLS-PC4; Cys-JTS or Octreotide (SEQ ID NOS 45-63, respectively).

Examples of R¹ peptides that are linked to the peptide-acridine moiety are the following:

| Nuclear Localization | | |
|---|---|---|
| Signal (NLS) Peptide | Sequence | SEQ ID NO: |
| NLS | GGPK KKRK VG | 45 |
| Cys NLS | CGPK KKRK VG | 46 |
| Control NLS | GGPKT KRKV G | 47 |
| Cys-Control NLS | CGPKT KRKV G | 48 |
| Mal-Gly-Control-NLS | Mal-GGPKT KRKV G | 49 |
| Melittin | Sequence | SEQ ID NO: |
| Melittin (natural | GIGAVLKVLTTGLPALISWIKRKRQQ | 50 |
| melittin) | | |
| Cys-melittin | CWKKGIGAVLKVLTTGLPALISLIKRKRQQ | 52 |
| (From Table 1, | | |
| Baumhover et al, | | |
| note that W and L | | |
| were swapped | | |
| and CWKK (SEQ | | |
| ID NO: 51) was | | |
| added to the N- | | |
| terminus) | | |
| K-Melittin (note | WGI GAVL KVLT TGLP ALIS LIKK KKQQ | 53 |
| that K was | | |
| substituted for R | | |
| throughout, and | | |
| W and L were | | |
| swapped) | | |
| Cys-K-Melittin | CWGI GAVL KVLT TGLP ALIS LIKK KKQQ | 54 |
| | Percent alpha helicity: 64.29% | |
| Mal-Gly-K-Melittin | Mal-GWGI GAVL KVLT TGLP ALIS LIKK KKQQ | 55 |
| (maleimide) | | |
| Cys-K-Melittin-NLS | CWGI GAVL KVLT TGLP ALIS LIKK KKQQ | 56 |
| | GPKK KRKV G | |
| | Percent alpha helicity: 48.65% | |
| Cys-NLS-K-Melittin | CGPK KKRK VGWG IGAV LKVL TTGL | 57 |
| | PALI SLIK KKKQ Q | |
| | Percent alpha helicity: 43.24% | |
| Modified Cys- | CGPK KKRK VWG IGAV LKVL TTGL PALI | 58 |
| NLS-Melittin | SLIK KKKQ Q | |
| | Percent alpha helicity: 52.78% | |
| PC4-NLS Analogues | Sequence | SEQ ID NO: |
| Cys-PC4 | CSSA WWSY WPPV A-NH₂ | 59 |
| Maleimide-PC4 | Mal-GSSA WWSY WPPV A-NH₂ | 60 |
| Cys-PC4-NLS | CSSA WWSY WPPV AGGP KKKR KVG | 61 |
| Cys-NLS-PC4 | CGGP KKKR KVGS SAWW SYWP PVA-NH₂ | 62 |
| Other compounds | Sequence | SEQ ID NO: |
| Cys-JTS | CGLFEALLELLESLWELLEA | 63 |
| Octreotide (D) D-Amino Acid X = Gly(Mal) or Iodoacetamide | 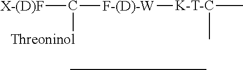 | |

In certain embodiments, the functional moiety is a protein, such as an enzyme. Examples of proteins include enzymes, such as phospholipase A2 (PLA2). In certain embodiments of the present invention, more than one functional moiety is linked to the peptide backbone. For example, a NLS and a PEG are linked to the peptide backbone. In another embodiment,

Nucleic Acid Delivery Peptide Complexes

The present invention provides a nucleic acid delivery peptide complex, comprising

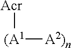

operably linked to

Cys-L¹-R¹

wherein Acr is an acridine; A¹ is Lysine, Arginine, Leucine or Glutamic acid; A² is 1 to 6 amino acids; Cys is cysteine, wherein the Cys is bound to L¹ at the amino-terminus, the carboxy-terminus or an amino acid side-chain; and n is 1 to 10; wherein L¹ is a linker; wherein R¹ is one or more of a peptide, polyethylene glycol (PEG), a carbohydrate, biotin or avidin and/or is a hydrogen; and wherein the nucleic acid delivery peptide complex reversibly binds to nucleic acid.

The present invention provides a nucleic acid delivery peptide complex having the following structure as indicated in Formula Ia, I b, Ic or Id:

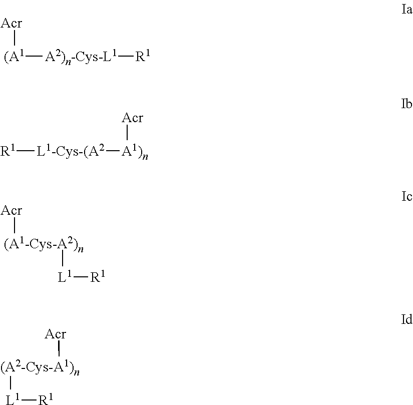

The present invention provides a nucleic acid delivery peptide complex, comprising Formula II:

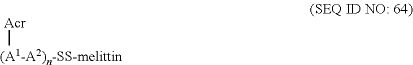

wherein
Acr is acridine;
A¹ is Lysine;
A² is Arginine, Leucine or Lysine;
n is 2 to 6;
wherein the nucleic acid delivery peptide complex binds to nucleic acid.

The present invention provides a nucleic acid delivery peptide complex, comprising Formula III:

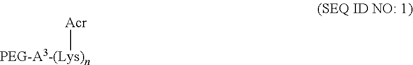

wherein
Acr is acridine;
A³ is Cys or Cys-Trp; and
n is 1 to 6;
wherein the nucleic acid delivery peptide complex binds to nucleic acid. The present invention provides a nucleic acid delivery peptide complex, comprising Formula IV:

(SEQ ID NO: 2)
Man9-L¹-(Acr-Lys)ₙ

wherein L¹ is a linker, and n is 4-6. In certain embodiments, the linker is an acetamide linkage or a maleimide linkage.

In certain embodiments, the compound is Formula IVa:

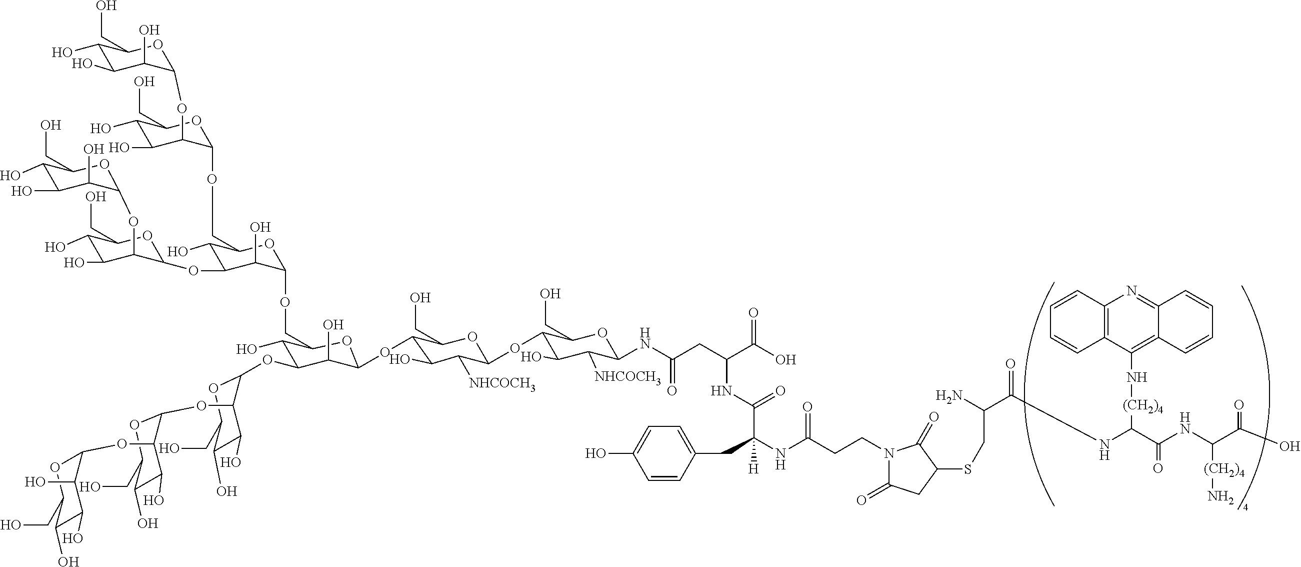

In certain embodiments the compound is Formula IVb:

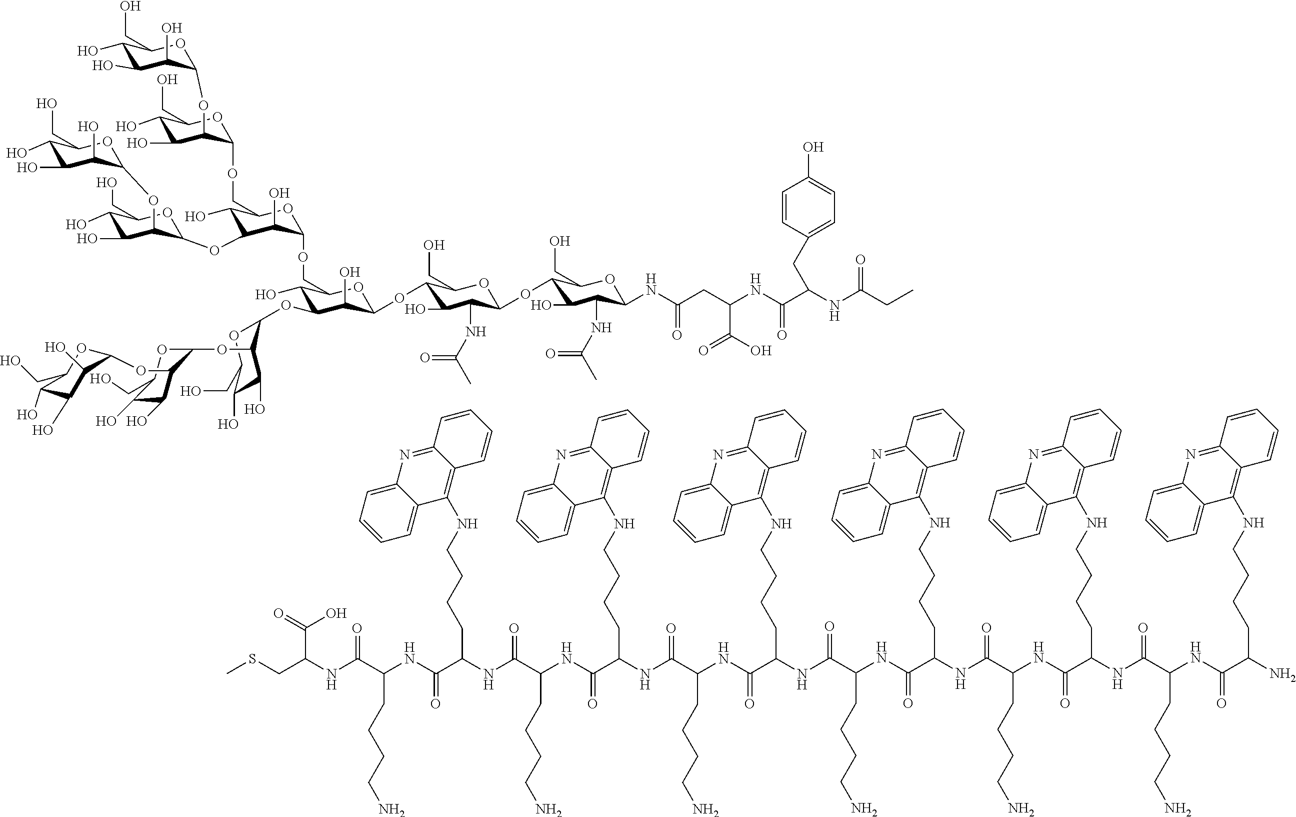

In certain embodiments, the binding of a nucleic acid delivery peptide complex to a nucleic acid molecule forms an anionic open polyplex.

In certain embodiments, the nucleic acid delivery peptide complex comprises:
(a) a compound of Formula V:

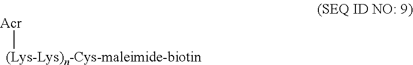

wherein n is 4-6;
(b) a biotinylated phospholipase A2; and
(c) avidin operably linked to both the compound of Formula V and the biotinylated phospholipase A2;
wherein the nucleic acid delivery peptide complex reversibly binds to nucleic acid.

Nucleic Acids of the Present Invention

In certain embodiments, the nucleic acid delivery peptide complex of the present invention is reversibly bound to a nucleic acid molecule.

The term “nucleic acid” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues. A “nucleic acid fragment” is a fraction of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA that can be single- or double-stranded, optionally containing synthetic, non-natural or altered nucleotide bases capable of incorporation into DNA or RNA polymers. The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene.

The invention encompasses isolated or substantially purified nucleic acid compositions. In the context of the present invention, an “isolated” or “purified” RNA or DNA molecule is an RNA or DNA molecule that exists apart from its native environment and is therefore not a product of nature. An isolated RNA or DNA molecule may exist in a purified form or may exist in a non-native environment such as, for example, in a transgenic host cell or bacteriophage. For example, an “isolated” or “purified” nucleic acid molecule, or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein.

The term “gene” is used broadly to refer to any segment of nucleic acid associated with a biological function. Thus, genes include coding sequences and/or the regulatory sequences required for their expression. For example, gene refers to a nucleic acid fragment that expresses mRNA, functional RNA, or specific protein, including regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

A “transgene” refers to a gene that has been introduced into the genome by transformation and is stably maintained. Transgenes may include, for example, DNA that is either heterologous or homologous to the DNA of a particular cell to be transformed. Additionally, transgenes may comprise native genes inserted into a non-native organism, or chimeric genes. The term “endogenous gene” refers to a native gene in its natural location in the genome of an organism. A “foreign” gene refers to a gene not normally found in the host organism but that is introduced by gene transfer.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have at least 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

A “functional RNA” refers to an antisense RNA, ribozyme, RNA interference molecule, or other RNA that is not translated.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell.

Compositions and Methods of Use

The present invention provides a therapeutic method for preventing or treating a pathological condition or symptom in a mammal, such as a human, by administering to the mammal a nucleic acid delivery peptide complex or an anionic open polyplex

The present invention provides a nucleic acid delivery polyplex or a nucleic acid delivery complex for use in medical therapy.

Further, the compounds of the present invention can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

The compounds of the present invention can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula Ito the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.4 μg/kg to about 100 mg/kg body weight of the recipient per day, e.g., from about 10 to about 75 μg/kg of body weight per day, such as 3 to about 50 μg/kg, such as in the range of 6 to 90 μg/kg/day, such as in the range of 15 to 60 μg/kg/day. In one embodiment, the dose is 40 μg/kg/day.

The compound is conveniently formulated in unit dosage form; for example, containing 0.1 to 1000 mg, conveniently 10 to 750 mg, most conveniently, 50 to 500 mg of active ingredient per unit dosage form. In one embodiment, the invention provides a composition comprising a compound of the invention formulated in such a unit dosage form. In certain embodiments the formulated dosage form in humans is approximately 0.1-100 mg DNA plus 0.1-100 mg of delivery peptides assuming a 90 kg patient. In certain embodiments the dose is 0.1-4 mg of DNA plus 0.1 to 4 mg of delivery peptide, assuming a 90 kg patient. One of ordinary skill in the art would be able to modify the dosage based on the body weight of the subject.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Compounds of the invention can also be administered in combination with other therapeutic agents, for example, other agents that are useful as antibiotics. Examples of such agents include a protein synthesis inhibitor, a cell wall growth inhibitor, a cell membrane synthesis inhibitor, a nucleic acid synthesis inhibitor, or a competitive enzyme inhibitor. In certain embodiments, the additional agent is an antibiotic such as penicillin, ampicillin, amoxicillin, vancomycin, cycloserine, bacitracin, cephalolsporin, imipenem, colistin, methicillin, streptomycin, kanamycin, tobramycin, gentamicin, tetracycline, chlortetracycline, doxycycline, chloramphenicol, lincomycin, clindamycin, erythromycin, oleandomycin, polymyxin nalidixic acid, rifamycin, rifampicin, gantrisin, trimethoprim, isoniazid, paraminosalicylic acid, or ethambutol.

Accordingly, in one embodiment the invention also provides a composition comprising a compound of the present invention, at least one other therapeutic agent, and a pharmaceutically acceptable diluent or carrier. The invention also provides a kit comprising a compound of the present invention, at least one other therapeutic agent, packaging material, and instructions for administering the compound of the present invention or the pharmaceutically acceptable salt thereof and the other therapeutic agent or agents to an animal.

As used herein, the term “therapeutic agent” refers to any agent or material that has a beneficial effect on the mammalian recipient. Thus, “therapeutic agent” embraces both therapeutic and prophylactic molecules having nucleic acid or protein components.

“Treating” as used herein refers to ameliorating at least one symptom of, curing and/or preventing the development of a given disease or condition.

### GENERAL DEFINITIONS

The following definitions are used, unless otherwise described. Alkyl, alkoxy, alkenyl, alkynyl, etc. denote both straight and branched groups; but reference to an individual radical such as propyl embraces only the straight chain radical, a branched chain isomer such as isopropyl being specifically referred to. Aryl denotes a phenyl radical or an ortho-fused bicyclic carbocyclic radical having about nine to ten ring atoms in which at least one ring is aromatic. Heteroaryl encompasses a radical of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms comprising one to four heteroatoms each selected from the group consisting of non-peroxide oxygen, sulfur, and N(X).

The term saccharide includes monosaccharides, disaccharides, trisaccharides and polysaccharides. The term includes glucose, sucrose fructose and ribose, as well as deoxy sugars such as deoxyribose and the like. Saccharide derivatives can conveniently be prepared as described in International Patent Applications Publication Numbers WO 96/34005 and 97/03995. A saccharide can conveniently be linked to the remainder of a compound of formula I through an ether bond.

The invention will now be illustrated by the following non-limiting Examples.

### Example 1

### Synthesis and In Vitro Testing of New Potent Polyacridine-Melittin Gene Delivery Peptides

The combination of a polyacridine peptide modified with a melittin fusogenic peptide results in a potent gene transfer agent. (Baumhover, N. Anderson, K, Fernandez C., and Rice, K. G. “Synthesis of In Vitro Testing of a Potent New Polyacridine-melittin Gene Delivery Peptides” Bioconjugate Chemistry, (2010) 21, 74-83.) Polyacridine peptides of the general formula (Acr-X)ₙ-Cys (SEQ ID NO: 65) were prepared by solid phase peptide synthesis, where Acr is Lys modified on its 8-amine with acridine, X is Arg, Leu or Lys and n is 2, 3 or 4 repeats. The Cys residue was modified by either a maleimide-melittin or a thiolpyridine-Cys-melittin fusogenic peptide resulting in reducible or non-reducible polyacridine-melittin peptides. Hemolysis assays established that polyacridine-melittin peptides retained their membrane lytic potency relative to melittin at pH 7.4 and 5. When combined with plasmid DNA, the membrane lytic potency of polyacridine-melittin peptides was neutralized. Gene transfer experiments in multiple cell lines established polyacridine-melittin peptides mediate expression as efficiently as PEI. The expression was very dependent upon a disulfide bond linking polyacridine to melittin. The gene transfer was most efficient when X is Arg and n is 3 or 4 repeats. These studies establish polyacridine peptides as a novel DNA binding anchor peptide.

An important element of many polyplex non-viral gene delivery systems is a releasable fusogenic peptide that can affect endosomal lysis. The endosomal escape of DNA is especially essential for receptor-targeted gene delivery systems to avoid polyplex degradation by the lysosome.

To improve the endosomal escape of DNA, researchers have incorporated fusogenic peptides, including GALA, KALA (SEQ ID NO: 66), HA2, JTS-1 and melittin, into a non-viral gene delivery system. Most applications in gene delivery have utilized melittin because of its relative ease of chemical synthesis and because its mechanism of membrane lysis has been extensively studied. Melittin has been covalently attached to polylysine, PEI, cationic lipids and even self-polymerized by terminal disulfide bonds and demonstrated to increase in vitro gene transfer efficiency.

While DNA anchoring polymers such as PEI, polylysine, dendrimers and chitosan are known to bind ionically to the phosphate backbone of plasmid DNA, this strategy leads to the formation of cationic polyplexes that favor in vitro gene transfer but to a much lesser degree in vivo gene transfer. This is partly because positively charged polyplexes bind non-specifically to proteins and cells. Attempts at masking the charge with PEG still results in a residual positive charge as measured by zeta potential. The non-specific binding of PEGylated cation polyplexes in vivo complicates strategies that attempt to combine a receptor ligand and releasable fusogenic peptide into a non-viral delivery system. Consequently, it would be beneficial to reversibly bind fusogenic peptides, receptor ligand and PEG to DNA while simultaneously controlling the charge of polyplexes. Likewise, it would be advantageous if the DNA binding anchor were sufficiently small in size to allow for its chemical manipulation and specific functionalization with PEG, targeting ligand and fusogenic peptide.

Polyacridine peptides can reversibly bind with high affinity by intercalating into double stranded DNA. It is possible to synthesize a divalent acridine neoglycopeptide for delivery of DNA to the cells expressing the asialoglycoprotein receptor. Further, polyacridine-nuclear localization sequences (NLS) possessing up to three acridines were shown to improve in vitro gene transfer (Shiraishi, T., Hamzavi, R., and Nielsen, P. E. (2005) Targeted Delivery of Plasmid DNA into the Nucleus of Cells via Nuclear Localization Signal Peptide Conjugated to DNA Intercalating Bis- and Trisacridines Bioconjugate Chemistry 16, 1112-1116).

However to date, a polyacridine DNA binding peptide has not been substituted with a fusogenic peptide. The new strategy described herein demonstrates the synthesis of polyacridine-melittin as a DNA binding peptide and establish its efficacy as a potent in vitro gene transfer agent. The results demonstrate that short polyacridine anchor peptides bind to plasmid DNA with sufficient affinity to deliver melittin in vitro and have the potential to control DNA polyplex charge to allow improved in vivo gene delivery.

Materials and Methods

Unsubstituted Wang resin for peptide synthesis, 9-hydroxybenzotriazole, Fmoc-protected amino acids, O-(Benzotriazole-N,N,N′,N′-tetramethyluronium hexafluorophosphate (HBTU), 1,3-Diisopropylcarbodiimide (DIC), Fmoc-Lysine-OH, and N-Methyl-2-pyrrolidinone (NMP) were obtained from Advanced ChemTech (Lexington, Ky.). N,N-Dimethylformamide (DMF), trifluoroacetic acid (TFA), and acetonitrile were purchased from Fisher Scientific (Pittsburgh, Pa.). Diisopropylethylamine, piperidine, acetic anhydride, Tris(2-carboxyethyl)-phosphine hydrochloride (TCEP), 9-chloroacridine, maleic anhydride, 2-2′ dithiodipyridine (DTDP), and thiazole orange were obtained from Sigma Chemical Co. (St. Louis, Mo.). Polyethylene amine (PEI 25 KDa) was purchased from Aldrich (Milwaukee, Wis.). D-Luciferin and luciferase from Photinus pyralis were obtained from Roche Applied Science (Indianapolis, Ind.). HepG2, CHO, and 3T3 cells were acquired from the American Type Culture Collection (Manassas, Va.). Inactivated qualified fetal bovine serum (FBS) was from Life Technologies, Inc. (Carlsbad, Calif.). BCA reagent was purchased from Pierce (Rockford, Ill.). pGL3 control vector, a 5.3 kb luciferase plasmid containing a SV40 promoter and enhancer, was obtained from Promega (Madison, Wis.). pGL3 was amplified in a DH5α strain of Escherichia coli and purified according to manufacturer's instructions.

Synthesis of 9-phenoxyacridine and Fmoc Lysine(Acridine)

9-phenoxyacridine was synthesized with modification from the methods of Tung et al. (Boulanger, C., Di Giorgio, C., and Vierling, P. (2005) Synthesis of acridine-nuclear localization signal (NLS) conjugates and evaluation of their impact on lipoplex and polyplex-based transfection. Eur J Med Chem 40, 1295-306.) Briefly, 12 g of phenol (127.5 mmol) and 0.72 g of sodium hydroxide (18 mmol) were heated to 100° C. To the liquified phenol, 2.8 g of 9-chloroacridine (13.105 mmol) was added and stirred vigorously for 1.5 hrs. The reaction was quenched by the addition of 100 ml of 2 M sodium hydroxide then allowed to sit at RT overnight. A yellow precipitate was filtered, washed with water and dried in vacuo (3.4822 g, 12.835 mmoles, 97.9%, M.P. 123-124° C., TLC, 15:5:1:0.5, ethyl acetate/methanol/hexane/acetic acid, R_{f}=0.18). ¹H NMR (DMSO-d₆) δ 8.23 (d, 2H), 8.04 (d, 2H), 7.88 (t, 2H), 7.59 (t, 2H), 7.32 (t, 2H), 7.08 (t, 1H), 6.88 (d 2H).

Fmoc-Lysine(Acridine)-OH was prepared by adding 2.18 g of Fmoc-Lys-OH (5.91 mmol) in liquid phenol (6.781 g, 73.01 mmol) to 9-phenoxyacridine (3 g, 11.06 mmol) then heated at 60° C. for 4 hrs under an argon atmosphere. Diethyl ether (80 ml) was then added while stirring vigorously until a yellow precipitate formed that was immediately recovered by filtration and washed repeatedly with diethyl ether. The product was allowed to dry overnight under vacuum (2.90 g, 5.32 mmol, 90%), M.P. 135-140° C., TLC: 1:1, 0.1 v/v % TFA/acetonitrile, R_{f}=0.75). MS: (M+H⁺)¹⁺=545.5 m/z. ¹H NMR (DMSO-d₆) δ 6.7-7.8 (m, 17H), 3.6-3.95 (m, 2H), 3.25-3.53 (br, 3H), 2.06 (m, 1H), 0.8-1.5 (m, 6H).

Synthesis of Maleimide Glycine (Mal-Gly-OH)

Glycine (5 g, 66.6 mmol) and maleic anhydride (6.6 g, 66.6 mmol) were suspended in 80 ml of acetic acid and allowed to react for 3 hrs at RT. The resulting white precipitate was collected by filtration, washed with cold water, and dried (10.95 g, 63.3 mmol, 95%, M.P. 187-189° C., TLC: 2:1:1:1 isopropyl alcohol/acetic acid/ethyl acetate/water, R_{f}=0.5). The glycine maleic acid intermediate was characterized by proton NMR (300 MHz, DMSO-d₆): δ=9.2 (s, 1H) 6.397 (d, 1H, j=8.57 Hz), 6.310 (d, 2H, j=12.86 Hz), 2.0 (d, 2H j=6.86 Hz).

Glycine maleamic acid (5.2 g, 30.04 mmol) and 2.1 equivalents of triethylamine (6.37 g, 63 mmol) were refluxed for 3 hrs in 500 ml toluene with removal of water with a Dean-Stark apparatus. Upon reaction completion, the toluene solution was decanted and dried. The resulting solid was acidified with 2 M HCl, extracted with ethyl acetate, dried with MgSO₄ and evaporated to yield Mal-Gly-OH (1.7 g, 11 mmol, 36.5%, MP 99-110° C., TLC: 2:1:1:1 isopropyl alcohol/acetic acid/ethyl acetate/water, R_{f}=0.72). The product was characterized by proton NMR (300 MHz, DMSO-d₆): δ=7.108 (s, 2H), 4.105 (s, 2H).

Synthesis and Characterization of Polyacridine-Melittin Analogues

Melittin analogues, polyacridine peptides, and control peptides were prepared by solid phase peptide synthesis using standard Fmoc procedures with 9-hydroxybenzotriazole and HBTU double couplings on a 30 μmol scale on an Advanced ChemTech APEX 396 synthesizer. Mal-melittin (SEQ ID NO: 67) was prepared by coupling Mal-Gly-OH to the N-terminus of side-chain protected full-length melittin on resin utilizing a 6-fold excess of Mal-Gly-OH, DIC and HOBt, reacted for 4 hrs while stirring. The resin was filtered, washed with DMF, DCM and methanol, and then dried. Peptides were removed from resin and side chain deprotected using a cleavage cocktail of TFA/triisoproylsilane/water (95:2.5:2.5 v/v/v) for 3 hrs followed by precipitation in cold ether. Precipitates were centrifuged for 10 min at 4000 rpm at 4° C. and the supernatant decanted. Peptides were then reconstituted with 0.1 v/v % TFA and purified to homogeneity on RP-HPLC by injecting 0.5-2 μmol onto a Vydac C18 semipreparative column (2×25 cm) eluted at 10 ml/min with 0.1 v/v % TFA with an acetonitrile gradient of 20-45 v/v % over 30 min while monitoring tryptophan (Abs 280 nm) or acridine (Abs 409 nm). The major peak was collected and pooled from multiple runs, concentrated by rotary evaporation, lyophilized, and stored at −20° C. Purified peptides were reconstituted in 0.1% v/v TFA and quantified by absorbance (tryptophan ε_{280 nm}=5600 M⁻¹ cm⁻¹, thiolpyridine and tryptophan ε_{280 nm}=10915 M⁻¹ cm⁻¹, or acridine ε_{409 nm}=9266 M⁻¹ cm⁻¹) to determine isolated yield. Purified peptides were characterized by LC-MS by injecting 2 nmol onto a Vydac C18 analytical column (0.47×25 cm) eluted at 0.7 ml/min with 0.1 v/v % TFA and an acetonitrile gradient of 10-55 v/v % over 30 min while acquiring ESI-MS in the positive ion mode.

Cys-melittin (SEQ ID NO: 52) was reacted with 10-eq of DTDP in 2-propanol/2 N Acetic Acid (10:3) for 8 hrs at RT to generate the thiolpyridine protected peptide. The reaction mixture was concentrated by rotary evaporation and applied to a Sephadex G-10 column eluted (2.5×50 cm) with 0.1 v/v TFA while monitoring absorbance at 280 nm to remove excess DTDP. The peptide peak fractions were pooled, concentrated, lyophilized, and purified to homogeneity by RP-HPLC as described previously.

Poly(Acr-X)ₙ-SS-melittin and poly(Acr-X)ₙ-Mal-melittin peptides were synthesized on a 2 μmol scale based on melittin, using 1.7 equivalents (3.4 μmol) of the reduced poly(Acr-X)ₙ-Cys, where X equals Arg, Lys, or Leu. Mal-melittin (SEQ ID NO: 67) (2 μmol) was prepared in 8 ml of 10 mM ammonium acetate pH 5 and added to poly(Acr-X)ₙ-Cys and 2 ml of methanol to facilitate solubility. The reaction occurred over 12 hrs at RT as determined by analyzing aliquots by LC-MS. At completion the reaction mixture was concentrated by rotary evaporation and lyophilized prior to purification of poly(Acr-X)ₙ-Mal-melittin by RP-HPLC eluted with 0.1 v/v % TFA and acetonitrile gradient (35-45% over 30 min) while detecting at 409 nm.

Alternatively, thiolpyridine-Cys-melittin (2 μmol) was prepared in 8 ml of 10 mM ammonium acetate pH 6 and added to 3.4 vμmol poly(Acr-X)ₙ-Cys in 2 ml of methanol. After 24 hrs, poly(Acr-X)ₙ-SS-melittin was purified and characterized by LC-MS as described above.

Polyacridine-Melittin Hemolysis Assay

Whole blood was obtained from male ICR mice by heart puncture with heparinized 22 G needles and collected in conical tubes containing 10 ml of 0.15 M PBS (pH 7.4) prewarmed to 37° C. Erythrocytes were immediately separated from plasma by centrifugation at 2000 rpm for 2 min, washed three times with 10 ml of PBS, and then diluted to 1.5×10⁸ cells/ml. Peptides were prepared at 15 μM and serially diluted to 10-0.01 μM, after which 100 μl was pipetted in triplicate into a MultiScreenHTS BV 96 well plate. Erythrocytes (50 μl, 7.5×10⁶ cells) were added to the peptides and incubated at 37° C. for 1 h followed by filtration on a Multiscreen vacuum manifold (Millipore Corporation, Billerica, Mass.). The filtrate was measured for Abs₄₁₀ₙₘ on a Biotech plate reader (Biotech Instruments Incorporated., Winooski, Vt.) and compared to the 100% hemolysis caused by replacing PBS with water. Peptide DNA polyplexes (1.5 nmols and 7.5 μg pGL3) were also assayed for hemolysis as described above.

Formulation and Characterization of Polyacridine-Melittin Polyplexes

The relative binding affinity of peptides for DNA was determined by a fluorophore exclusion assay (Rich, D. H., Gesellchen, P. D., Tong, A., Cheung, A., and Buckner, C. K. (1975) Alkylating derivatives of amino acids and peptides. Synthesis of N-maleoylamino acids, [1-(N-maleoylglycyl)cysteinyl]oxytocin, and [1-(N-maleoyl-11-aminoundecanoyl)cysteinyl]oxytocin. Effects on vasopressin-stimulated water loss from isolated toad bladder. J. Med. Chem. 18, 1004-1010). pGL3 (200 μl of 4 μg/ml in 5 mM Hepes pH 7.5 containing 0.1 μM thiazole orange) was combined with 0, 0.05, 0.1, 0.25, 0.35, 0.5, 1, and 2 nmols of peptide in 300 μl of Hepes and allowed to bind at RT for 30 min. Thiazole orange fluorescence was measured using an LS50B fluorometer (Perkin-Elmer, U.K.) by exciting at 498 nm while monitoring emission at 546 nm with the slit widths set at 10 nm. A fluorescence blank of thiazole orange in the absence of DNA was subtracted from all values before data analysis. The data is presented as nmol of peptide per μg of DNA versus the percent fluorescence intensity±the standard deviation determined by three independent measurements.

Particle size and zeta potential were determined by preparing 2 ml of polyplex in 5 mM Hepes pH 7.5 at a DNA concentration of 30 μg/ml with 15 nmols/ml of peptide (0.5 nmol of peptide per μg of DNA). Particle size was measured by quasi-elastic light scattering (QELS) at a scatter angle of 90° on a Brookhaven ZetaPlus particle sizer (Brookhaven Instruments Corporation, NY). The zeta potential was determined as the mean of ten measurements immediately following acquisition of the particle size.

Polyacridine-melittin DNA polyplexes were prepared at 0.5 nmol of peptide per μg of DNA at a concentration of 100 μg per ml of DNA and directly deposited on a freshly cleaved mica surface and allowed to bind for 10 min prior to washing with deionized water. Alternatively, naked DNA (100 μg pGL3 per ml) was prepared in 10 mM Tris, 1 mM EDTA pH 8, then diluted to 1 μg per ml in 40 mM Hepes 5 mM nickel chloride pH 6.7, and deposited on a fresh cleaved mica surface for 10 min followed by washing with deionized water. An Asylum atomic force microscope (AFM) MFP3D (Santa Barbara, Calif.) was operated in the AC-mode in order to image either naked DNA or DNA polyplexes using a silicon cantilever (Ultrasharp NSC15/AIBS, MikroMasch).

Polyacridine-Melittin Polyplex Toxicity

The in vitro toxicity of polyacridine-melittin polyplexes was evaluated by MTT assay (Wadhwa, M. S., Collard, W. T., Adami, R. C., McKenzie, D. L., and Rice, K. G. (1997) Peptide-mediated gene delivery: influence of peptide structure on gene expression. Bioconjugate Chemistry 8, 81-8). CHO, HepG2 and 3T3 cells were plated on 6×35 mm wells at 5×10⁵ cells/well and grown to 40-70% confluency. The media was replaced with 2 ml of fresh MEM supplemented with 2% FBS, and the cells treated with 0.5 nmol peptide/μg DNA polyplexes. After 6 hrs, the media was replaced with fresh culture media and grown an additional 18 hrs. The media was replaced with 2 ml of fresh media and 500 μL of 0.5% (w/v) 3-(4,5-dimethylthiazole-2-yl)-2,5-diphenyl tetrazolium bromide (MTT) in PBS solution and allowed to incubate at 37° C. for 1 hr to let formazan crystals form. The media containing MTT was removed, and the crystals dissolved by the addition of 2 mL of dimethyl sulfoxide (DMSO) and 250 μL Sorenson's Glycine Buffer, then measured by absorbance 595 nm on a microplate reader. The percent viability was determined relative to untreated cells.

In Vitro Gene Transfer of Polyacridine-Melittin DNA Polyplexes

HepG2 cells (5×10⁵) were plated on 6×35 mm wells and grown to approximately 50% confluency. Transfections were performed in MEM supplemented with 2% FBS, sodium pyruvate (1 mM), and penicillin and streptomycin (100 U and 100 μg/mL). Polyplexes were prepared at a DNA concentration of 30 μg/ml and a stoichiometry of 0.5 nmol peptide per μg of DNA in Hepes buffered mannitol (HBM). Polyplexes (10 μg of DNA in 0.3 ml of HBM) were added drop wise to wells in triplicate. After 24 hrs, the cells were washed twice with 2 ml of ice-cold phosphate-buffered saline (Ca²⁺- and Mg²⁺-free) and then treated with 0.5 ml of lysis buffer (25 mM Tris Chloride, pH 7.8, 1 mM EDTA, 8 mM magnesium chloride, and 1% Triton X-100) for 10 min at 4° C. Cell lysates were scraped, transferred to 1.5 mL microcentrifuge tubes, and centrifuged for 10 min at 13,000 g at 4° C. to pellet cell debris. Lysis buffer (300 μL), sodium-ATP (4.3 μL of a 165 mM solution at pH 7, 4° C.) were combined in a test tube, mixed briefly, and immediately placed in the luminometer. Luciferase relative light units were measured by a Lumat LB 9501 (Berthold Systems, Germany) with 10 s integration after automatic injection of 100 μL of 0.5 mM D-luciferin. The relative light units were converted to fmol using a standard curve generated by adding a known amount of luciferase to 35 mm wells containing 50% confluent HepG2 cells. The resulting standard curve had an average slope of 2.6×10⁴ relative light units/fmol enzyme. Protein concentrations were measured by a BCA assay using bovine serum albumin as a standard. The amount of luciferase recovered in each sample was normalized to mg of protein and reported as the mean and standard deviation obtained from triplicate transfections. PEI pGL3 polyplexes were prepared by mixing 35 μg of DNA in 525 μL of HBM with 43.8 μg PEI in 525 μL of HBM while vortexing to create DNA complexes possessing a charge ratio (NH₄⁺/PO₄⁻) of 9:1. Cells were transfected with 10 μg PEI-DNA polyplexes as previously described. CHO and 3T3 (5×10⁵) cells were plated on 6×35 mm wells and grown to ca. 50% confluency and then transfected as described above.

Results

Synthetic Strategy for Polyacridine-Melittin

Naturally occurring melittin isolated from bee venom is a 26 amino acid peptide amide composed of two a-helices conjoined through an interrupting proline. Numerous studies have been conducted to investigate the mechanism and sequence specificity of melittin's membrane lytic activity.

Natural melittin contains a single aromatic Trp at residue 8 and does not contain a Cys residue. To improve the synthesis of polyacridine-melittin, we replaced Trp 8 with Leu, and reinstalled a single Trp near the N-terminus in Cys-Trp-Lys-Lys (SEQ ID NO: 51). The reactivity of the Cys residue was increased by flanking Lys residues. This also allowed selective chromatographic detection of full length melittin at 280 nm. These modifications resulted in a Cys-melittin analogue: CWKKGIGAVLKVLTTGLPALISLIKRKRQQ (SEQ ID NO: 52), that SOPMA analysis predicted to maintain a-helical character. A similar Mal-melittin analogue (Mal-GWKKGIGAVLKVLTTGLPALISLIKRKRQQ (SEQ ID NO: 67)), possessing an N-terminal maleimide-glycine (Mal-G) in place of Cys, was also prepared.

Polyacridine and melittin peptides were synthesized using HBTU/HOBt with increased coupling efficiency compared to DIC. Early attempts to synthesize polyacridine peptides using DIC resulted in heterogeneous mixtures of truncated peptides with minimal recovery of full length peptide. The identity of the spacing amino acid between Lys-acridine residues significantly influenced the synthetic yield. Bulky amino acid side chains and protecting groups such as Lys-Boc, Phe-Trt, Leu, Glu-OBut and Arg-Pbf were well tolerated, whereas Gly surprisingly was not. Polyacridine peptides possess a C-terminal Cys to accommodate bioconjugation to Cys-melittin (SEQ ID NO: 52) or Mal-melittin (SEQ ID NO: 67). Polyacridine peptides possessing an alternating Lys-Acr (Acr) spaced by either Arg, Lys, or Leu were designed to influence the charge character of the anchor peptide (Scheme 1).

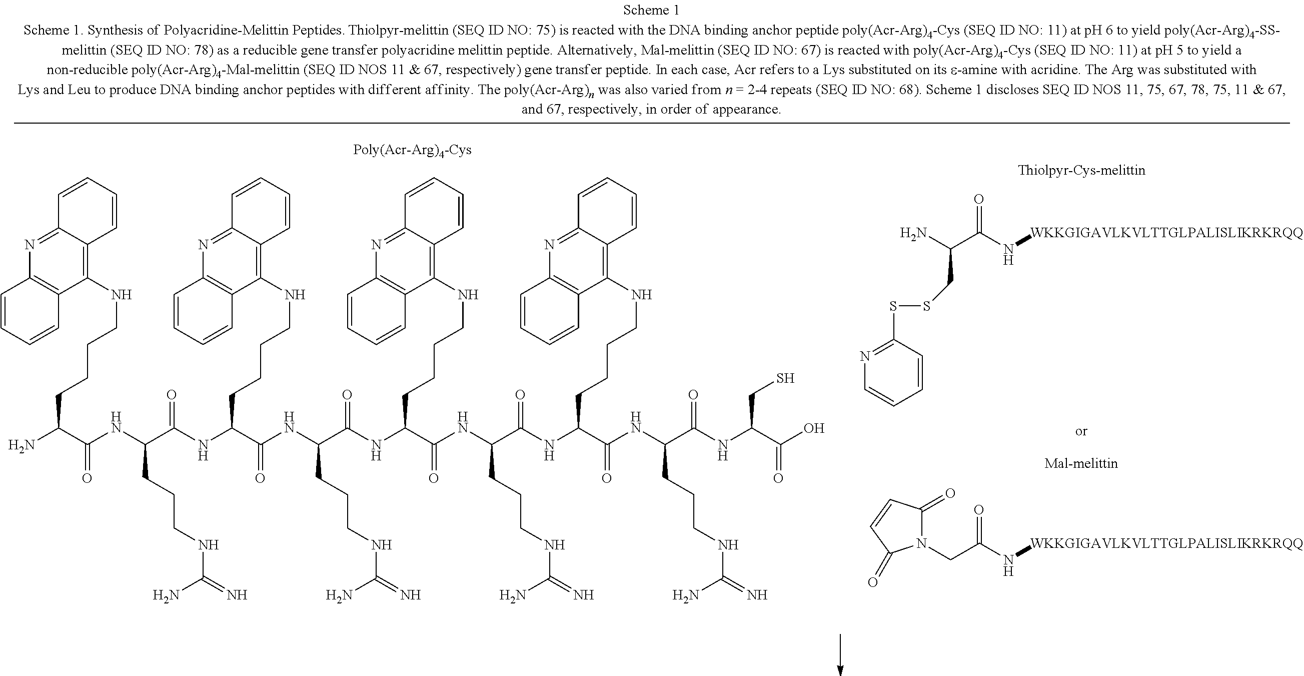

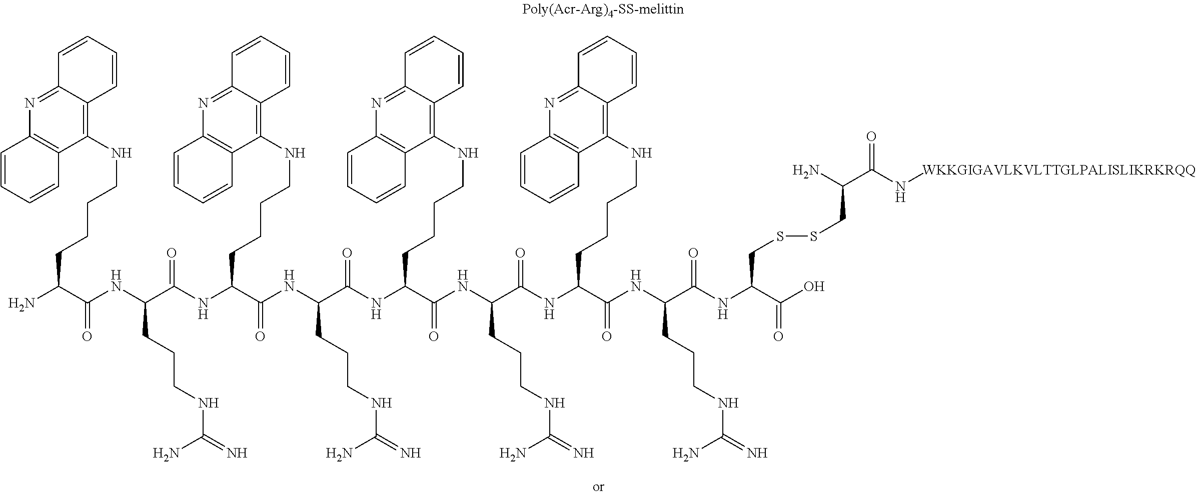

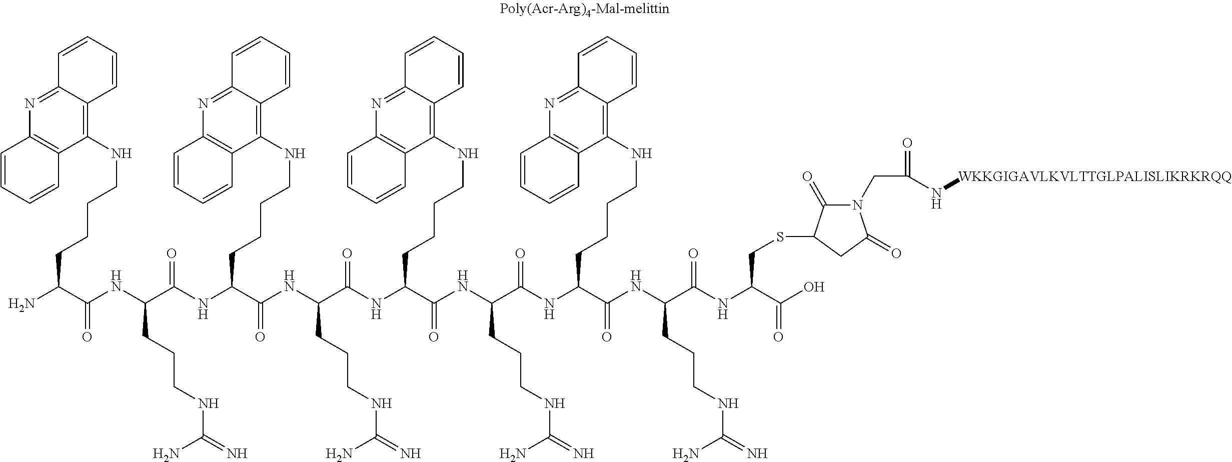

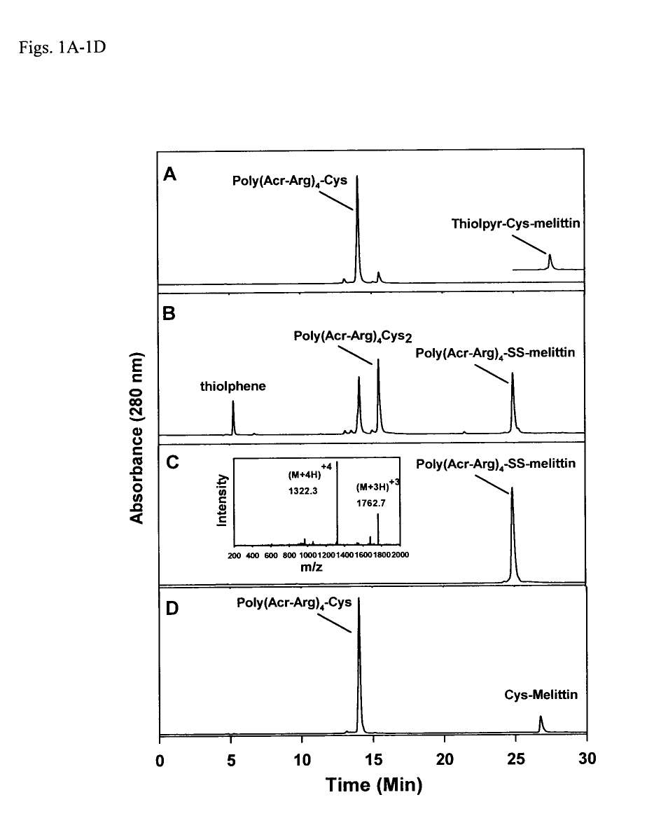

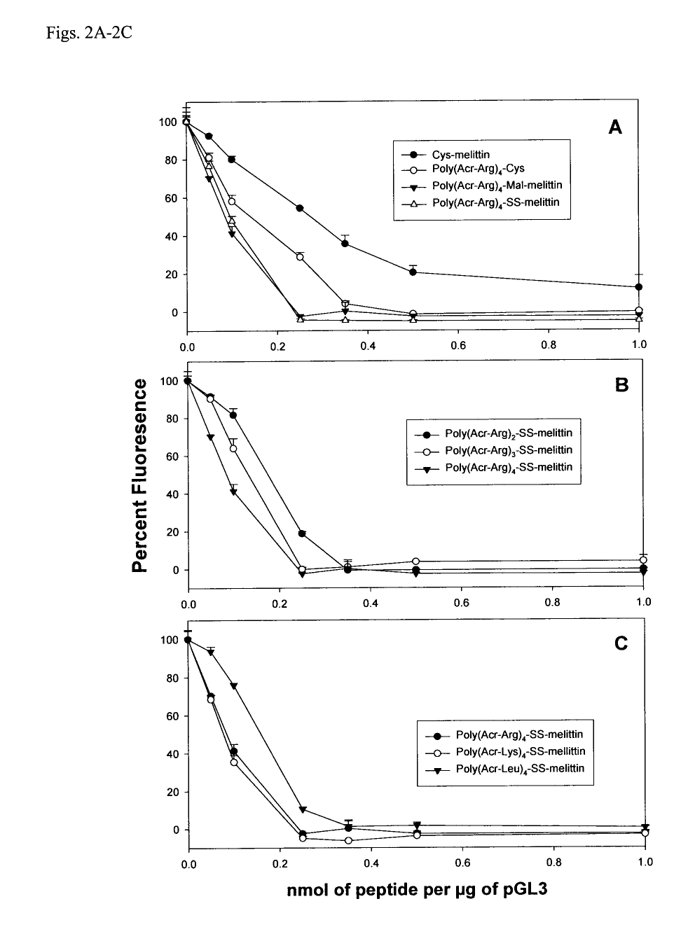

The length of polyacridine peptides was systematically varied to possess 2-4 Acr-Arg repeats to examine the influence of affinity. Polyacridine nona peptides were routinely obtained in 30-35% purified yield with >95% purity. Polyacridine-melittin peptides were designed to possess either a reducible dithiol or a non-reducible maleimide linkage (Scheme 1). The most expedient synthetic route proved to be cleaveage of Cys-melittin (SEQ ID NO: 52) from the resin, followed by modification with dithiol dipyridine to produce a thiolpyr-melittin (SEQ ID NO: 75). The RP-HPLC monitoring of the reaction of thiolpyr-melittin (SEQ ID NO: 75) with poly(Acr-Arg)₄-Cys (SEQ ID NO: 11) is illustrated in FIGS. 1A and B. The desired poly(Acr-Arg)₄-SS-melittin (SEQ ID NO: 78) product is formed with complete consumption of thiolpyr-melittin (SEQ ID NO: 75), along with formation of a poly(Acr-Arg)₄-Cys₂ (SEQ ID NO: 12) by-product (FIG. 2B). This synthetic route resulted in poly(Acr-X)ₙ-SS-melittin in 35-50% purified yield at >95% purity as illustrated by LC-MS analysis of purified poly(Acr-Arg)₄-SS-melittin (SEQ ID NO: 78) (FIG. 1C). ESI-MS was used to establish structure based on the triply and quadruplely charged positive ions corresponding to the calculated mass of the desired polyacridine-melittin peptide (FIG. 1C, inset). The release of melittin from poly(Acr-Arg)₄-SS-melittin (SEQ ID NO: 78) was established by reaction with TCEP to regenerate poly(Acr-Arg)₄-Cys (SEQ ID NO: 11) and Cys-melittin (SEQ ID NO: 52) peptide peaks (FIG. 1D).

Poly(Acr-X)₄-Mal-melittin peptides, where X is Lys (SEQ ID NOS 71 & 67, respectively) or Leu (SEQ ID NOS 72 & 67, respectively), were synthesized to compare their gene transfer properties with poly(Acr-Arg)₄-SS-melittin (SEQ ID NO: 78). The synthetic strategy utilized Gly-maleimide that was coupled to the N-terminus of the fully protected melittin peptide on resin (Scheme 1). On work-up this yielded Mal-melittin (SEQ ID NO: 67) that was then directly conjugated to poly(Acr-X)ₙ-Cys peptides. The conjugation of Mal-melittin (SEQ ID NO: 67) with poly(Acr-Arg)₄-Cys (SEQ ID NO: 11) was monitored by RP-HPLC resulting in the complete consumption of Mal-melittin (SEQ ID NO: 67) and the formation of poly(Acr-Arg)₄-Mal-melittin (SEQ ID NOS 11 & 67, respectively). Purification of poly(Acr-Arg)₄-Mal-melittin (SEQ ID NOS 11 & 67, respectively) was achieved with a final yield of 35% and >95% purity. Reducible and non-reducible polyacridine-melittin peptides with Acr-Arg repeat of 2 and 3 were synthesized with similar yield and purity, and characterized by LC-MS (Table 1).

TABLE 1
Sequence and Hemolytic Potency of Peptide Conjugates.

| | Mass^{a} | HL50 (μM)^{b} |
|---|---|---|
| | (calc/obs) | pH 7.4:5.0 |
| Polyacridine Anchor Peptides (Acr) = Lvs-ε-acridine, Ac = acylated ε-amine) | | |
| poly(Acr-Arg)₂-Cys (SEQ ID NO: 69) | 1044.3/1044.2 | N.D.^{c} |
| poly(Acr-Arg)₃-Cys (SEQ ID NO: 70) | 1505.8/1505.6 | N.D. |
| poly(Acr-Arg)₄-Cys (SEQ ID NO: 11) | 1967.4/1967.0 | >10/>10 |
| poly(Acr-Lys)₄-Cys (SEQ ID NO: 71) | 1855.3/1855.0 | N.D. |
| poly(Acr-Leu)₄-Cys (SEQ ID NO: 72) | 1795.3/1795.1 | N.D. |
| Trp-(Lys(Ac)-Arg)₄-Cys (SEQ ID NO: 73) | 1612.9/1612.7 | N.D. |
| Melittin Analogues (thiolpyr = thiol pyridine, Mal = maleimide) | | |
| Melittin GIGAVLKVLTTGLPALISWIKRKRQQ (SEQ ID NO: 50) | 2847.5/2847.4 | 0.9/2.3 |
| Cys-melittin CWKKGIGAVLKVLTTGLPALISLIKRKRQQ (SEQ ID NO: 52) | 3320.1/3320.4 | 1.0/0.7 |
| Dimeric-melittin (CWKKGIGAVLKVLTTGLPALISLIKRKRQQ)₂ | 6638.2/6637.6 | 1.0/1.2 |
| (Core sequence disclosed as SEQ ID NO: 74) | | |
| Thiolpyr-melittin C(tp)WKKGIGAVLKVLTTGLPALISLIKRKRQQ (SEQ ID NO: 75) | 3429.2/3428.4 | N.D. |
| Mal-melittin (m)GWKKGIGAVLKVLTTGLPALISLIKRKRQQ (SEQ ID NO: 67) | 3354.0/3454.0 | 3.84/4.8 |
| Polyacridine-Melittin (SS = reducible, Mal = non-reducible) | | |
| poly(Acr-Arg)₂-SS-melittin (Acr-Arg)₂-C-CWKKGIGAVLKVLTTGLPALISLIKRKRQQ | 4359.5/4362.3 | 2.5/3.9 |
| (SEQ ID NO: 76) | | |
| poly(Acr-Arg)₃-SS-melittin (Acr-Arg)₃-C-CWKKGIGAVLKVLTTGLPALISLIKRKRQQ | 4823.9/4823.2 | 1.4/2.2 |
| (SEQ ID NO: 77) | | |
| poly(Acr-Arg)₄-SS-melittin (Acr-Arg)₄-C-CWKKGIGAVLKVLTTGLPALISLIKRKRQQ | 5285.5/5285.2 | 1.7/1.9 |
| (SEQ ID NO: 78) | | |
| poly(Acr-Arg)₄-Mal-melittin (Acr-Arg)₄-C(m)GWKKGIGAVLKVLTTGLPALISLIKRKRQQ | 5321.4/5320.8 | 2.1/1.7 |
| (SEQ ID NOS 11 & 67, respectively) | | |
| poly(Lys(Ac)-Arg)₄-SS-melittin W(Lys(Ac)-Arg)₄-C-CWKKGIGAVLKVLTTGLPALISLI | 4931.0/4930.4 | 5.6/7.7 |
| KRKRQQ (SEQ ID NO: 79) | | |
| poly(Acr-Lys)₄-SS-melittin (Acr-Lys)₄-C-CWK KG IGAVLKVLTTGLPALISLIKRKRQQ | 5173.4/5173.2 | 1.0/2.8 |
| (SEQ ID NO: 80) | | |
| poly(Acr-Lys)₄-Mal-melittin (Acr-Lys)₄-C(m)GWKKGIGAVLKVLTTGLPALISLIKRKRQQ | 5209.4/5208.8 | 1.5/1.9 |
| (SEQ ID NOS 71 & 67, respectively) | | |
| poly(Acr-Leu)₄-SS-melittin (Acr-Leu)₄-C-CWK KG IGAVLKVLTTGLPALISLIKRKRQQ | 5113.4/5112.8 | 2.2/1.6 |
| (SEQ ID NO: 81) | | |
| poly(Acr-Leu)₄-Mal-melittin (Acr-Leu)₄-C(m)GWKKGIGAVLKVLTTGLPALISLIKRKRQQ | 5149.3/5149.2 | 2.4/1.2 |
| (SEQ ID NOS 72 & 67, respectively) | | |

^{a}Calculated and observed mass as determined by ESI LC-MS.
^{b}RBC hemolysis at pH 7.4 and 5.0.
^{c}N.D. = not determined

Physical Properties of Polyacridine-Melittin DNA Polyplexes

The binding of polyacridine-melittin peptides to pGL3 was investigated using a thiazole orange exclusion assay (Rich, D. H., Geselichen, P. D., Tong, A., Cheung, A., and Buckner, C. K. (1975) Alkylating derivatives of amino acids and peptides. Synthesis of N-maleoylamino acids, [1-(N-maleoylglycyl)cysteinyl]oxytocin, and [1-(N-maleoyl-11-aminoundecanoyl)cysteinyl]oxytocin. Effects on vasopressin-stimulated water loss from isolated toad bladder. J. Med. Chem. 18, 1004-1010). Polyacridine-melittin peptides were found to compete for intercalation and displace thiazole orange resulting in a decrease in fluorescence intensity. When titrated at increasing concentration the fluorescence decreased until an asymptote is reached at an equivalence point (FIG. 2A). Analysis of the DNA binding affinity of poly(Acr-Arg)₄-Cys (SEQ ID NO: 11) established an equivalence point at approximately 0.4 nmols peptide per μg of DNA. By comparison, Cys-melittin (SEQ ID NO: 52) demonstrated weaker binding and was not able to fully exclude thiazole orange, only approaching an equivalence point at approximately 0.5 nmol of peptide per μg of DNA (FIG. 2A). Alternatively, poly(Acr-Arg)₄-SS-melittin (SEQ ID NO: 78) demonstrated higher affinity resulting in an equivalence point at approximately 0.25 nmols of peptide per μg of DNA. This affinity is comparable to that afforded by a Cys-Trp-Lys₁₈ peptide (SEQ ID NO: 82) using the same assay.

Comparison of poly(Acr-Arg)₂₋₄-SS-melittin (SEQ ID NOS 76-78, respectively) demonstrated nearly equivalent affinity for Acr-Arg repeats of 3 and 4, but less affinity afforded for a repeat of 2 (FIG. 2B). Direct comparison of the DNA binding affinity of poly(Acr-Arg)₄-SS-melittin (SEQ ID NO: 78) with poly(Acr-Lys)₄-SS-melittin (SEQ ID NO: 80) and poly(Acr-Leu)₄-SS-melittin (SEQ ID NO: 81) established there was a negligible difference, with each producing fully complexed polyplexes at 0.3 nmol per μg of pGL3 (FIG. 2C).

The particle size and zeta potential of polyacridine-melittin polyplexes were determined by QELS analysis (Table 2).

TABLE 2
Physical Properties and Toxicity of DNA Polyplexes

| | SEQ ID | Particle Size | Zeta Potential | |
|---|---|---|---|---|
| Peptide | NO: | (nm)^{a} | (+mV)^{b} | MTT Assay^{c} |
| poly(Acr-Arg)₂-SS-melittin | 76 | 737 ± 46.8 | 25 ± 2.2 | 99.2 ± 6.6 |
| poly(Acr-Arg)₃-SS-melittin | 77 | 126 ± 4.5 | 29 ± 4.5 | 102.6 ± 6.5 |
| poly(Acr-Arg)₄-SS-melittin | 78 | 84 ± 2.0 | 29 ± 2.6 | 70.4 ± 5.0 |
| poly(Acr-Arg)₄-Mal-melittin | 11& 67, | 79 ± 1.3 | 28 ± 2.5 | 69.2 ± 5.0 |
| | respectively | | | |
| poly(Lys(Ac)-Arg)₄-SS-melittin | 79 | 177 ± 6.2 | 20 ± 2.9 | 54.6 ± 4.0 |
| poly(Acr-Lys)₄-SS-melittin | 80 | 89 ± 3.5 | 23 ± 1.6 | 75.6 ± 4.7 |
| poly(Acr-Lys)₄-Mal-melittin | 71 & 67, | 86 ± 2.4 | 26 ± 2.0 | 86.6 ± 7.6 |
| | respectively | | | |
| poly(Acr-Leu)₄-SS-melittin | 81 | 119 ± 1.4 | 20 ± 2.9 | 45.6 ± 4.1 |
| poly(Acr-Leu)₄-Mal-melittin | 72 & 67, | 121 ± 2.5 | 22 ± 2.1 | 89.7 ± 3.1 |
| | respectively | | | |

^{a}Mean particle size of peptide-DNA polyplex (0.5 nmol of peptide per μg of DNA) as determined by QELS.
^{b}Mean zeta potential determined in 5 mM Hepes, pH 7.4.
^{c}Percent viability of peptide-DNA polyplexes determined by MTT assay in CHO cells (0.5 nmol of peptide per μg DNA) at 10 μg DNA dose applied to 5 × 10⁵ cells for 6 hours. Nearly identical results were obtained for HepG2 and 3T3 cells.

Poly(Acr-Arg)₄-SS-melittin (SEQ ID NO: 78) and poly(Acr-Arg)₄-Mal-melittin (SEQ ID NOS 11 & 67, respectively) produced polyplexes of 79-84 nm average diameter, respectively. Comparison of the size of polyplexes prepared with poly(Acr-Arg)₃-SS-melittin (SEQ ID NO: 77) (126 nm) and poly(Acr-Arg)₂-SS-melittin (SEQ ID NO: 76) (737 nm) established that shorter repeats produced larger particle size (Table 2). This result is consistent with earlier findings that indicated that shorter, low affinity polylysine peptides (<13 residues) (SEQ ID NO: 84) produced larger polyplexes, whereas polylysine of 18 repeats or longer (SEQ ID NO: 85) produced polyplexes of approximately 80 nm in diameter.

Substitution of the spacing Arg for Lys or Leu in Acr-Arg repeats resulted in a minimal change in particle size (Table 2). This supports the hypothesis that polyintercalation, and not ionic binding, is primarily responsible for the binding affinity to DNA. To test this hypothesis, an analogue was prepared that replaced the acridine modified Lys with an acetyl on the s-amine of lysine resulting in poly(Lys(Ac)-Arg)₄-SS-melittin (SEQ ID NO: 79). This analogue produced polyplexes of average diameter of 177 nm, suggesting a significantly lower affinity (Table 2).

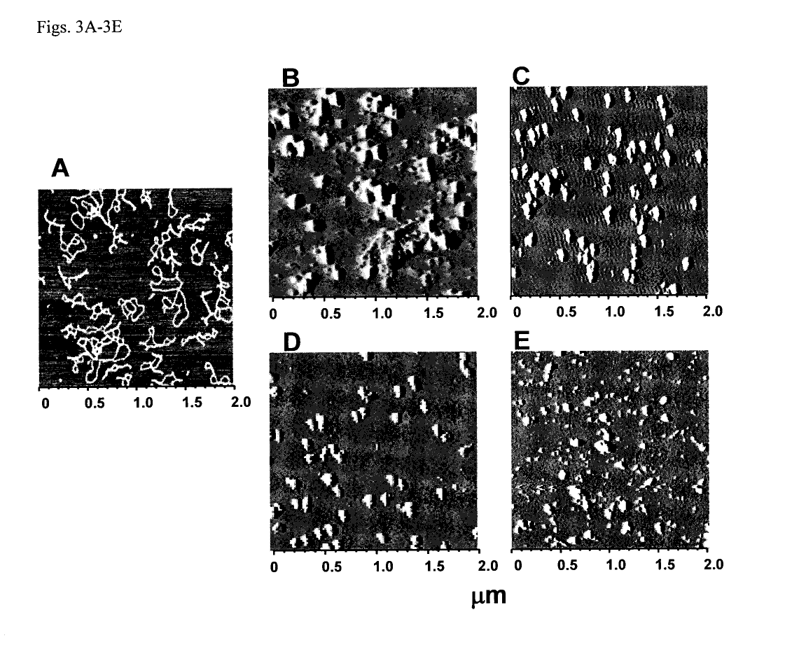

To further validate the change in particle size determined by QELS, polyplexes were analyzed by atomic force microscopy (AFM). Analysis of pGL3 by AFM demonstrated an open structure occupying from 0.5-1 μm with no single morphology (FIG. 3A). Polyplexes prepared with poly(Acr-Arg)₂-SS-melittin (SEQ ID NO: 76) appeared to form clusters composed of several individual particles that account for the larger size determined by QELS (FIG. 3B). In contrast, the AFM images for polyplexes prepared with poly(Acr-Arg)₄-SS-melittin (SEQ ID NO: 78) (FIG. 3C), poly(Acr-Leu)₄-SS-melittin (SEQ ID NO: 81) (FIG. 3D), poly(Acr-Lys)₄-SS-melittin (SEQ ID NO: 80) (FIG. 3E) all appeared to be less aggregated resulting in overall smaller particle size. It is interesting to note that the zeta potentials of polyacridine-melittin polyplexes containing a spacing Arg, Lys or Leu were approximately +25 mV (Table 2). This result indicated that the charge on melittin strongly influenced the overall charge of polyplexes prepared with different anchoring peptides.

Biological Activity of Polyacridine-Melittin DNA Polyplexes

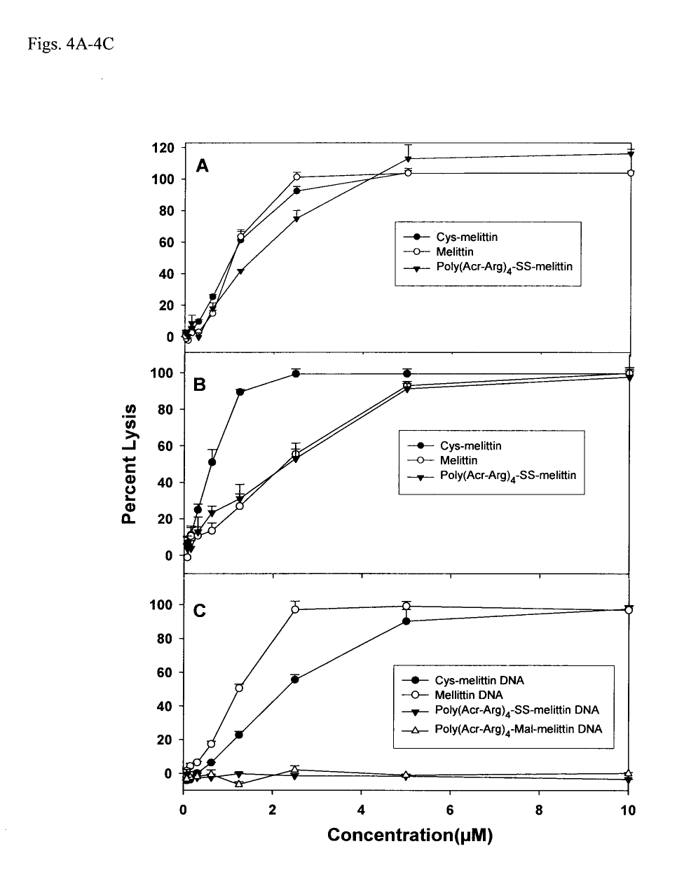

The membrane lytic potency of polyacridine-melittin peptides were investigated using a RBC hemolysis assay (Chen, C.-P., Kim, J.-S., Steenblock, E., Liu, D., and Rice, K. G. (2006) Gene transfer with poly-melittin peptides. Bioconjugate Chemistry 17, 1057-62). As anticipated, poly(Acr-X)₄-Cys (SEQ ID NO: 88) anchor peptides alone were inactive in membrane lysis. Alternatively, modification of the N-terminus of natural melittin with Cys-Trp-Lys-Lys (SEQ ID NO: 51) and substitution of Leu for Trp 8 to generate Cys-melittin (SEQ ID NO: 52), resulted in retention of RBC hemolytic (HL₅₀) potency at pH 7.4 relative to natural melittin (FIG. 4A). Likewise, the hemolytic potency of poly(Acr-Arg)₄-SS-melittin (SEQ ID NO: 78) was comparable to Cys-melittin (SEQ ID NO: 52), indicating the attachment of the polyacridine anchor had a negligible influence on the membrane lytic activity of Cys-melittin (SEQ ID NO: 52). The results for polyacridine-melittin peptides possessing either a reducible or non-reducible linkage are summarized in Table 1, establishing their nearly equivalent hemolytic potency at pH 7.4.

The hemolytic potency of polyacridine melittin peptides was also determined at pH 5 to simulate the pH of the endosome. In contrast to an earlier finding that C and N-terminal modification of natural melittin with Cys-(Lys)₄ (SEQ ID NO: 86) resulted in the complete loss of hemolytic activity at pH 5, Cys-melittin (SEQ ID NO: 52) possessed an improved pH 5 RBC lytic potency relative to that of natural melittin (FIG. 4B). Consequently, poly(Acr-Arg)₄-SS-melittin (SEQ ID NO: 78) and poly(Acr-Arg)₄-Mal-melittin (SEQ ID NOS 11 & 67, respectively) both possess potent hemolytic activity at pH 7.4 and 5 (Table 1). When combined with pGL3, Cys-melittin (SEQ ID NO: 52) and Mal-melittin (SEQ ID NO: 67) maintained their hemolytic activity at pH 7.4, demonstrating their weak association with DNA (FIG. 4C). In contrast, the hemolytic activity of poly(Acr-Arg)₄-SS-melittin (SEQ ID NO: 78) DNA polyplexes was completely neutralized due to the strong binding of the polyacridine peptide with DNA (FIG. 4C). Reduction of the disulfide bond would decrease the binding affinity to DNA and thereby release hemolytic Cys-melittin (SEQ ID NO: 52).

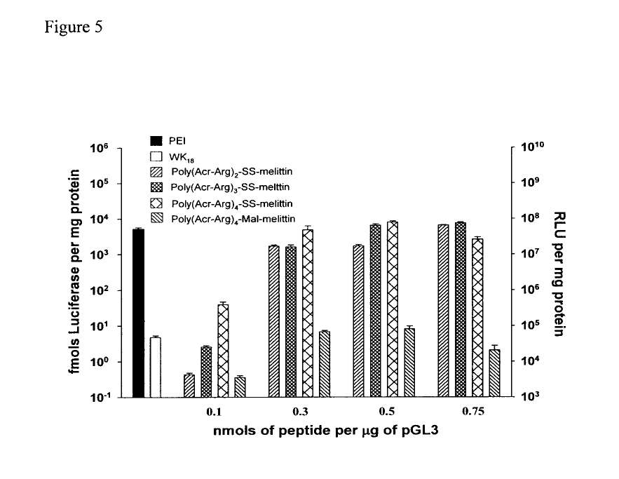

To establish a relationship between peptide anchor length, reducibility, and peptide DNA stoichiometry, CHO cells were transfected with polyplexes formed with poly(Acr-Arg)₂-SS-melittin (SEQ ID NO: 76), poly(Acr-Arg)₃-SS-melittin (SEQ ID NO: 77), poly(Acr-Arg)₄-SS-melittin (SEQ ID NO: 78), and poly(Acr-Arg)₄-Mal-melittin (SEQ ID NOS 11 & 67, respectively) while varying the peptide to DNA ratio from 0.1, 0.35, 0.5, and 0.75 nmols of peptide per μg of DNA (FIG. 5). Luciferase reporter gene expression for polyplexes formed with 0.1 nmol of peptide per μg DNA resulted in expression comparable to a WK₁₈/DNA control polyplex (“WK₁₈” disclosed as SEQ ID NO: 13). Increasing the stoichiometry to 0.35 nmols of peptide per μg of DNA or higher increased the gene expression approximately 100-fold relative to 0.1 nmol of peptide per μg of DNA. Within this series, poly(Acr-Arg)₄-SS-melittin (SEQ ID NO: 78) produced the highest expression at a stoichiometry of 0.3 and 0.5. The expression mediated by poly(Acr-Arg)₄-SS-melittin (SEQ ID NO: 78) was approximately 1000-fold greater than that of poly(Acr-Arg)₄-Mal-melittin (SEQ ID NOS 11 & 67, respectively) which differed only by the reducibility of the linkage between the anchor and melittin (FIG. 5).

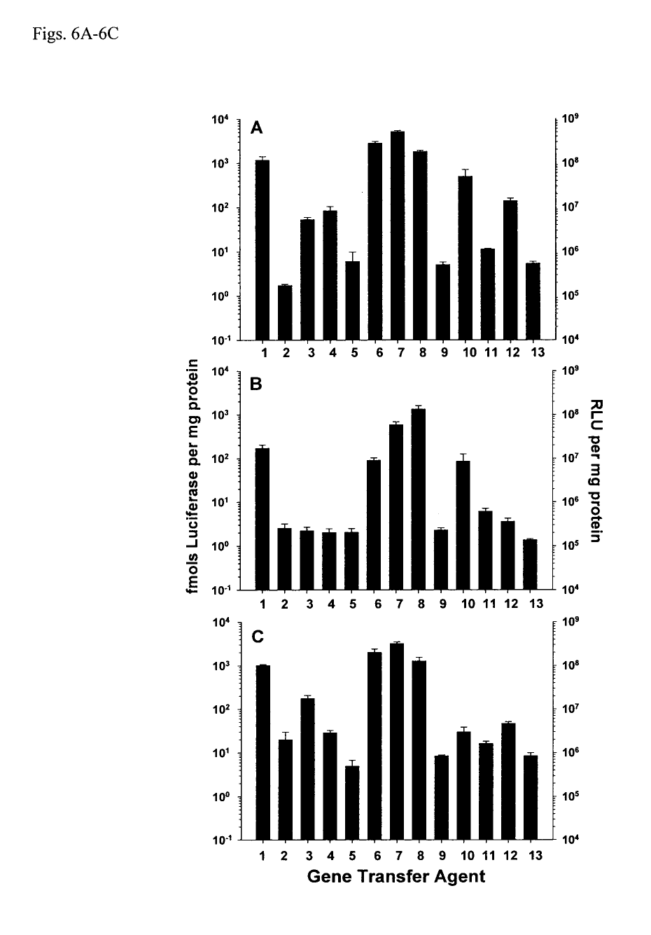

A broader panel of polyacridine-melittin peptides were combined with pGL3 at a fixed stoichiometry of 0.5 nmol per μg of pGL3 and used to mediate gene expression in HepG2, 3T3, and CHO cells. As illustrated in FIGS. 6A-6C, polyacridine-melittin peptides of the poly(Acr-Arg)₂₋₄-SS-melittin (SEQ ID NOS 76-78, respectively) series produced the highest gene transfection in all three cell lines, with levels similar to that of PEI (9:1 N:P). By comparison, poly(Acr-Lys)₄-SS-melittin (SEQ ID NO: 80) and poly(Acr-Leu)₄-SS-melittin (SEQ ID NO: 81) were approximately 10-100 fold less active in gene transfer that poly(Acr-Arg)₄-SS-melittin (SEQ ID NO: 78) in all three cell lines. Reducible poly(Acr-Arg)₂₋₄-SS-melittin peptides (SEQ ID NOS 76-78, respectively) were 100-1000 fold more active in gene transfer compared to poly(Acr-Arg)₄-Mal-melittin (SEQ ID NOS 11 & 67, respectively) in all three cell lines. The importance of the reductive release of melittin was also determined for poly(Acr-Lys)₄-SS-melittin (SEQ ID NO: 80) and poly(Acr-Leu)₄-SS-melittin (SEQ ID NO: 81) in all three cell lines.

To establish the contribution of poly-acridine to DNA binding affinity and gene transfer, the ε-amine of lysine was modified with an acetyl group to prepare poly(Lys(Ac)-Arg)₄-SS-melittin (SEQ ID NO: 79). Gene transfer mediated by poly(Lys(Ac)-Arg)₄-SS-melittin (SEQ ID NO: 79) was approximately 100-1000 fold less relative to poly(Acr-Arg)₄-SS-melittin (SEQ ID NO: 78) in all three cell lines (FIGS. 6A-6C). To further establish the importance of the anchor peptides, Cys-melittin (SEQ ID NO: 52) and Dimeric-melittin (Core sequence disclosed as SEQ ID NO: 74) were used to mediate gene transfer (FIGS. 6A-6C). Both mediated gene transfer that was 10-100 fold lower than poly(Acr-Arg)₂₋₄-SS-melittin (SEQ ID NOS 76-78, respectively), demonstrating the importance of the polyacridine anchor in this gene transfer system.

The toxicity of polyacridine DNA polyplexes was assessed by MTT assay on CHO, HepG2 and 3T3 cells (Table 2). Poly(Acr-Arg)₂₋₃-SS-melittin (SEQ ID NOS 76 & 77, respectively) DNA polyplexes display no toxicity relative to control. However, poly(Acr-Arg)₄-SS-melittin (SEQ ID NO: 78) and poly(Acr-Arg)₄-Mal-melittin polyplexes (SEQ ID NOS 11 & 67, respectively) resulted in 70% cell viability while poly(Lys(Ac)-Arg)₄-SS-melittin polyplex (SEQ ID NO: 79) was more toxic producing 55% viability. CHO cells treated with poly(Acr-Lys)₄-SS-melittin (SEQ ID NO: 80) and poly(Acr-Lys)₄-Mal-melittin polyplexes (SEQ ID NOS 71 & 67, respectively) were 75-86% viable, respectively. Whereas, poly(Acr-Leu)₄-SS-melittin polyplexes (SEQ ID NO: 81) were compared to poly(Acr-Leu)₄-Mal-melittin polyplexes (SEQ ID NOS 72 & 67, respectively) that were minimally toxic (Table 2).

Discussion

The endosome has been proposed as one of the major barriers that limits the gene transfer efficiency of non-viral gene delivery systems. To increase the endosomal escape of plasmid DNA, many prior studies have incorporated fusogenic peptides into experimental delivery systems. Most often, a fusogenic peptide is linked to a PEI or polylysine to allow for multivalent reversible ionic binding with the phosphate backbone of DNA. While this approach often leads to significant increases in gene transfer efficiency in vitro, ionic interactions are relatively weak leading to premature dissociation of the carrier in vivo and degradation of the DNA. To overcome weak binding, higher molecular weight polycations are used. These can be prepared with reducible linkages that revert to low molecular weight, lower affinity polycations and trigger the release of DNA inside the cell. However, despite numerous attempts, i.v. dosed polycationic DNA polyplexes are unable to produce therapeutic levels of gene expression.

We have therefore explored an alternative method of binding fusogenic peptides to plasmid DNA by reversible intercalation. Preliminary studies suggested that a single acridine binds to DNA through intercalation but with relatively weak affinity. Consequently, we sought to increase the binding affinity by preparing polyacridines. Considering the pioneering studies of Szoka who developed a synthetic scheme to prepare a diacridine glycopeptide (Haensler, J., and Szoka, J. F. C. (1993) Synthesis and characterization of a trigalactosylated bisacridine compound to target DNA to hepatocytes. Bioconjugate Chemistry 4, 85-93), and Nielsen who prepared and tested di and triacridine nuclear localization peptides (Shiraishi, T., Hamzavi, R., and Nielsen, P. E. (2005) Targeted Delivery of Plasmid DNA into the Nucleus of Cells via Nuclear Localization Signal Peptide Conjugated to DNA Intercalating Bis- and Trisacridines Bioconjugate Chemistry 16, 1112-1116), we sought a simplified approach that would allow us to extend the valency of polyacridine to four or more acridine units and control the charge of polyacridine DNA polyplexes. We therefore adopted a strategy reported by Ueyama et al. (Ueyama, H., Takagi, M., Waki, M., and Takenaka, S. (2001) DNA binding behavior of peptides carrying acridinyl units: First example of effective poly-intercalation. Nucleic Acids Symp Ser (Oxf) 1, 163-164), who demonstrated that polyacridine peptides could be prepared from Fmoc-Lys(Acr) using solid phase synthesis. Several early attempts at synthesis established that polyacridine peptides could be prepared in good yield (>30%) provided that coupling was conducted with HBTU and that a spacing amino acid was used that had a bulky side chain or protecting group. Using Gly as a spacing amino acid resulted in poor yields.

The synthetic design of polyacridine peptides took into account the relative DNA binding affinity by varying polyacridine repeat, the influence of the spacing amino acid on gene transfer, and linkage between polyacridine and a melittin fusogenic peptide. As indicated in FIGS. 5 and 6A-6C, there is a strong dependency on the nature of the linkage between polyacridine and melittin in relationship to the gene transfer efficiency. A reducible disulfide bond provided a means for the triggered release of melittin either at or inside the cell. The reduction of the disulfide bond greatly diminishes the affinity of melittin for DNA, releasing it and unmasking its membrane lytic activity (FIG. 2C). The released melittin apparently enhances gene transfer by lysis of endosomal membranes that then allows the DNA to more efficiently reach the cytosol and the nucleus, resulting in gene expression that equals or surpasses that of PEI in multiple cell lines (FIGS. 6A-6C). In support of this mechanism, polyacridine linked to melittin by a non-reducible maleimide is 1000-fold less active in gene transfer, despite its equivalent membrane lysis potency against RBCs (Table 1).

The length of the polyacridine repeat appears to be less important for in vitro gene transfer, since repeats of 2, 3 and 4 poly(Acr-Arg)-SS-melittin (SEQ ID NOS 76-78, respectively) all produced nearly equivalent gene transfer in three cell lines (FIGS. 6A-6C). However, it is clear that poly(Acr-Arg)₂-SS-melittin (SEQ ID NO: 76) has lower affinity for DNA as indicated by its ability to displace an intercalator dye (FIG. 2B). The lower affinity results in larger DNA polyplexes as determined by QELS (Table 2) and AFM (FIG. 3B). However, larger polyplexes also sediment more efficiently, facilitating in vitro gene transfer.

The spacing amino acid of Arg, Lys or Leu within a poly(Acr-X)₄-SS-melittin (SEQ ID NOS 78, 80, and 81, respectively) only slightly influences their affinity for binding DNA (FIG. 2C). Each of the resulting polyplexes possessed comparable size, charge and shape as determined by QELS, zeta potential and AFM (Table 2, FIG. 3). However, the gene transfer efficiency was influenced significantly by the identity of the spacing amino acid in the order of Arg>Lys>Leu (FIGS. 6A-6C). In each case, and in each cell line, the reducible polyacridine-melittin peptide mediated greater expression than the non-reducible analogue. The mechanism by which the spacing amino acid influences gene transfer efficiency is unclear. However, it is clear that polyacridine is necessary to mediate significant gene transfer. A control peptide, poly(Lys(Ac)-Arg)₄-SS-melittin (SEQ ID NO: 79), was fully active in membrane lysis but proved to be approximately 1000-fold less active than poly(Acr-Arg)₄-melittin (“(Acr-Arg)4” disclosed as SEQ ID NO: 26) in mediating gene transfer (FIGS. 6A-6C).

The lytic potency of melittin on cells in culture has been well documented. However, this toxicity can be overcome by polymerization of melittin through disulfide bonds resulting in its neutralization due to high affinity binding to DNA. It is apparent that even dimeric-melittin (Core sequence disclosed as SEQ ID NO: 74) can bind to DNA and mediate moderate gene transfer (FIGS. 6A-6C). The MTT assay did establish that polyacridine-melittin peptides are moderately toxic (approx 50-70% viable) when added to DNA at 0.5 nmols of peptide per μg of DNA or higher (Table 2). However, this toxicity is the result of saturation of the DNA with polyacridine peptide resulting in unbound peptide that can lyse cells.

In conclusion, we have described a new polyacridine DNA binding anchor that, when linked with melittin through disulfide bond, produces potent in vitro gene transfer. The polyacridine anchor is designed to be optimized for either in vitro or in vivo gene transfer by controlling the binding affinity to DNA and charge of resulting DNA polyplexes. Furthermore, the Cys residue allows coupling of fusogenic peptides, ligands and PEG. These attributes should allow polyacridine several unique advantages compared to other polycationic DNA binding peptides and polymers.

### Example 2

### Discovery of Metabolically Stabilized Electronegative Polyacridine-PEG Peptide DNA Open Polyplexes

Cationic condensing peptides and polymers bind electrostatically to DNA to form cationic polyplexes. While many cationic polyplexes are able to achieve in vitro transfection mediated through electrostatic interactions, few have been able to mediate gene transfer in vivo. The present study describes the development and testing of polyacridine PEG-peptides that bind to plasmid DNA by intercalation resulting in electronegative open polyplex DNA. (Fernandez, C. A, Baumhover, N. J., Anderson, K., and Rice, K. G. “Discovery of Metabolically Stabilized Electronegative Polyacridine-PEG Peptide DNA Open Polyplexes” Bioconjugate Chemistry (2010) 21, 723-30.) Polyacridine PEG-peptides were prepared by chemically conjugating 6-(9-acridinylamino) hexanoic acid onto side chains of Lys in PEG-Cys-Trp-(Lys)_{3, 4, or 5} (SEQ ID NOS 17-19, respectively). The resulting PEG-Cys-Trp-(Lys-(Acr))_{3, 4, or 5} peptides (SEQ ID NOS 3-5, respectively) bound tightly to DNA by polyintercalation, rather than electrostatic binding. Unlike polycationic polyplexes, polyacridine PEG-peptide polyplexes were anionic and open coiled, as revealed by zeta potential and atomic force microscopy. PEG-Cys-Trp-(Lys-(Acr))₅ (SEQ ID NO: 5) showed the highest DNA binding affinity and the greatest ability to protect DNA from metabolism by DNase. Polyacridine PEG-peptide DNA open polyplexes were dosed intramuscularly and electroporated in mice to demonstrate their functional activity in gene transfer. These results establish polyacridine PEG-peptide DNA open polyplexes as a novel gene delivery method for in vivo use.

Nonviral gene delivery systems have traditionally relied on reversible binding between cationic carriers and anionic oligonucleotides. The resulting lipoplexes and polyplexes typically possess a positive surface charge as determined by zeta potential measurements. While cationic DNA lipoplexes and polyplexes have shown the ability to mediate in vitro gene transfer through electrostatic binding to cell surfaces, they exhibit poor gene transfer properties in vivo, in part due to their overall cationic charge. There have been many attempts to mask the positive charge of cationic polyplexes with polyethylene glycol (PEG) to improve blood compatibility.

Alternatively, it is difficult to prepare an electronegative DNA polyplex using cationic polymers. This has been attempted by either partially titrating a polycationic polymer with plasmid DNA or by using a layer-by-layer addition of oppositely charge polymers in an attempt to reverse the charge of polyplexes. The resulting electronegative polyplexes are formed in a delicate and unstable equilibrium. Attempting to reverse the charge of a cationic polyplex with the addition of an anionic polymer can lead to dissociation of the DNA. Similarly, if titrated to below the charge equivalency point, the polyplex is not completely protected against DNase, whereas if titrated to charge equivalency, the resulting neutral polyplexes are hydrophobic and aggregate. Titration past the equivalency point, results in collapse of a polyplex into a cationic colloidal particle that is relatively stable but highly cationic and thereby less compatible with blood components. Consequently, we sought to find an alternative approach to ionic binding to DNA that would result in stable electronegative polyplexes.

In principle, the high affinity DNA binding achieved by polyintercalation could result in metabolically stable electronegative polyplexes. Electronegative polyplexes may bind to fewer proteins and thereby be more blood and tissue compatible to allow delivery via intramuscle electroporation (IM-EP).

Polyacridine containing polymers have been previously investigated as gene transfer agents by Szoka, Vierling and Neilsen. Their studies utilized polyacridine polymers possessing either one, two or three acridine units conjugated to either a neoglycopeptide or a nuclear localizing sequence. While these polyacridine carriers could bind to DNA, they possessed modest gene transfer activity in vitro. Alternatively, we have recently reported that a polyacridine peptide modified with a melittin fusogenic peptide is a potent in vitro gene transfer agent as described in Example 1 above. The most potent polyacridine-melittin gene delivery peptide contained four acridines and possessed a sequence of Lys(Acr)-Arg-Lys(Acr)-Arg-Lys(Acr)-Arg-Lys(Acr)-Arg-Cys (SEQ ID NO: 87) joined to melittin by a disulfide bond. Polyacridine-melittin bioconjugates formed electropositive DNA polyplexes in which their potency was dependent upon the number of acridines, the identity of the spacing amino acid, and the presence of a reducible disulfide bond between polyacridine and melittin.

To date, there are no reports of polyacridine peptides modified with PEG. The present study demonstrates a synthetic strategy to modify the side chains of Lys in PEG-Cys-Trp-(Lys)ₙ peptides with acridine. The resulting PEG-Cys-Trp-(Lys(Acr))ₙ peptides bind reversibly to DNA through intercalation and demonstrate the ability to form a unique electronegative open polyplex DNA structure that is protected from DNase and maintains transfection competency in vivo. The unique properties of PEGylated polyacridine DNA electronegative open polyplexes afford desirable characteristics that are more compatible with in vivo nonviral gene delivery compared to cationic polyplexes.

Materials and Methods

N-terminal Fmoc protected amino acids, 9-hydroxybenzotriazole (HOBt), diisopropylcarbodiimide (DIC), and Wang resin were purchased from Advanced ChemTech (Lexington, Ky.). Sephadex G-25, HEPES buffer, tris(2-carboxyethyl)-phosphine hydrochloride (TCEP), diisopropylethylamine (DIPEA), piperidine, acetic anhydride, triisopropylsilane (TIS), DNase I (EC 3.1.21.1), 9-chloroacridine, and thiazole orange were obtained from Sigma Chemical Co (St. Louis, Mo.). Acetonitrile, N,N-Dimethylformamide (DMF), and trifluoroacetic acid (TFA) were purchased from Fisher Scientific (Pittsburgh, Pa.). Agarose was obtained from Gibco-BRL. mPEG-maleimide 5000 Da was purchased from Nektar (Huntsville, Ala.), mPEG-amine (5,000 Da) was purchased from Creative Biotechnology (Winston-Salem, N.C.), and bis-amino PEG (2,000 Da) was purchased from Iris Biotech GmbH (Marktredwitz, Germany). The 5.3 kb luciferase plasmid, pGL3 control vector, containing a SV40 promoter and enhancer was obtained from Promega (Madison, Wis.). Plasmid DNA was amplified in DH5α strain of E. coli and purified according to the manufacturer's instructions.

Synthesis and Characterization of PEGylated Cys-Trp-Lysₑ Peptides

Cys-Trp-Lysₙ synthesis was carried out using standard Fmoc chemistry on a computer interfaced Advanced Chemtech APEX 396 solid phase peptide synthesizer with 9-hydroxybenzotriazole and diisopropylcarbodiimide double couplings followed by N-capping with acetic anhydride as described previously(Chen, C. P., Park, Y., and Rice, K. G. (2004) An improved large-scale synthesis of PEG-peptides for gene delivery. Journal of Peptide Research 64, 237-43). Cleavage and deprotection were accomplished by reacting the resin with TFA:TIS:water (95:2.5:2.5) at RT for 3 hrs. The crude peptide was precipitated in cold diethyl ether, centrifuged, and the supernatant was decanted. Crude peptides were reconstituted in 0.1% TFA and purified on a preparatory RP-HPLC using a Vydac C18 column (2×25 cm) eluted at 10 ml/min with 0.1 v/v % TFA and a gradient of acetonitrile of 5-25% over 30 min while monitoring Abs 280 nm. Purified peptides were reconstituted in 0.1% TFA and quantified by Abs (Trp ε₂₈₀ₙₘ=5600 M⁻¹ cm⁻¹) to determine the isolated yield of 30%. The purified peptides were characterized using LC-MS by separation on a Vydac C18 analytical column (0.47×25 cm) eluted at 0.7 ml/min with 0.1% TFA and a gradient of acetonitrile of 5-25% over 30 min while detecting ESI-MS in the positive ion mode.

PEGylation of the Cys residue on Cys-Trp-Lys_{3, 4 and 5} (SEQ ID NOS 14-16, respectively) was achieved by reacting 0.9 mol equivalents of peptide with 1 mol equivalent of PEG_{5000 Da}-maleimide in 100 mM sodium phosphate buffer pH 7 for 1 hr at RT. PEGylated peptides were purified on a G-25 column (2.5×50 cm) eluted with 0.1% acetic acid while monitoring Abs 280 nm. The peak corresponding to the PEG-peptide eluted in the void volume (100 ml), was pooled, concentrated by rotary evaporation, and freeze-dried. The PEG-peptide was reconstituted in 0.1% TFA, and quantified by Abs (Trp ε₂₈₀ₙₘ=5600 M⁻¹ cm⁻¹) to determine an isolated yield of 80%. PEG-peptides were characterized by MALDI-TOF MS by combining 1 nmol with a-cyano-4-hydroxycinnamic acid (CHCA) prepared in 50% (v/v) acetonitrile and 0.1% TFA. Samples were spotted onto the target and ionized on a Bruker Biflex III Mass Spectrometer operated in the positive ion mode.

Synthesis of Polyacridine PEG Peptides and Mono and Bis-Acridine PEGs

6-(9-Acridinylamino) hexanoic acid was prepared according to a prior published procedure (Karup, G., Meldal, M., Nielsen, P. E., and Buchardt, 0. (1988) 9-Acridinylpeptides and 9-acridinyl-4-nitrophenylsulfonylpeptides. Synthesis, binding to DNA, and photoinduced DNA cleavage. Int J Pept Protein Res. 32, 331-343). The carboxyl group on 6-(9-acridinylamino) hexanoic acid was activated with 1.1 mol equivalents of DIC and HOBt in DMF. Activated 6-(9-acridinylamino) hexanoic acid (12 mol equivalents) was reacted with 2.5 μmmol of PEG-Cys-Trp-Lys_{3, 4 and 5} (SEQ ID NOS 17-19, respectively) in 3 ml of DMF at RT for 24 hrs. The PEG-Cys-Trp-(Lys(Acr))_{3, 4 and 5} peptides (SEQ ID NOS 3-5, respectively) were purified using a G-25 column (2.5×50 cm) eluted with 0.1% acetic acid while monitoring Abs 280 nm. The product peak eluting at 100 ml was pooled, freeze dried, and characterized using MALDI-TOF MS as described above. A yield of 42% of PEG-Cys-Trp-(Lys(Acr))_{3, 4, and 5} peptides (SEQ ID NOS 3-5, respectively) was determined by acridine Abs (ε₄₀₉ₙₘ=9266 M⁻¹ cm⁻¹) assuming additivity of acridine molar absorptivity and complete conjugation to the ε-amine of Lys. Similarly, mono and bis-acridinylated PEGs were prepared by reacting 6 mol equivalents of activated 6-(9-acridinylamino) hexanoic acid with PEG-amine or bis-amino PEG (2 μmol) in 2 ml of DMF. The acridinylated PEGs were purified by G-25 column and characterized by MALDI-TOF MS.

Thiazole Orange Displacement Assay

The binding of polyacridine PEG-peptides to DNA was determined using an intercalator dye displacement assay (McKenzie, D. L., Kwok, K. Y., and Rice, K. G. (2000) A potent new class of reductively activated peptide gene delivery agents. Journal of Biological Chemistry 275, 9970-9977). pGL3 (1 μg) was prepared in 5 mM Hepes pH 7.4 containing 40 nM thiazole orange. Polyacridine PEG-peptides were added to pGL3 to prepare samples of 0, 0.25, 0.5, 1, 2, 3, and 4 nmol per μg of DNA in a final volume of 500 μL. The fluorescence intensity of each sample was measured using an LS50B fluorometer (Perkin-Elmer, UK) with an excitation wavelength of 500 nm while monitoring emission at 530 nm. The data was converted to percent by comparison to the fluorescence intensity of thiazole orange in buffer (0%) and pGL3 with fully bound thiazole orange (100%).

Gel Band Shift and DNase Protection Assay

pGL3 (1 μg) or DNA polyplexed (1 μg) prepared in 18 μl of normal saline were combined with 1 nmol of either mono-acridine PEG, bis-acridine PEG, PEG-Cys-(Lys-Acr)₃ (SEQ ID NO: 20), PEG-Cys-(Lys-Acr)₄ (SEQ ID NO: 21), or PEG-Cys-(Lys-Acr)₅ (SEQ ID NO: 22) and 2 μl of loading buffer (Adami, R. C., and Rice, K. G. (1999) Metabolic stability of glutaraldehyde cross-linked peptide DNA condensates. Journal of Pharmaceutical Sciences 88, 739-746). The samples were loaded onto a 1% agarose gel (50 ml) and electrophoresed in TAE buffer at 80 V for 90 min. The gel was stained in 2 mg/ml ethidium bromide at 5° C. overnight, and the transilluminated gels were photographed using Polaroid 667 film. pGL3 (1 μg) or DNA polyplexes (1 μg) prepared with 1 nmol of either mono-acridine PEG, bis-acridine PEG, PEG-Cys-(Lys-Acr)₃ (SEQ ID NO: 20), PEG-Cys-(Lys-Acr)₄ (SEQ ID NO: 21), or PEG-Cys-(Lys-Acr)₅ (SEQ ID NO: 22) in 20 μl of normal saline was incubated with 0.06 U of DNase I at 37° C. for 0-20 min. The samples were applied to a 1% agarose gel and electrophoresed as described above.

Particle Size and Zeta Potential of Polyacridine PEG-peptide DNA Open Polyplexes

Polyacridine PEG-peptide DNA polyplexes were formed by combining 10 μg of pGL3 (in 500 μl of 5 mM Hepes pH 7.4) with an equal volume containing either 1, 2, 4, 8, 12, 16, or 20 nmol of polyacridine PEG-peptide while vortexing. The polyplexes were equilibrated at RT for 30 min prior to analysis of particle size (QELS) and zeta potential using a Brookhaven ZetaPlus (Brookhaven Instruments, Holtsville, N.Y.). The particle size diameter and zeta potential are reported as the mean and standard deviation of ten measurements.

Atomic Force Microscopic Analysis of Polyacridine PEG-peptide DNA Open Polyplexes

pGL3 was prepared at a concentration of 100 μg per ml in 10 mM Tris containing 1 mM EDTA pH 8. DNA polyplexes were prepared identically with the addition of PEG-Cys-Trp-(Lys(Acr))₅ (SEQ ID NO: 5) at either 0.2 or 1 nmol per μg of DNA. pGL3 and DNA polyplexes were diluted 10-fold in 40 mM Hepes containing 5 mM nickel chloride pH 6.7, then deposited on a fresh cleaved mica surface and incubated for 10 min, followed by washing with deionized water. Alternatively, polyacridine-melittin DNA polyplexes were prepared at 0.5 nmol of peptide per μg of DNA at a concentration of 100 μg per ml of DNA and directly deposited on a freshly cleaved mica surface and allowed to bind for 10 min prior to washing with deionized water. Images were captured using an Asylum atomic force microscope (AFM) MFP3D (Santa Barbara, Calif.) operated in the AC-mode using a silicon cantilever (Ultrasharp NSC15/AIBS, Mikro Masch).

Intramuscle-Electroporation Dosing

Male ICR mice (Harlan), weighing 25-30 g were prepared for IM-EP dosing by anesthetization with an intraperitoneal dose of 200 μl of ketamine/xylazine (20 mg/ml and 2 mg/ml, respectively). The hamstring muscles were sheared and swabbed with 70% ethanol prior to administering a 50 μl dose over 10 sec in normal saline to both gastrocnemius muscles in 2 mice by a 1 ml monoject syringe (1 cc, 28 G×½). After dosing the BTX 2-Needle Array Electrode was inserted into the skin with the electrodes straddling the dosing site. Successive electronic stimulation was generated by the EMC BTX 830, Square-wave Pulse Generator (BTX, Harvard Apparatus) as the power source. At 1 min post-administration of the DNA dose, the pulse generator delivered six successive 100 V pulses over 20 msec with a 100 msec interval between pulses (31).

Bioluminescence Imaging

Luciferase reporter gene expression was quantified by bioluminescence imaging (BLI) at 2-14 days following IM-EP using an IVIS Imaging System 200 Series (Xenogen) (Wu, J. C., Sundaresan, G., Iyer, M., and Gambhir, S. S. (2001) Noninvasive optical imaging of firefly luciferase reporter gene expression in skeletal muscles of living mice. Molecular Therapy: the Journal of the American Society of Gene Therapy 4, 297-306). Mice were anesthetized by isofluorane (2% flow with oxygen) and dosed intramuscularly with 40 μL of 30 mg per ml D-Luciferin (GoldBio). Bioluminescent images were acquired 5 min post substrate administration and acquired for 1 min with a 24.6 cm field of view. The resultant grayscale images with a colormap overlay were analyzed using the Igor Pro 4.09 software (Livinglmage). Luciferase expression is reported as photons/sec/cm²/steradian within a uniformly defined region of interest.

Results

Several polyintercalator constructs that bind to double stranded DNA have been reported in prior studies aimed at delivering plasmid DNA (Haensler, J., and Szoka, J. F. C. (1993) Synthesis and characterization of a trigalactosylated bisacridine compound to target DNA to hepatocytes. Bioconjug. Chem. 4, 85-93; Boulanger, C., Di Giorgio, C., and Vierling, P. (2005) Synthesis of acridine-nuclear localization signal (NLS) conjugates and evaluation of their impact on lipoplex and polyplex-based transfection. Eur J Med Chem 40, 1295-306; Shiraishi, T., Hamzavi, R., and Nielsen, P. E. (2005) Targeted Delivery of Plasmid DNA into the Nucleus of Cells via Nuclear Localization Signal Peptide Conjugated to DNA Intercalating Bis- and Trisacridines Bioconjugate Chemistry 16, 1112-1116). These studies designed and tested delivery molecules possessing one-three acridine dyes displayed on a branched polymer modified with a neoglycopeptide or a nuclear localizing peptide, the later of which were shown to mediate in vitro gene expression. These studies established that two to three acridines was required to achieved sufficient DNA binding affinity for in vitro transfection.

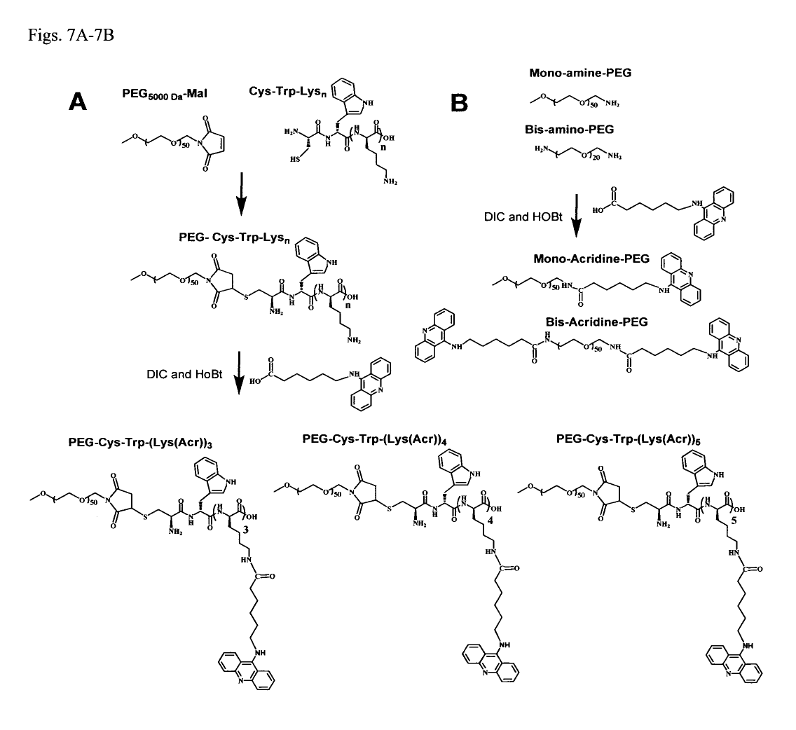

In the present study we sought to use a synthetic strategy that would allow for the incorporation of more than three acridines by covalent modification of the side chain of PEGylated polylysine. This was accomplished by first modifying acridine to possess a 6-amino hexanoic acid and then activating the carboxyl group to allow coupling to the ε-amines of PEG-Cys-Trp-(Lys)_{3, 4 and 5} (SEQ ID NOS 17-19, respectively) to afford PEG-Cys-Trp-(Lys(Acr))_{3, 4 and 5} (SEQ ID NOS 3-5, respectively) (FIGS. 7A-7B). In addition, conjugation of acridine 6-amino hexanoic acid directly to PEG-amine and bis amino PEG afforded constructs with either one or two acridines (FIGS. 7A-7B). This strategy provided an advantage by allowing the comparison of DNA binding affinity with PEGs modified with 1-5 acridines.

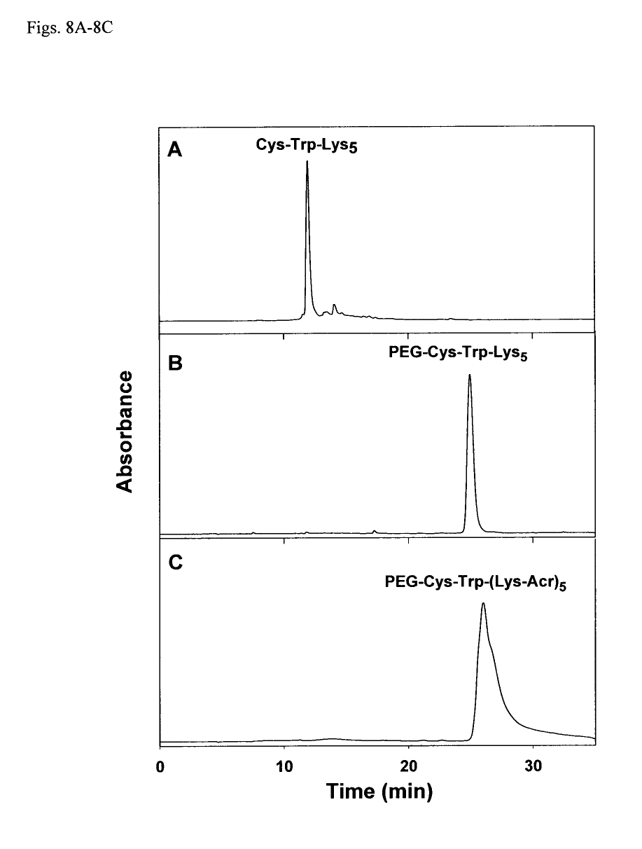

Chromatographic evidence supporting the synthesis of PEG-Cys-Trp-(Lys(Acr))₅ (SEQ ID NO: 5) is illustrated in FIGS. 8A-8C. Modification of Cys-Trp-(Lys)₅ (SEQ ID NO: 16) with PEG results in a significant delay in elution on RP-HPLC compared to Cys-Trp-(Lys)₅ (SEQ ID NO: 16). However, upon conjugation of five acridines to PEG-Cys-Trp-(Lys)₅ (SEQ ID NO: 19), the resulting PEG-Cys-Trp-(Lys(Acr))₅ (SEQ ID NO: 5) elutes only slightly later, and is detected at Abs_{409 nm}.

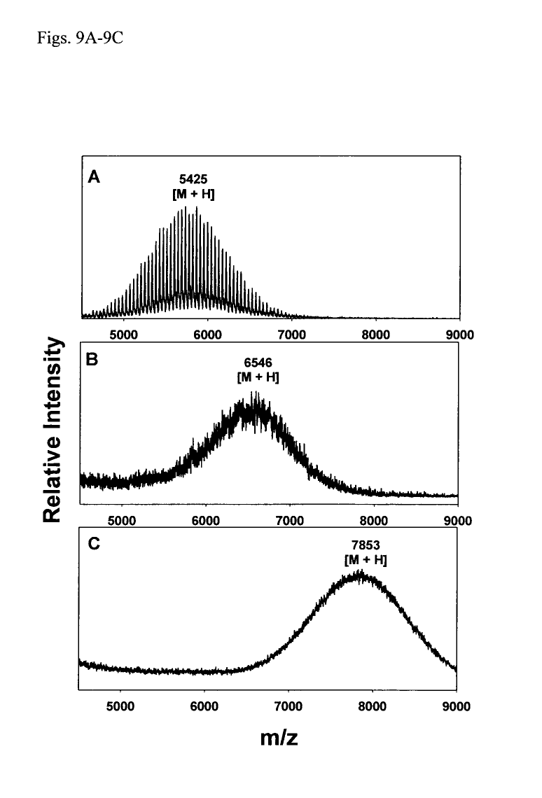

The complete conjugation of acridine to the each Lys side chain was established using MALDI-TOF MS analysis. Conjugation of Cys-Trp-(Lys)₅ (SEQ ID NO: 16) (948.8 g/mol) with polydisperse PEG of average mass of 5425 Da results in a PEG-Cys-Trp-(Lys)₅ (SEQ ID NO: 19) with an apparent average mass of 6546 Da (FIG. 9A-9C). Purified PEG-Cys-Trp-(Lys(Acr))₅ (SEQ ID NO: 5) produced an average mass of 7853 Da (FIG. 3D), consistent with the conjugation of 4.4 acridines. Similar mass spectral results were obtained when preparing PEG-Cys-Trp-(Lys(Acr))₃ (SEQ ID NO: 3) and PEG-Cys-Trp-(Lys(Acr))₄ (SEQ ID NO: 4) as summarized in Table 3.

TABLE 3
MS Analysis of Polyacridine PEG-Peptides
and Monoand Bis-Acridine PEG

| | Mass | Mass |
|---|---|---|
| Sample | (g/mol) | Difference^{b} |
| Mono-amine-PEG | 5385 | 241 (0.8)^{c} |
| Mono-acridine-PEG | 5626 | |
| Bis-amine-PEG | 2000 | 813 (2.8)^{c} |
| Bis-acridine-PEG | 2813 | |
| Cys-Trp-Lys₃^{a} | 691.4 | 714 (2.5)^{c} |
| (SEQ ID NO: 14) | | |
| PEG-Cys-Trp-Lys₃ | 6524 | |
| (SEQ ID NO: 17) | | |
| PEG-Cys-Trp-(Lys(Acr))₃ | 7238 | |
| (SEQ ID NO: 3) | | |
| Cys-Trp-Lys: | 819.5 | 910 (3.1)^{c} |
| (SEQ ID NO: 15) | | |
| PEG-Cys-Trp-Lys₄ | 6623 | |
| (SEQ ID NO: 18) | | |
| PEG-Cys-Trp-(Lys(Acr))₄ | 7533 | |
| (SEQ ID NO: 4) | | |
| Cys-Trp-Lyssa | 948.5 | 1267 (4.4)^{c} |
| (SEQ ID NO: 16) | | |
| PEG-Cys-Trp-Lys₅ | 6555 | |
| (SEQ ID NO: 19) | | |
| PEG-Cys-Trp-(Lys(Acr))₅ | 7822 | |
| (SEQ ID NO: 5) | | |

^{a}Determined by ESI-MS.
^{b}Measured mean difference in MALDI-TOF mass due to the addition of acridine.
^{c}Calculated number of acridine-6-amino-hexanoic acids based on 291 g/mol.

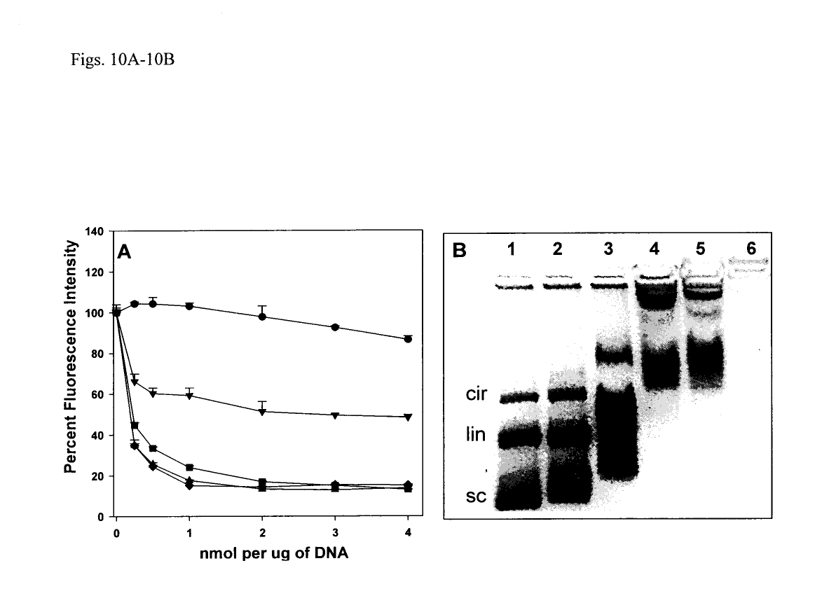

The relative DNA binding affinity of mono and bis-acridine PEG compared to polyacridine PEG-peptides was established using a thiazole orange displacement assay (FIG. 10A). Titration of 0.2-4 nmols of mono-acridine PEG with 1 μg of pGL3 resulted in minimal displacement of thiazole orange. By comparison, bis-acridine PEG demonstrated higher affinity, resulting in a decrease in fluorescence intensity by approximately 40% at 1 nmol per μg of DNA. The apparent DNA binding affinity of PEG-Cys-Trp-(Lys(Acr))₃₋₅ (SEQ ID NOS 3-5, respectively) was higher than either mono or bis-acridine PEG, resulting in approximately 10% fluorescence intensity at a stoichiometry of 1 nmol per μg of DNA (FIG. 10A).

The DNA binding affinity was also compared by a band shift assay performed at a constant stoichiometry of 1 nmol per μg of DNA (FIG. 10B). Mono-acridine PEG failed to produce a band shift relative to plasmid DNA (FIG. 10B lane 1 and 2), whereas bis-acridine PEG partially retarded the migration of DNA (FIG. 10B lane 3). PEG-Cys-Trp-(Lys(Acr))_{3 and 4} (SEQ ID NOS 3 and 4, respectively) both further retarded DNA migration to a similar extent (lanes 4 and 5), whereas PEG-Cys-Trp-(Lys(Acr))₅ (SEQ ID NO: 5) led to complete retardation of DNA migration and also inhibited intense ethidium staining (FIG. 10B. Lane 6). Conversely, PEG-Cys-Trp-(Lys)₅ (SEQ ID NO: 19) was unable to cause a band DNA shift (not shown).

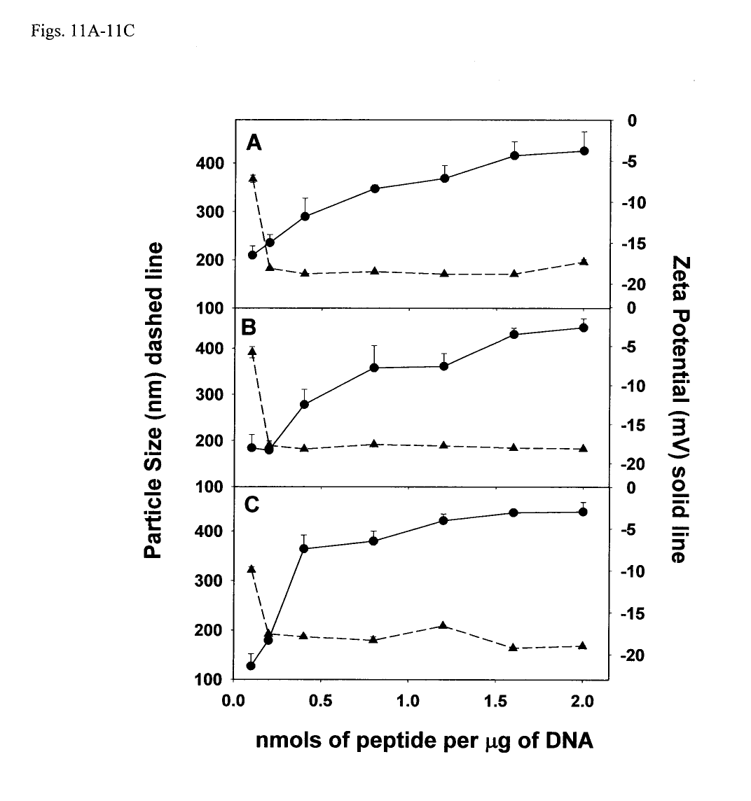

A further demonstration of polyacridine binding to DNA binding utilized a titration experiment while monitoring both the particle size and zeta potential of DNA polyplexes as a function of increasing concentration of polyacridine (FIGS. 11A-11C). The titration of PEG-Cys-Trp-(Lys(Acr))₃ (SEQ ID NO: 3) with pGL3 results in a decrease in the apparent particle diameter to approximately 200 nm at 0.2 nmols per μg of DNA but then plateaued at higher stoichiometries. The zeta potential gradually increased from −15 to −5 mV when titrating with 0.1 to 2 nmol of PEG-Cys-Trp-(Lys(Acr))₃ (SEQ ID NO: 3) per μg of DNA, indicating that the polyplexes remained electronegative (FIG. 11A). A nearly identical trend was observed when titrating pGL3 with PEG-Cys-Trp-(Lys(Acr))₄ (SEQ ID. NO: 4) and PEG-Cys-Trp-(Lys(Acr))₅ (SEQ ID NO: 5) (FIGS. 11B and 11C), with the exception that the latter reached an asymptote of −5 mV at 1 nmol per μg of DNA due to its greater DNA binding affinity.

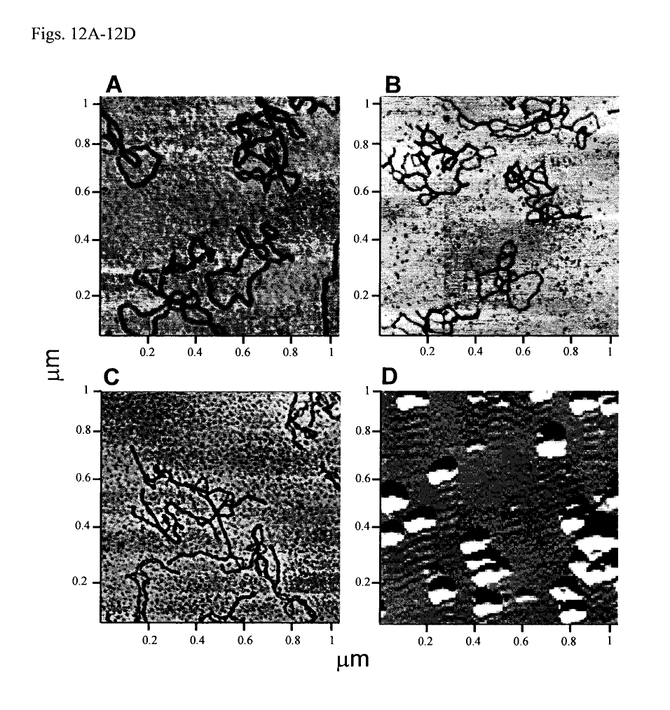

The unusual zeta potential properties of the polyacridine DNA polyplexes prompted an investigation into their morphology using atomic force microscopy. Under analysis by AFM, plasmid DNA is observed as an open circular structure when immobilized onto electropositive mica prepared with immobilized Ni (FIG. 12A). By comparison, PEG-Cys-Trp-(Lys(Acr))₅ DNA polyplexes (SEQ ID NO: 5) prepared at either 0.2 or 1 nmol per μg of DNA also resulted in a open circular structure, which we term open polyplexes (FIGS. 12B and 12C). Comparison of the size and shape of open polyplexes with plasmid DNA reveals a slightly more compact structure that becomes progressively more tightly wound at higher stoichiometry of polyacridine. The appearance of smaller particles in FIGS. 12B-12C are the result of AFM tip irregularities. Finer analysis of the data in FIGS. 6B and C suggests that the thickness of the open polyplex DNA is reduced by 20% relative to naked DNA. The open polyplex structure observed by AFM is quite unique when compared to an electropositive polyplex prepared with polyacridine melittin (FIG. 12D).

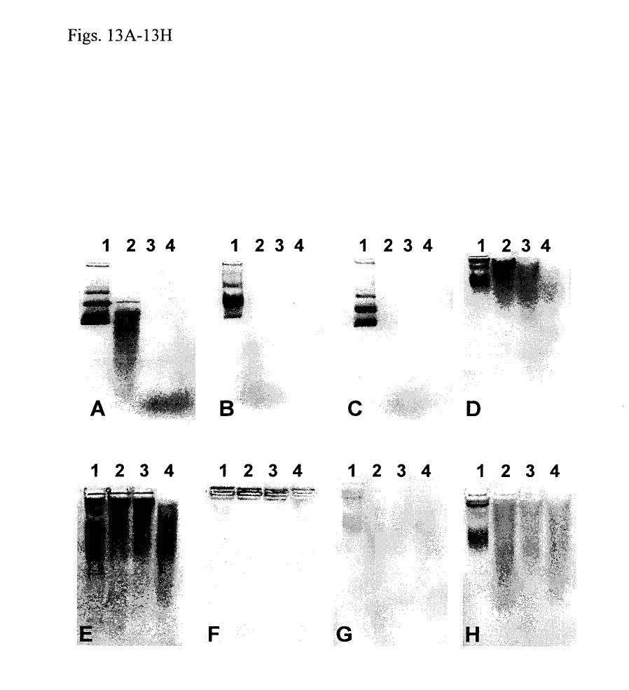

We conjectured that the DNA in open polyplexes may be protected from metabolic degradation by serum endonucleases. The metabolic stability was evaluated by treating pGL3 with DNase followed by gel electrophoresis. Unprotected pGL3 is significantly degraded by DNase in 5 min and completely degraded by 10 min (FIG. 13A, lanes 2 and 3). The weak binding affinity afforded by mono or bis-acridine PEG failed to protect DNA from metabolism (FIGS. 13B and 13C). Comparison of the metabolic stability of PEG-Cys-Trp-(Lys(Acr))_{3, 4 and 5} DNA open polyplexes (SEQ ID NOS 3-5, respectively) demonstrated that PEG-Cys-Trp-(Lys(Acr))₃ (SEQ ID NO: 3) and PEG-Cys-Trp-(Lys(Acr))₄ (SEQ ID NO: 4) provided partial protection (FIGS. 13D and 13E), whereas PEG-Cys-Trp-(Lys(Acr))₅ (SEQ ID NO: 5) provided complete protection (FIG. 13F). Decreasing the stoichiometry from 1 nmol of PEG-Cys-Trp-(Lys(Acr))₅ (SEQ ID NO: 5) to 0.4 or 0.2 nmol per μg of DNA established that at lower stoichiometries the DNA was more susceptible to metabolism (FIGS. 13G and 13H).

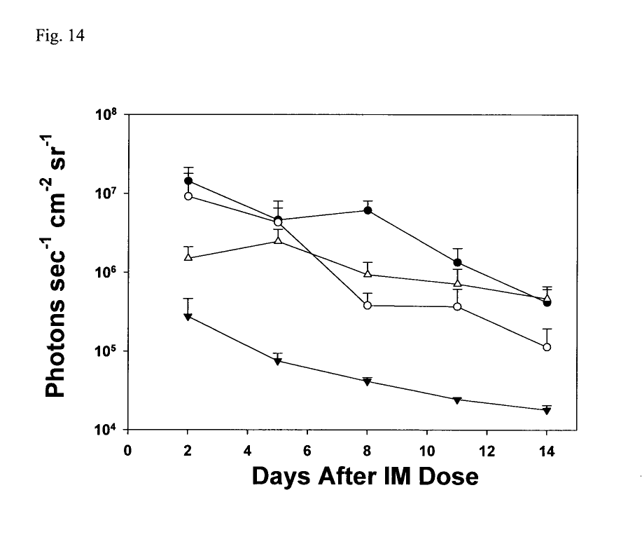

The in vivo gene transfer properties of PEG-Cys-(Lys-(Acr))₅-DNA open polyplexes (SEQ ID NO:22) were evaluated in mice following IM-EP. pGL3 and pGL3 PEG-Cys-(Lys-(Acr))₅ (SEQ ID NO: 22) open polyplexes prepared at 0.2, 2, and 4 nmol per μg of DNA formulations were administered by IM-EP and the expression was monitored for two weeks by bioluminescence imaging (FIG. 14). At day 2 following dosing, pGL3 and the DNA open polyplexes prepared at 2 nmol per μg of DNA produced identical expression levels. Open polyplexes prepared at 4 nmol per μg of DNA showed approximately 10-fold lower expression, whereas a formulation prepared with 0.2 nmol per μg of DNA resulted in a 100-fold decrease in expression. A similar expression time-course was observed for pGL3 and DNA open polyplexes prepared with 0.2 and 2 nmol of PEG-Cys-(Lys-(Acr))₅ (SEQ ID NO: 22) per μg of DNA resulting in a 10-fold decrease over 14 days. The expression time-course for open polyplexes prepared at 4 nmol of PEG-Cys-(Lys-(Acr))₅ (SEQ ID NO: 22) per μg of DNA was extended, resulting in only a 2-3 fold loss in expression across the 14 day sampling period.

Discussion

An important function of a gene delivery carrier is to protect DNA from premature metabolism during transit. The present study demonstrates that polyintercalation provides strong and reversible binding to DNA that delays metabolic degradation. The main advantage of this approach over polycationic polymer binding to DNA is that high affinity can be achieved with a short polyacridine and that the charge of the resulting DNA polyplexes can be controlled to be either positive or negative, depending on intended applications in vitro or in vivo. Prior reports of the cellular toxicity of known intercalators demonstrate a dependency on both chemical structure and dose (Ferguson, L. R., and Denny, W. A. (2007) Genotoxicity of non-covalent interactions: DNA intercalators. Mutat Res 623, 14-23). Little or no information is available regarding the toxicity of PEGylated polyacridine peptides.

To evaluate the relationship between DNA binding affinity and metabolic stability, five DNA carriers were prepared that varied the number of acridines from 1 to 5. The synthetic strategy conjugated 6-(9-acridinylamino) hexanoic acid with the primary amine(s) on PEG-amine, bis-amino PEG, and the c-amines on PEG-Cys-Trp-(Lys)_{3, 4 or 5} (SEQ ID NOS 17-19, respectively). This strategy not only simplified the chemical preparation of the delivery molecules, but also allowed for a long 12 atom tether between each acridine and the peptide backbone to facilitate flexible multivalent binding of acridine with DNA.

The DNA binding affinity significantly increased for PEG-Cys-(Lys-(Acr))_{3, 4,} and ₅ (SEQ ID NOS 20-22, respectively) compared to mono and bis-acridine PEG as determined by both fluorophore displacement and DNA band shift on agarose gel electrophoresis (FIGS. 10A and 10B). While the QELS particle size suggested the formation of large polyplexes with an apparent diameter of 200 nm, the zeta potential revealed that for PEG-Cys-(Lys-(Acr))_{3, 4,} and ₅ polyplexes (SEQ ID NOS 20-22, respectively) were negatively charged. AFM images established that the shape of PEG-Cys-(Lys-(Acr))_{3, 4,} and ₅ DNA polyplexes (SEQ ID NOS 20-22, respectively) were open coiled structures that closely resemble plasmid DNA (FIG. 12). The observed electronegative open polyplexes are distinct from electropositive condensed polyplexes that are formed by polycation binding with DNA. The metabolic stability afforded by comparing PEG-Cys-(Lys-(Acr))_{3, 4 and 5} (SEQ ID NOS 20-22, respectively) establish a relationship between the apparent DNA binding affinity and the degree of protection from DNase. Additionally, the metabolic protection of DNA afforded by PEG-Cys-(Lys-(Acr))₅ (SEQ ID NO: 22) was concentration dependent.

IM-EP of cationic DNA polyplexes results in lower gene expression levels compared to naked DNA alone. This may be due to the charge of cationic polyplexes that are unable to electromigrate through the transient pore formed during electroporation. In contrast, the IM-EP of DNA versus PEG-Cys-(Lys-(Acr))₅ (SEQ ID NO: 22) DNA open polyplexes resulted in very similar levels and duration of expression in mice over two weeks. However, at an elevated stoichiometry of PEG-Cys-(Lys-(Acr))₅ (SEQ ID NO: 22), a lower but more sustained expression was determined, perhaps due to the metabolic protection afforded by the peptide at higher stoichiometries.

The results of this study establish a new family of PEGylated polyacridine gene delivery carriers that bind to DNA and provide protection against DNase. The unique anionic open polyplex structure is compatible with IM-EP. Given the flexibility of design, polyacridine gene delivery systems could be optimized for a variety of other nonviral gene delivery applications.

### Example 3

### Synthesis of High Mannose N-Glycan Bioconjugates as Targeted Gene Delivery Ligands

Glycosylation is crucial to the propagation of pathogenic viral particles in vivo. Glycosylation masks the agent from immune detection and mediates binding and cellular uptake. The Man₉ N-glycan found on the surface of HIV is an example of this type of system. High-mannose N-glycans are found in a variety of plant and animal sources in nature. They bind to a family of calcium dependent receptors present on macrophages and on cells of the immune system. The macrophage mannose receptor binds di-mannose clusters whereas the dendritic cell SIGN receptor recognizes branched high mannose structures.

Similarly, plasmid based DNA vaccines transfect cells more efficiently when a targeting ligand is a component of the vector. To prime the immune system we targeted plasmid DNA into dendritic cells via the mannose binding lectin found on the cell surface. We incorporate a high mannose N-glycan, Man₉GlcNAc₂, in our vector to stimulate a dendritic cell specific response. (Anderson, K, Fernandez, C. A., and Rice, K. G. “N-Glycan Targeted Gene Delivery to the Dendritic Cell SIGN Receptor” Bioconjugate Chemistry (2010) 21, 1479-85.)

Several attempts have been made toward the synthesis of Man₉GlcNAc₂. These syntheses are typically long, multi-step procedures and are low yielding. Our approach involves the purification of soybean agglutinin, which is a galactose specific lectin and glycoprotein containing Man₉GlcNAc₂. A Sepharose N-Caproyl Galactosamine affinity column was constructed and used to isolate SBA in a single chromatographic step from soy flour extract. Affinity purified soybean agglutinin is digested with Pronase to release Man₉GlcNAc₂-Asn, which is then derivatized with tyrosine and a Maleimide derivative of proprionic acid.

A peptide composed of cysteine, lysine, and lysine derivatized with an intercalating dye was synthesized, and conjugated to the maleimide functionality of Man₉GlcNAc₂ (Formula IV). CHO cells with a recombinant DC-SIGN receptor and the PGL3 plasmid are part of our in vitro system to measure the targeting and transfection efficiency of our gene delivery vector.

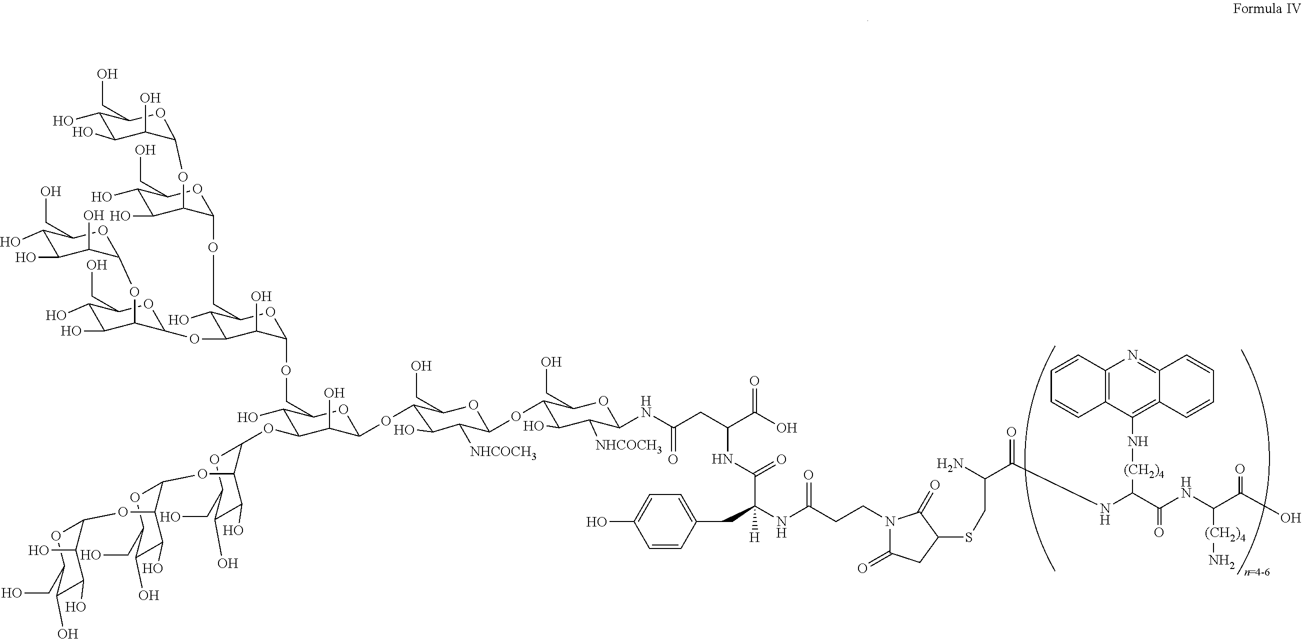

N-Glycan Targeted Gene Delivery to the Dendritic Cell Sign Receptor

A novel non-viral gene delivery vector composed of a high mannose N-glycan conjugated to a polyacridine peptide was prepared. The glycopeptide was designed to bind to plasmid DNA by a combination of polyintercalation and ionic binding, and to the DC-SIGN (Dendritic Cell-Specific Intracellular adhesion molecule-3 Grabbing Non-integrin) receptor expressed on CHO cells by recognition of the high mannose N-glycan. The glycopeptide conjugate was prepared by purification of a high mannose N-glycan from affinity fractionated soybean agglutinin (SBA). The SBA was proteolyzed to release the N-glycan which was then modified on its N-terminus with Tyr and a propionate maleimide. A DNA binding polyacridine peptide, Cys-(Acr-Lys)₄ (SEQ ID NO: 23), was prepared by solid phase peptide synthesis using Fmoc-Lys(Acr), then conjugated to the maleimide on the N-glycan to produce a glycopeptide. The glycopeptide bound to DNA with high affinity as determined by fluorophore displacement assay and DNA band shift on agarose gel. When bound to Cy5 labeled DNA, the glycopeptide mediated specific uptake in DC-SIGN CHO (+) cells as determined by FACS analysis. In vitro gene transfer studies established that the glycopeptide increased the specificity of gene transfer in DC-SIGN CHO (+) cells 100-fold relative to CHO (−) cells. These studies suggest that a high-mannose N-glycan conjugated to a polyacridine peptide may also facilitate receptor mediated gene delivery in dendritic cells and thereby find utility in the delivery of DNA vaccines.

Introduction

Dendritic cells (DC) are the primary antigen presenting cells in the immune system, and are therefore central to the development of vaccines. One of the challenges in developing DNA vaccines is the design of vectors that efficiently and selectively target DCs. The DC-SIGN (Dendritic Cell-Specific Intracellular adhesion molecule-3 Grabbing Non-integrin) receptor is a cell surface endocytosing lectin that is specifically expressed on DCs that binds to glycoprotein N-glycans of the high-mannose or complex type possessing terminal Le^{X} with a μM Kd. The receptor is therefore ideally located to facilitate receptor mediated gene delivery to DCs.

Others have targeted either immunogenic proteins or plasmid DNA to internalize into DCs via DC-SIGN in an attempt to prime the immune system. These studies establish that either encapsulation of DNA into mannosylated liposomes or complexation of DNA with mannosylated-PEI, mannosylated-chitosan, mannan-polylysine, or mannosylated glycolipid, results in gene delivery with improved immune response when administered i.m. or i.p. Other studies have determined that high-mannose N-glycans on remodeled glycoproteins are able to target DC-SIGN resulting in uptake into DCs and enhanced immune response. While gene delivery to DCs with mannose containing polymers and liposomes has been achieved, no study has yet demonstrated the delivery of DNA to DCs using a natural high mannose N-glycan.

Reversible binding of an N-glycan to DNA can be accomplished by covalently linking it to a polylysine peptide resulting in cationic polyplex. However, polylysine peptides of 18 residues or longer (SEQ ID NO: 85) are necessary to achieve sufficient affinity to protect DNA, and the resulting electropositive polyplexes bind non-specifically to numerous proteins in vivo. To overcome these limitations, we recently demonstrated that short cationic peptides possessing multiple acridines attached to the side chain of Lys residues bind DNA with much higher affinity and protect DNA from metabolism compared to much longer polylysine peptides.

Functionalized polyacridine peptides, modified with either PEG or a fusogenic peptide, have been shown to mediate gene transfer in vitro or in vivo. Earlier studies have also demonstrated the efficacy of a polyacridine polymer functionalized with either a nuclear localization signal (NLS) or a neoglycopeptide (Shiraishi, T., Hamzavi, R., and Nielsen, P. E. (2005) Targeted Delivery of Plasmid DNA into the Nucleus of Cells via Nuclear Localization Signal Peptide Conjugated to DNA Intercalating Bis- and Trisacridines Bioconjugate Chemistry 16, 1112-1116; Haensler, J., and Szoka, J. F. C. (1993) Synthesis and characterization of a trigalactosylated bisacridine compound to target DNA to hepatocytes. Bioconjug. Chem. 4, 85-93). These experiments demonstrated the importance of increasing the DNA binding affinity by increasing the number of acridines. An early report by Ueyama also demonstrated that polyacridine peptides possessing two to five Lys(Acr) residues results in progressively higher affinity for calf thymus DNA (Ueyama, H., Takagi, M., Waki, M., and Takenaka, S. (2001) DNA binding behavior of peptides carrying acridinyl units: First example of effective poly-intercalation. NUCLEIC ACIDS SYMP SER (OXF) 1, 163-164).

To date, there have been no reports of the successful development and use of a polyacridine glycopeptide in gene transfer. Here, we report the purification of a high-mannose N-glycan from soybean agglutinin and its conjugation to a polyacridine peptide. The resulting glycopeptide binds to plasmid DNA with high affinity and mediates targeted uptake and gene expression in DC-SIGN expressing cells in vitro.

Materials and Methods

Sepharose CL-4B, D-galactosamine HCl, 6-aminocaproic acid, Boc-tyrosine N-hydroxysuccinimide, 9-chloroacridine (97%), pronase, thiazole orange, phenol, super-DHB diisopropylethylamine, piperidine, triisopropylsilane (TIS), polyethylenimine (PEI), and acetic anhydride were from Sigma-Aldrich (St. Louis, Mo.). Sephadex G-50 resin was from Amersham Biosciences (Pittsburgh, Pa.). AG 50W-X2 cation exchange resin was from Bio-Rad (Hercules, Calif.). 1,1′ carbonyldiimidazole, and N-(3-dimethylaminopropyl)-N′-ethyl-carbodiimide HCl were Fluka Biochemica products (St. Louis, Mo.). 3-maleimidoproprionic acid N-hydroxysuccinimide ester was from ABD Bioquest, Inc (Sunnyvale, Calif.). NuPAGE® Novex Bis-Tris Gels, Dulbecco's Modified Eagle Medium (DMEM), MEM non-essential amino acids, Dulbecco's phosphate buffered saline (DPBS) and fetal bovine serum (FBS) were from Invitrogen (Carlsbad, Calif.). D₂O was from Cambridge Isotope Laboratories (Cambridge, Mass.). Untoasted soy flour was obtained from Archer Daniels Midland (Decatur, Ill.). Sodium hydroxide was a Mallinckrodt product through Fisher Scientific (Pittsburgh, Pa.). Fmoc-Lys-OH was from Novabiochem. Ethidium bromide was from Bio-Rad. Fmoc-Lys(Boc)-Wang resin, Fmoc-Lys(Boc)-OH, Fmoc-Cys(Trt)-OH, N-methyl-2-pyrrolidinone (NMP), O-benzotriazole-N,N,N′,N′-tetramethyl-uronium-hexafluoro-phosphate (HBTU) and hydroxybenzotriazole hydrate (HOBt) were from Advanced ChemTech (Lexington, Ky.). N′,N-dimethylformamide (DMF) was from Amresco (Solon, Ohio). Human DC-SIGN (+) and DC-SIGN (−) CHO cells were a gift from Drs. Chae Gyu Park and Ralph M. Steinman of the Laboratory of Cellular Immunology and Physiology at The Rockefeller University (28). Cy5™ LabelIT was from Mirus Biotechnology (Madison, Wis.). pGL3 control vector, a 5.3 kb luciferase plasmid containing an SV40 promoter and enhancer, was obtained from the Promega corporation (Madison, Wis.), amplified in DH5α E. coli from Invitrogen, and purified with a Giga kit from Qiagen (Valcencia, Calif.). D-Luciferin was from Gold Biotechnology (St. Louis, Mo.), Adenosine 5′-triphosphate was from Roche (Indianapolis, Ind.), and BCA Protein Assay reagents were Pierce protein research products of Thermo Scientific (Rockford, Ill.). All reagents and solvents were certified ACS grade, molecular biology grade, or an equivalent.

Preparation of Sepharose N-Caproylgalactosamine Affinity Resin

6-Aminocaproic acid was coupled to activated Sepharose CL-4B as described (Bethell, G. S., Ayers, J. S., Hancock, W. S., and Hearn, M. T. (1979) A novel method of activation of cross-linked agarose with 1,1′-carbonyldiimidazole which gives a matrix for affinity chromatography devoid of additional charged groups. J Biol Chem 254, 2572-4). Briefly, a moist cake (50 g) of Sepharose CL-4B was activated with 1,1′ carbonyldiimidazole (CDI) (1.2 g). After a 15 min reaction with CDI, the mixture was washed with 1 L of acetone, then reacted with 14 g of 6-aminocaproic acid in 90 ml of water. The pH of the slurry was adjusted to 10 with 1

M sodium hydroxide and stirred overnight at 4° C. Ligand coupling proceeded with the addition of 600 mg of galactosamine and 300 mg of CDI followed by reaction for 2 hrs (Allen, A. K., and Neuberger, A. (1975) A simple method for the preparation of an affinity absorbent for soybean agglutinin using galactosamine and CH-Sepharose. FEBS Letters 50, 362-4). The affinity resin (30 ml) was washed with 90 ml each of 1 M sodium chloride, 1 M sodium chloride in 0.1 M Tris pH 8.6, 1 M sodium chloride in 50 mM sodium formate pH 3.0, 1 M sodium chloride in 0.1 M Tris pH 8.6, water, and PBS.

Purification of Soybean Agglutinin (SBA)

Soy flour (10 g) was dissolved in 200 ml of PBS and stirred overnight at 4° C. The slurry was centrifuged at 9,000×g for 15 min, and the supernatant was applied to the affinity column (2.5×10 cm). The column was washed with 90 ml PBS until the Abs₂₈₀ₙₘ of the effluent was below 0.02 units. The bound SBA was eluted with 90 ml of 0.2 M galactose in PBS (Allen, A. K., and Neuberger, A. (1975) A simple method for the preparation of an affinity absorbent for soybean agglutinin using galactosamine and CH-Sepharose. FEBS Letters 50, 362-4). The SBA was pooled and dialyzed in 12-14 kDa MWCO tubing for 48 hrs against running water and freeze dried.

Purification of Man 9 from SBA

Affinity purified SBA was proteolytically digested to release Man 9 which was then purified following the method of Lis et al. (Lis, H., and Sharon, N. (1978) Soybean Agglutinin—A Plant Glycoprotein. Structure of the Carbohydrate Unit. I Biol. Chem. 253, 3468-3476). Briefly, SBA (600 mg in 8 ml 1 mM hydrochloric acid) was heat denatured at 50° C. for 15 min and digested with 12 mg of pronase for 48 hr at 37° C. The digest was centrifuged at 4,000×g and the supernatant applied to a Sephadex G-50 column (1.5×170 cm) eluted with 10 mM acetic acid. The fractions (8.5 ml) were monitored by Abs 280 nm and an 200 μl aliquot of each fraction was analyzed for carbohydrate by the phenol sulfuric assay (Dubois, M., Gilles, K., Hamilton, J. K., Rebers, P. A., and Smith, F. (1951) A Colorimetric Method for the Determination of Sugars. Nature 168, 167). Carbohydrate containing fractions were pooled, concentrated and re-purified by gel filtration on Sephadex G-50. Man 9 containing fractionations were prepared in 1.5 ml of water then applied to a AG 50W-X2 cation exchange column (1.5×50 cm) eluted with 1.2 L of 1 v/v % pyridine/acetic acid, pH 3.2 while detecting carbohydrate by the phenol-sulfuric assay. The Man 9 containing fractions eluting a 880 ml were pooled and freeze dried. The recovery was estimated by phenol sulfuric assay to be 6.7 μmols (45%) based on 18.5 nmols of Man₉GlcNAc₂Asn=1 AU₄₉₀ₙₘ.

The purified Man 9 was analyzed by mass spectroscopy by preparing 2 nmol in 4 μl of 10 mM sodium chloride containing 0.5 mM of super 2,5-DHB. The sample was spotted on the target and analyzed by MALDI-TOF (Biflex III, Bruker) in reflectron mode resulting in the detection of masses of 2019.72 and 2041.66 m/z corresponding to [M+Na] and [M+2Na].

Man 9 was analyzed by high field NMR (600 MHz Varian) by preparing a 1 mM sample in D₂O with acetone as an internal standard. The chemical shift data corresponded with that published previously (Dorland, L., van Halbeck, H., Vliegenthart, J. F. G., L is, H., and Sharon, N. (1981) Primary Structure of the Carbohydrate Chain of Soybean Agglutinin. A Reinvestigation by High Resolution ¹H NMR Spectroscopy. J. Biol. Chem. 256, 7708-7711).

(SEQ ID NO: 23)
Synthesis of Cys-(Acr-Lys)₄

The amino acid peptide, Cys-(Acr-Lys)₄ (SEQ ID NO: 23), was synthesized using standard Fmoc chemistry with HOBt and HBTU double coupling on a 30 μmol scale with an APEX 396 solid phase peptide synthesizer using Fmoc-Lys(Acr) prepared as described previously (Tung, C., Zhu, T., Lackland, H., and Stein, S. (1992) An acridine amino acid derivative for use in Fmoc peptide synthesis. Peptide Research 5, 115-8). The peptide was cleaved from the resin and deprotected with 2 ml of TFA/TIS/H₂O (95:2.5:2.5 v/v/v) for 3 hr, followed by precipitation in 40 ml of cold ether. The precipitate was collected by centrifugation at 4,000×g for 10 min, and reconstituted in 0.1 v/v % TFA. The peptide was preparatively purified on RP-HPLC by injecting 2 μmols onto a 2×25 cm Vydac C18 column eluted at 10 ml per min with 0.1% TFA and a 15-30% acetonitrile gradient over 45 min while detecting at Abs₂₈₀ₙₘ. The peptide was pooled, concentrated by rotary evaporation, reconstituted in 0.1 v/v % TFA, and then analyzed for purity by RP-HPLC, injecting 5 nmol onto a 0.47×25 cm Vydac C18 column eluted at 1 ml/min with 0.1% TFA and a gradient of 15-30% acetonitrile while detecting by positive mode ESI-MS. The doubly charged ion was detected with an m/z of 928.7, resulting in an observed mass of 1855.4 m/z (calc=1855.5 m/z). The synthesis resulted in a 17% isolated yield (ε_{409 nm}=37, 064 M⁻¹ cm⁻¹) at >95% purity.

Synthesis of a Man 9 Glycopeptide

Man 9 (500 nmol) was prepared in 500 μl of 50 v/v % DMF and 100 mM sodium bicarbonate containing Boc-Tyr-NHS (5 μmol) and reacted for 3 hr at RT. The reaction mixture was directly applied to a Sephadex G-25 column (0.5×50 cm) eluted with 10 mM acetic acid while detecting Abs₂₈₀ₙₘ. The Man 9 Tyr-Boc product peak eluting at 50 ml was concentrated by rotary evaporation and reconstituted in water. The product was analyzed for purity by analytical RP-HPLC eluting at 1 ml per min with 0.1% TFA and a 1-30% gradient of acetonitrile over 30 min while detecting by Abs₂₈₀ₙₘ and by negative mode ESI-MS. The doubly charged ion was detected with an m/z of 1129.4, resulting in a mass of 2260.8 m/z (calculated 2260.0 m/z). The Man 9 Tyr-Boc product was >95% pure and the yield (ε₂₈₀ₙₘ=1130 M⁻¹cm⁻¹) was 95%.

To conjugate Man 9 Tyr-Boc to Cys-(Acr-Lys)₄ (SEQ ID NO: 23), the Boc group was removed from the Tyr and the amine terminus was derivatized with propionate maleimide, prior to reaction with Cys-(Acr-Lys)₄ (SEQ ID NO: 23). The Boc group was removed by treating Man 9 Tyr-Boc (500 nmol) with 50 μl of 95% TFA for 5 min at RT, followed by freeze drying to remove TFA. The yield of Man 9 Tyr was quantitative and LC-MS analysis established a mass of 2160.3 m/z consistent with the anticipated mass (2160.9 m/z). Man 9 Tyr (500 nmol) was reacted for 1 hr with 3-maleimidoproprionic acid NHS ester (5 μmol) in 250 μl of 50 v/v % DMF and 100 mM sodium bicarbonate. The reaction mixture was fractionated on a Sephadex G-25 column (1.5×50 cm), eluting with 0.1% acetic acid while detecting Abs_{280 nm} The product peak (Man 9 Mal), eluting at 35 ml, was pooled and concentrated by rotary evaporation and purified to homogeneity on a semi-prep Vydac C18 RP-HPLC column (2×25 cm) eluted at 10 ml per min with 0.1% acetic acid and a 30 min acetonitrile gradient (5-15%) resulting in a 23% isolated yield. LC-MS analysis in the negative mode revealed that the Man 9 Mal product produced an ion of 1156.1 m/z, resulting in a mass of 2314.2 m/z, closely corresponding to the anticipated mass of 2312.1.

To complete the glycopeptide synthesis, Man 9 Mal (500 nmol) was reacted with Cys-(Acr-Lys)₄ peptide (SEQ ID NO: 23) (600 nmol) in 500 μl of 5 mM Hepes pH 7 for 2 hrs. The Man 9 glycopeptide product was purified from excess dimeric peptide on analytical RP-HPLC with a 30 min 15-30% acetonitrile gradient in 0.1% TFA. The purity and identity of the Man 9 glycopeptide were verified by analytical RP-HPLC detected by Abs₂₈₀ₙₘ and positive mode ESI-MS. The product peak was >95% pure and produced a triply charged ion detected with an m/z of 1390.0 that deconvoluted to an observed mass of 4167.0 m/z (calculated 4167.4 m/z). The recovery was 102 nmol, resulting in a 23% yield.

Man 9 Glycopeptide DNA Band Shift Assay

pGL3 (1 μg) was combined with 0.05, 0.2, 0.5, and 1 nmol of Man 9 glycopeptide. Each sample was incubated for 30 min before loading onto an agarose gel, that was electrophoresed at 80 V for 60 min (Adami, R. C., Collard, W. T., Gupta, S. A., Kwok, K. Y., Bonadio, J., and Rice, K. G. (1998) Stability of Peptide-Condensed Plasmid DNA Formulations. Journal of Pharmaceutical Sciences 87, 678-683). The gel was stained with 0.1 mg per ml of ethidium bromide at 4° C. for 24 hrs, then photographed while illuminating on a transilluminator.

Thiazole Orange Displacement Assay

A control sample of pGL3 (1 μg) was combined with 40 nM thiazole orange in a total volume of 500 μl of 5 mM Hepes buffer pH 7.4. The Man 9 glycopeptide (0.2, 0.3, 0.4, 1 and 2 nmol) or Cys-(Acr-Lys)₄ peptide (SEQ ID NO: 23) was added to pGL3 (1 μg) samples containing thiazole orange, and incubated for 30 min, followed by measuring the fluorescence intensity at excitation 500 nm and emission 530 nm (Wadhwa, M. S., Collard, W. T., Adami, R. C., McKenzie, D. L., and Rice, K. G. (1997) Peptide-mediated gene delivery: influence of peptide structure on gene expression. Bioconjugate Chemistry 8, 81-8). The percent fluorescence intensity relative to control was plotted versus the stoichiometry of glycopeptide or peptide added to DNA to determine the equivalence point.

Glycopeptide DNA Polyplex Binding to DC-SIGN (+) and (−) CHO Cells

pGL3 plasmid (5 μg) was labeled with Label IT according to the manufacturer's instructions to prepare Cy5-DNA. The Man 9 glycopeptide (0.5 nmol) was combined with 1 μg Cy5-DNA in 60 μl of water and incubated for 30 min to form a polyplex. DC-SIGN (+) and (−) CHO cells (1×10⁶) (Granelli-Pipemo, A., Pritsker, A., Pack, M., Shimeliovich, I., Arrighi, J. F., Park, C. G., Trumpfheller, C., Piguet, V., Moran, T. M., and Steinman, R. M. (2005) Dendritic Cell Specific Intercellular Adhesion Molecule 3-Grabbing Nonintegrin/CD209 Is Abundant on Macrophages in the Normal Human Lymph Node and Is Not Required for Dendritic Cell Stimulation of the Mixed Leukocyte Reaction. J Immunology 7, 4260-4273) were each plated in triplicate in 35 mm wells and grown in DMEM supplemented with 7% FBS and 1% nonessential amino acids for 24 hrs. CHO (+) and (−) cells were transfected for 24 hrs with 1 μg of Cy5-DNA polyplex. The CHO cells were lifted by treatment with 5 ml of 1 mM EDTA in PBS and concentrated by centrifugation by 2,000×g, before diluting to a final volume of 1 ml in PBS. The transfected CHO (+) and (−) cells were analyzed by FACS on a Becton Dickson LSRII with excitation 488 nm and emission 575 nm, and compared to untransfected CHO cells.

In Vitro Transfection with Glycopeptide DNA Polyplexes

DC-SIGN (+) CHO cells (1×10⁵) were plated in 6-well plates in triplicate in 1 ml DMEM high glucose supplemented with 7% FBS and 1% MEM non-essential amino acids. After incubation for 24 hrs at 37° C. in 5% CO₂, the media was removed, the cells were washed with 1 ml DPBS, replaced with 1 ml DMEM high glucose media supplemented with 2% FBS and 0.3% MEM non-essential amino acids.

Cells were transfected in triplicate for 24 hrs with pGL3 polyplexes (1 μg DNA and 0.6 nmol of glycopeptide in a total volume of 30 μl). The efficiency of glycopeptide DNA polyplex transfection was compared with PEI-DNA transfection performed by combining 1 μg DNA with 1.2 μg of PEI (N:P of 9) in 100 μl of 5 mM Hepes, 270 mM mannitol, pH 7.4. After 24 hrs, cells were washed with 2 ml of DPBS and lysed with 0.5 ml of lysis buffer (25 mM tris chloride, 1 mM EDTA, 8 mM magnesium chloride, 1% Triton X-100, pH 7.8) for 10 min at 4° C. Cell lysates were scraped, transferred to 1.5 ml microcentrifuge tubes, and centrifuged for 10 mM at 13,000 g (at 4° C.) to pellet cell debris. Lysis buffer (400 μl) and ATP (4.3 μl of a 165 mM solution at pH 7) were combined in a test tube, mixed briefly, and placed in the luminometer. The relative light units were determined with 10 sec integration after automatic injection of 100 μl of 0.43 mM D-luciferin. Protein concentrations were measured by a BCA assay with bovine serum albumin as a standard (Smith, P. K., Krohn, R. I., Hermanson, G. T., Mallia, A. K., Gartner, F. H., Provenzano, M. D., Fujimoto, E. K., Goeke, N. M., Olson, B. J., and Klenk, D. C. (1985) Measurement of protein using bicinchoninic acid. Anal Biochem 150, 76-85). The amount of luciferase recovered in each sample was normalized to mg of protein and reported as the mean with standard deviation obtained from triplicate transfections.

Results

A strategy was developed to reversibly bind a high mannose N-glycan to plasmid DNA for the purpose of targeted uptake and expression of a reporter gene in cells expressing the DC-SIGN receptor. While in the present study, glycopeptide mediated gene transfer was only tested in a model cell system, the ultimate purpose of this gene delivery system would be to selectively transfect dendritic cells in humans to improve the efficacy of DNA vaccines.

The polyacridine peptide was designed to reversibly bind to plasmid DNA by a combination of intercalation. The solid phase synthesis of Cys-(Acr-Lys)₄ (SEQ ID NO: 23) necessitated the large scale synthesis of Fmoc-Lys(Acr) as an unnatural amino acid. Using this approach, a high affinity polyacridine peptide containing an N-terminal Cys was obtained in good yield.

The high-mannose N-glycan is a known ligand for DC-SIGN receptor and is the only N-glycan present on SBA. Because SBA is a galactose specific lectin, it is readily purified in a single affinity chromatography step. The affinity purification of SBA is essentially the same as first reported by (Sharon L is, H., Sharon, N., and Katchalski, E. (1966) Soybean Hemagglutinin, a Plant Glycoprotein. I. Isolation of a Glycopeptide. J. Biol. Chem. 241, 684-689), with the exception that the affinity resin was prepared by attaching galactosamine to 6-aminohexanoic acid functionalized Sepharose resin prepared using CDI as reported by Bethel and Ayers (Bethell, G. S., Ayers, J. S., Hancock, W. S., and Hearn, M. T. (1979) A novel method of activation of cross-linked agarose with 1,1′-carbonyldiimidazole which gives a matrix for affinity chromatography devoid of additional charged groups. J Biol Chem 254, 2572-4). This approach afforded the scalable preparation of the column resin without the use of cyanogen bromide.

The high-mannose N-glycan on SBA is not easily released by direct treatment with N-glycosidase F. Likewise, affinity purified SBA is very resistant to digestion with trypsin. Consequently, SBA is most efficiently converted to peptides by prolonged pronase digestion which produces the Man 9 N-glycan possessing a single Asn. Starting with 10 g of soy flour, the resulting Man 9 N-glycan was purified in good yield (6.7 μmols, 45%) using a combination of gel filtration and cation exchange chromatography as described (L is, H., Sharon, N., and Katchalski, E. (1966) Soybean Hemagglutinin, a Plant Glycoprotein. I. Isolation of a Glycopeptide. J. Biol. Chem. 241, 684-689; Gordon, J. A., Blumberg, S., Lis, H., and Sharon, N. (1972) Purification of Soybean Agglutinin by Affinity Chromatography of Sepharose-N-e-Aminocaproyl-B-D-Galactopyranosylamine. Febs Letters 24, 193-196). High field ¹H-NMR analysis of the purified Man 9 N-glycan established the presence of 9 mannose residues based on the resonance frequency of the anomeric proton for each. MALDI-TOF analysis was also consistent with the isolation of Man 9 N-glycan containing a single Asn residue.

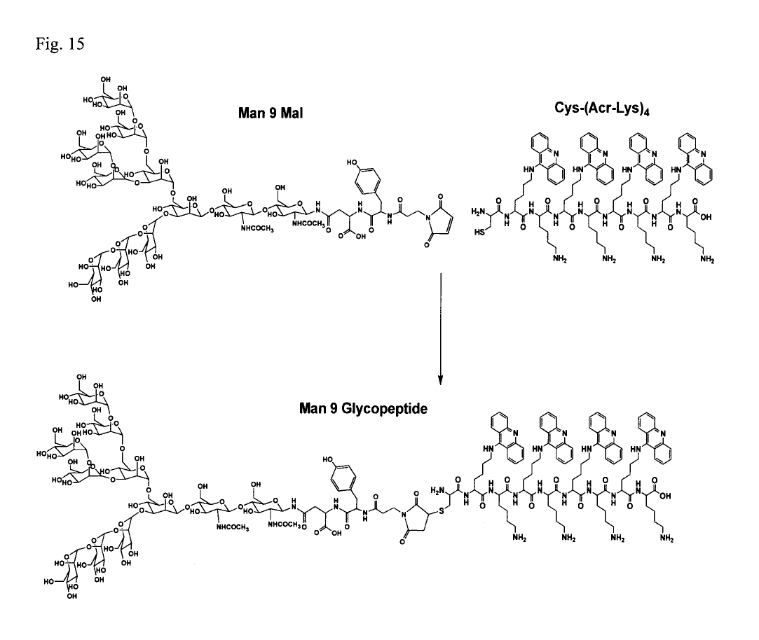

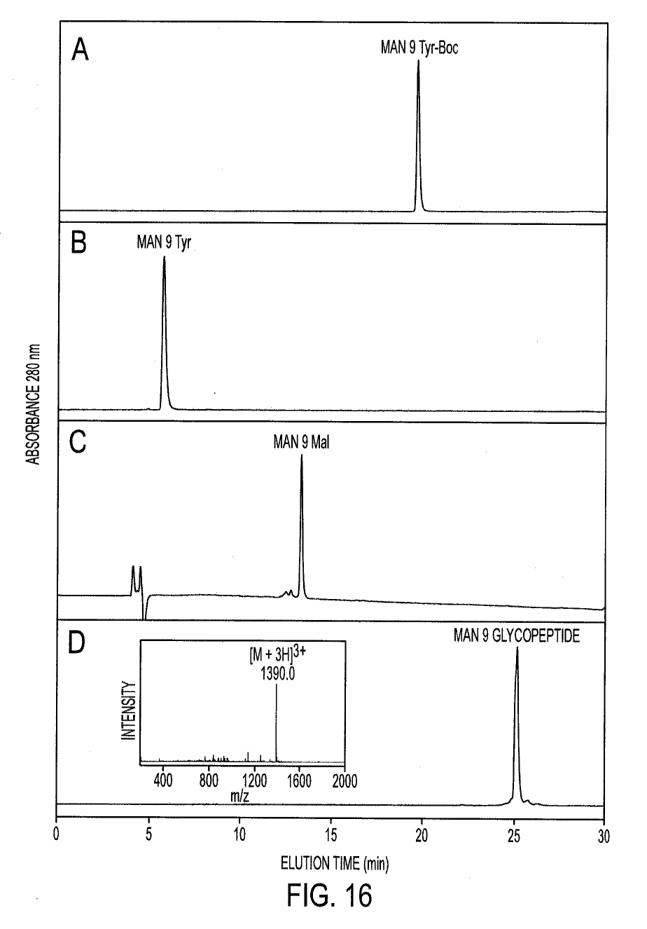

The conjugation of the Man 9 N-glycan to Cys-(Acr-Lys)₄ (SEQ ID NO: 23) required functionalization through the Asn residue (FIG. 15). To facilitate reaction monitoring by RP-HPLC, the Man 9 N-glycan was first modified by attaching Boc-Tyr to the N-terminus. Analysis of the Man 9 Tyr-Boc obtained from gel filtration chromatography revealed a single peak on analytical RP-HPLC with retention time of 19 min when detected by Abs₂₈₀ₙₘ (FIG. 16A). Removal of the Boc group by treatment with TFA resulted in a shift to an earlier retention time, confirming complete removal of Boc (FIG. 16B). The resulting amine terminus on Tyr was then modified with a propionate maleimide to provide a functional group that reacts with the Cys residue on Cys-(Acr-Lys)₄ (SEQ ID NO: 23). The resulting Man 9 Mal N-glycan product was also purified by gel filtration, and produced a single product when analyzed on RP-HPLC (FIG. 16C). Each N-glycan product described produced ESI-MS consistent with the reported structure.

To complete the synthesis of the Man 9 glycopeptide, the Man 9 Mal was reacted with nearly a stoichiometric amount of Cys-(Acr-Lys)₄ (SEQ ID NO: 23) (FIG. 15). In addition to the desired glycopeptide product, a dimeric peptide product formed. The RP-HPLC analysis of the purified Man 9 glycopeptide established its high purity and produced an ESI-MS consistent with the reported structure (FIG. 16D, inset).

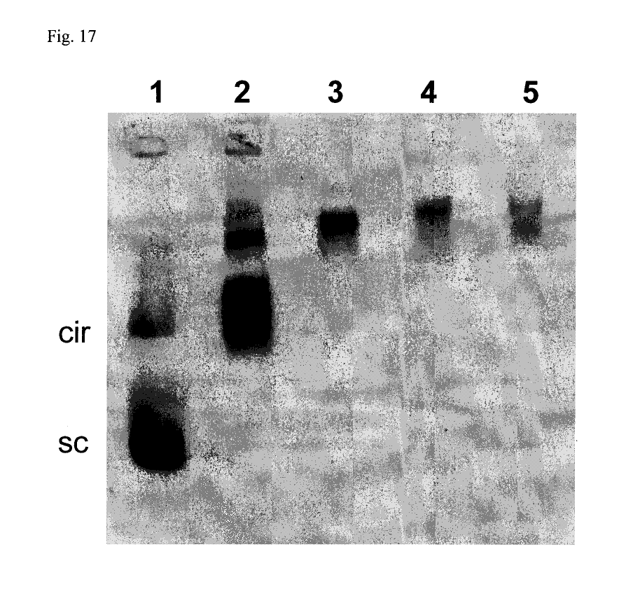

To examine the binding of the Man 9 glycopeptide to DNA, an agarose gel electrophoresis band shift assay was used. The electrophoretic migration of DNA was significantly retarded at increasing glycopeptide to DNA stoichiometry such that at 0.4 nmol of glycopeptide per μg of DNA or higher the band shift was complete (FIG. 17). In addition, the DNA bands were not visible by incorporating ethidium bromide into the gel prior to electrophoresis. Only post-staining with ethidium bromide following electrophoresis resulted in displacement of the polyacridine containing glycopeptides and the detection of DNA.

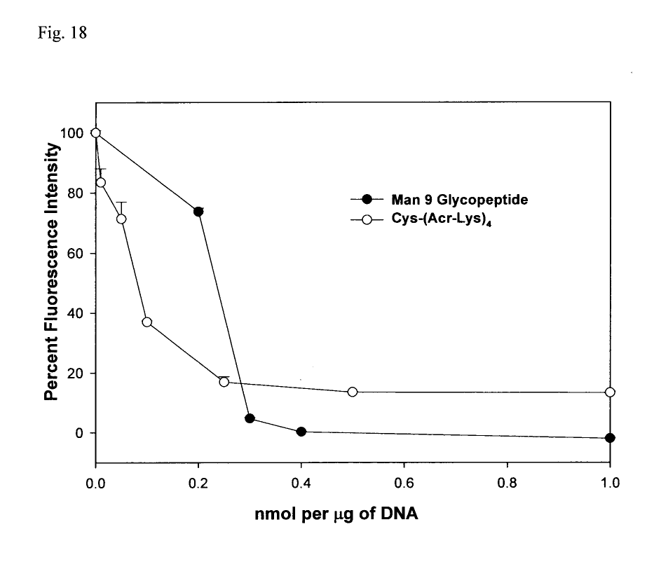

An additional analysis of glycopeptide DNA binding affinity utilized a fluorophore displacement assay. Increasing the stoichiometry of Man 9 glycopeptide to DNA resulted in the displacement of thiazole orange from DNA as determined by a decrease in fluorescence intensity. An asymptote in the fluorescence intensity at 0.4 nmol Man 9 glycopeptide per 1 μg DNA established this as the equivalence point for polyplex formation (FIG. 18). By comparison, Cys-(Acr-Lys)₄ (SEQ ID NO: 23) appears to have slightly greater DNA binding affinity. Based on this and the results in FIG. 17, a stoichiometry of 0.5 nmol per μg of DNA was used to prepare polyplexes and evaluate cell uptake and in vitro gene transfer.

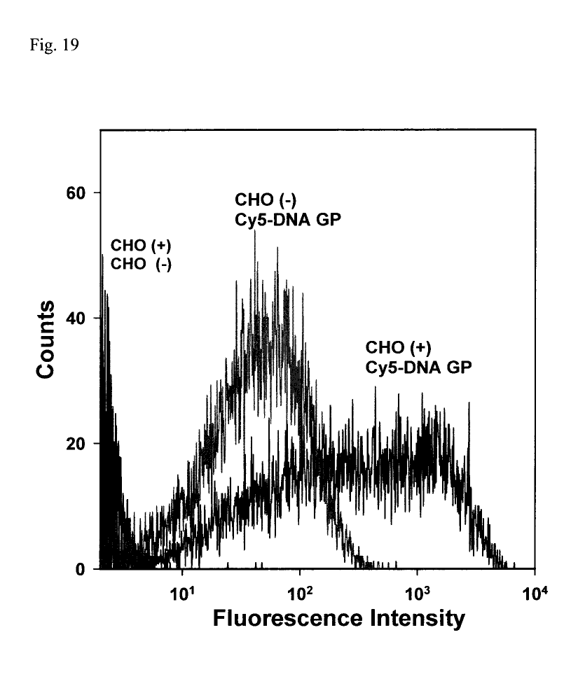

The uptake of Man 9 glycopeptide Cy5-DNA polyplexes into CHO cells, either with (+) or without (−) stably transfected DC-SIGN receptor, was assayed by FACS analysis (FIG. 19). A 24 hr 37° C. incubation of Man 9 glycopeptide Cy5-DNA polyplexes with CHO (+) and (−) cells was used to simulate conditions used for in vitro transfection. FACS analysis revealed uptake of Man 9 glycopeptide Cy5-DNA polyplexes in both CHO (+) and (−) cells relative to untreated cells. CHO (−) cells were able to bind Man 9 glycopeptide Cy5-DNA polyplexes non-specifically through ionic interaction. However, the cell uptake in CHO (+) cells was 10-50 fold higher than CHO (−) cells based on the intensity of fluorescence (FIG. 19). The results suggest that DC-SIGN on CHO (+) cells increases the uptake of Man 9 glycopeptide Cy5-DNA polyplexes by receptor mediated endocytosis.

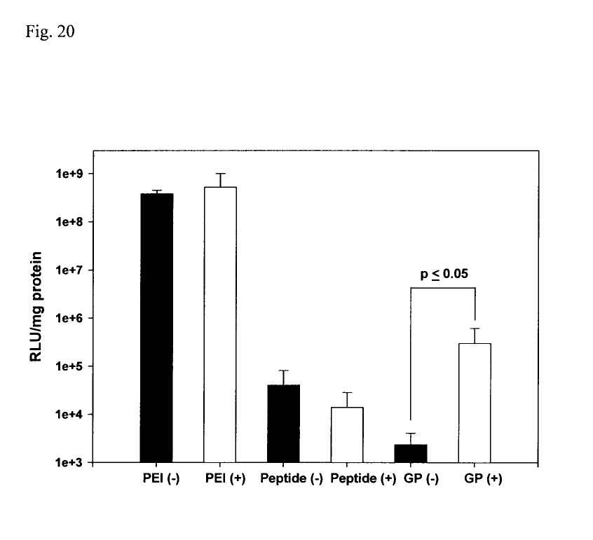

To determine if the increased uptake of Man 9 glycopeptide DNA polyplexes by CHO (+) cells results in increased gene expression, a series of gene transfer experiments were performed in CHO (+) and (−) cells. The results established that PEI-DNA polyplexes were the most efficient in gene transfer but were not selective for (+) or (−) cells (FIG. 20). The Cys-(Acr-Lys)₄ peptide (SEQ ID NO: 23) mediated low level gene expression in CHO cells, but did not demonstrate selectivity. In contrast Man 9 glycopeptide DNA polyplexes mediated approximately 100-fold greater gene expression in CHO (+) cells relative to an identical experiment conducted in CHO (−) cells (FIG. 20). These results are consistent with the cell uptake studies and support the hypothesis that the DC-SIGN receptor can facilitate receptor mediated endocytosis of DNA polyplexes.

Discussion

The clinical application of DNA vaccines for the treatment of disease is currently limited by the low efficiency of nonviral delivery systems. While it will be possible to optimize the DNA vector, it is also necessary to improve the delivery system. A large body of growing evidence suggests that targeting of DNA, as well as adjuvant, to enter dendritic cells leads to an enhanced immune response. It has been demonstrated that DC-SIGN is an endocytosing, cell surface C-type lectin that can be used to target proteins and DNA into dendritic cells. Given that the specificity of DC-SIGN is for mannose or Le^{X} on N-glycans, we explored an approach to reversibly attach a high-mannose N-glycan ligand to plasmid DNA to establish DNA uptake and gene expression in cells expressing DC-SIGN.

To achieve this, a purification of a high-mannose N-glycan from soybean agglutinin was used. This required the development of a large-scale galactose affinity column by applying a carbodiimide coupling strategy. The purified N-glycan was then systematically modified on the Asn N-terminus to allow coupling to a DNA binding peptide. The conjugation of Boc-Tyr resulted in introduction of a hydrophobic chromophore that could be iodinated. Removal of Boc proceeded in neat TFA to afford the Tyr amine. Modification of the amine with a propionate possessing a maleimide group resulted in a Man 9 Mal that reacted with a thiol on the peptide.

The polyacridine peptide was modeled after similar peptides first reported by Ueyama (Ueyama, H., Takagi, M., Waki, M., and Takenaka, S. (2001) DNA binding behavior of peptides carrying acridinyl units: First example of effective poly-intercalation. NUCLEIC ACIDS SYMP SER (OXF) 1, 163-164). However, a Cys residue was included on the N-terminus to allow specific coupling to Man 9 Mal. This conjugation led to a Man 9 glycopeptide that possessed affinity for DC-SIGN and DNA. The DNA binding affinity of a polyacridine peptide is related to the number of acridines and the identity of the spacing amino acid. Two different experiments established that the glycopeptide binds to DNA. A band shift assay and a fluorophore displacement assay both confirmed that the Man 9 glycopeptide binds maximally to DNA at 0.4 nmol per μg of DNA. The binding affinity for the unglycosylated peptide was slightly greater than that of the glycopeptide.

The ability of DC-SIGN to bind to Man 9 glycopeptide DNA polyplexes was established using a CHO cell line stably transfected with the human DC-SIGN receptor. The results of FACS analysis using Cy5-DNA glycopeptide polyplexes confirmed preferential binding to CHO (+) cells compared to CHO (−) cells. It should be noted that Cy5-DNA glycopeptide polyplexes bind weakly to CHO (−) cells perhaps due to the cationic nature of the polyplexes.

The results of FACs analysis compare favorably with those of gene transfer studies. A 100-fold increase in gene transfer was determined when transfecting CHO (+) cells with glycopeptide DNA polyplexes compared to the same transfection of CHO (−) cells. Unglycosylated peptide formed cationic polyplexes that mediated non-specific gene transfer. While the levels of gene transfer are much lower than that mediated by PEI DNA, the selectivity of Man 9 glycopeptide mediated gene transfer support the hypothesis that DC-SIGN binds the ligand and transports the DNA polyplex into the cell.

Taken together, this study demonstrates the first application of a high-mannose N-glycan to mediated gene transfer via DC-SIGN, and demonstrates the application of a polyacridine anchor peptide to reversibly bind Man 9 to DNA. These results may be expanded to eventually improve the efficacy of DNA vaccines for use in humans.

### Example 4

### Metabolically Stabilized Long-Circulating PEGylated Polyacridine Peptide Polyplexes Mediate Hydrodynamically Stimulated Gene Expression in Liver

A new class of gene delivery peptides were developed and tested in mice. The peptides of the general structure (Acr-X)ₙ, possessed from 2-6 repeats of a modified Lys, with acridine (Acr) attached to the c-amine of Lys, spaced by amino acid X (SEQ ID NO: 89). (Fernandez, C. A. Baumhover, N. J., Duskey, J., Khangharia, S., Kizzire, K., Ericson, M., and Rice, K. G. “Metabolically Stabilized Long-Circulating PEGylated Polyacridine Peptide Polyplexes Mediate Hydrodynamically Stimulated Gene Expression in Liver” Gene Therapy (2011) 18, 23-37.) The number of Acr repeats and the identity of the spacing amino acid were studied in relation to the DNA binding affinity, metabolic stability, pharmacokinetic half-life and in vivo gene transfer efficiency. Each peptide possessed a C-terminal Cys that was modified either reversibly or irreversibly with polyethylene glycol (PEG_{5000 Da}). PEGylated polyacridine peptides were examined for DNA binding affinity by dye displacement and gel band shift, for particle size and zeta potential using QELS, and for shape using atomic force microscopy (AFM). Substitution of (Acr-X)₄-Cys-PEG, with X as either Lys, Arg, Leu or Glu (SEQ ID NO: 90), established that polyacridine peptides possessing a Lys and Arg bind with higher affinity than peptides possessing Leu and Glu. Increasing the number of Acr-Lys repeats from 2 to 6 resulted in increased DNA binding affinity and greater protection of DNA from DNase. Pharmacokinetic analysis of PEGylated (Acr-Lys)₆ polyplexes (SEQ ID NO: 33) established the metabolic stability of plasmid DNA with a long-circulatory half-life of 3 hrs in blood. A high level of luciferase expression was stimulated in the liver following a tail vein dose of PEGylated polyacridine peptide pGL3 polyplexes (1 μg in 50 μl). Administration of the stimulatory hydrodynamic dose of normal saline at times ranging from 5-60 min post-DNA administration, resulted in liver expression that was equal or greater than the level mediated by direct hydrodynamic dosing of pGL3 (1 μg). The results establish the unique properties of PEGylated polyacridine peptides that facilitate reversible binding to plasmid DNA, protect it from DNase in vivo, allow it to circulate for hours, and release transfection-competent DNA into the liver to mediate high-level gene expression.

Introduction

New gene delivery agents are needed that function by efficiently mediating targeted nonviral gene delivery in vivo. Some of the most successful delivery agents developed to date, such as PEI and cationic lipids, produce robust gene transfer in vitro, but fail to mediate significant gene expression in vivo. In contrast, hydrodynamic (HD) dosing and electroporation are proven physical methods that are highly efficient in delivering plasmid DNA to animals and may find utility in humans. However, due to the invasiveness of HD dosing, there is a need for new gene delivery agents that produce gene expression at levels comparable to HD dosing, but without the requirement of high-volume administration.

Most i.v. dosed nonviral gene delivery systems are cationic polyplexes or lipoplexes, the primary exception being anionic liposomes with encapsulated DNA. To increase their compatibility with blood, cationic polyplexes are modified with polyethylene glycol (PEG) to block albumin binding and to “stealth” their rapid endocytosis by cells of the reticuloendothelial system (RES). Despite these attempts, most PEGylated cationic polyplexes still quickly biodistribute to the liver, and are taken-up primarily by Kupffer cells. Few, if any, of these experimental i.v. dosed gene delivery systems have produced appreciable gene expression in the liver, especially when compared to the level of luciferase expression from an equivalent dose of DNA delivered hydrodynamically.

To develop nonviral gene delivery agents that function with greater efficiency in vivo following i.v. dosing, it is necessary to control the size, charge and metabolic stability of polyplexes. The cationic surface charge of most polyplexes results from residual unpaired amines on the cationic polymer used to condense DNA. Anionic polyplexes may be more blood compatible, but would require a new mode of binding polymers to DNA, not based on electrostatic interaction.

The use of polyintercalation has been shown to increase the binding affinity of small polymers and peptides to DNA. Previous studies from our group have established that polyacridine peptides possessing four Lys(Acr) residues (ε amine of Lys is modified with acridine), spaced by either a Lys or Arg, produce high affinity binding for plasmid DNA. Modification of polyacridine peptides with either a fusogenic peptide (melittin) or an N-glycan (high-mannose) produced gene transfer agents that formed cationic polyplexes resulting in high level selective gene transfer in vitro. PEGylated polyacridine peptides have been shown to bind to plasmid DNA and produce unique, metabolically stable, anionic open-polyplexes that are capable of mediating gene transfer in vivo when delivered by i.m.-electroporation.

In the present study we prepared a panel of PEGylated polyacridine peptides to establish their utility in mediating gene expression following i.v. dosing. The results establish a clear structure-activity relationship by which protection of the DNA in blood and liver by a PEGylated polyacridine peptide results in the ability to stimulate high level gene expression in liver with a delayed hydrodynamic dose of saline. The results also establish the ability to extend the circulatory half-life of a significant percentage of i.v. dosed DNA, which is an important prerequisite of achieving targeted gene delivery to organs and tissues. The synthetic adaptability of polyacridine peptides, along with the ability to prepare either anionic-open or cationic-closed-polyplexes that mediate gene transfer in vivo, demonstrate the unique importance of poly-intercalative binding to achieve gene delivery.

Materials and Methods

Unsubstituted Wang resin, 9-hydroxybenzotriazole, Fmoc-protected amino acids, O-(7-Azabenzo-triazol-1-yl)-N,N,N′,N′-tetramethylurionium hexafluorophosphate (HATU), Fmoc-Lysine-OH, and N-Methyl-2-pyrrolidinone (NMP) were obtained from Advanced ChemTech (Lexington, Ky.). N,N-Dimethylformamide (DMF), trifluoroacetic acid (TFA), and acetonitrile were purchased from Fisher Scientific (Pittsburgh, Pa.). Diisopropylethylamine, piperidine, acetic anhydride, Tris(2-carboxyethyl)-phosphine hydrochloride (TCEP), 9-chloroacridine and thiazole orange were obtained from Sigma Chemical Co. (St. Louis, Mo.). Agarose was obtained from Gibco-BRL. mPEG-maleimide and mPEG-OPSS (5,000 Da) were purchased from Laysen Bio (Avab, Ala.). D-Luciferin and luciferase from Photinus pyralis were obtained from Roche Applied Science (Indianapolis, Ind.). pGL3 control vector, a 5.3 kb luciferase plasmid containing a SV40 promoter and enhancer, was obtained from Promega (Madison, Wis.). pGL3 was amplified in a DH5α strain of Escherichia coli and purified according to manufacturer's instructions.

Synthesis and Characterization of Polyacridine Peptides

9-Phenoxyacridine and Fmoc-Lysine(Acridine)-OH were prepared as recently reported (Baumhover N J, Anderson K, Fernandez C A, Rice K G. Synthesis and In Vitro Testing of New Potent Polyacridine-Melittin Gene Delivery Peptides, Bioconjugate Chemistry 2010; 21: 74-86; Anderson, K, Fernandez, C. A., and Rice, K. G. “N-Glycan Targeted Gene Delivery to the Dendritic Cell SIGN Receptor” Bioconjugate Chemistry (2010) 21, 1479-85). The polyacridine peptides reported in Table 1 were prepared by solid phase peptide synthesis on a 30 μmol scale on an APEX 396 Synthesizer using standard Fmoc procedures including 9-hydroxybenzotriazole and HATU activation while employing double coupling of Fmoc-Lys(Acr)-OH and triple coupling for the spacing amino acid while using a 5-fold excess of amino acid over resin and omitting N-capping of truncated peptide species.

TABLE 4
PEGylated Polyacridine Peptides

| Polyacridine Peptides | SEQ ID NO: | Mass (calc/obs)^{a} | % Yield |
|---|---|---|---|
| (Acr-Lys)₂-Cys | 91 | 988.5/988.3 | 37 |
| (Acr-Lys)₄-Cys | 71 | 1855.3/1855.1 | 26 |
| (Acr-Lys)₆-Cys | 92 | 2722.4/2722.0 | 20 |
| (Acr-Arg)₄-Cys | 11 | 1967.4/1967.2 | 30 |
| (Acr-Leu)₄-Cys | 72 | 1795.3/1795.1 | 31 |
| (Acr-Glu)₄-Cys | 93 | 1859.1/1859.0 | 22 |
| PEGylated Polyacridine | | | |
| Peptides | SEQ ID NO: | Mass (calc/obs)^{b} | % Yield |
| PEGylated-Mal-(Acr-Lys)₂ | 94 | 6488/6531 | 64 |
| PEGylated-Mal-(Acr-Lys)₄ | 95 | 7355/7218 | 55 |
| PEGyalted-Mal-(Acr-Lys)₆ | 96 | 8222/8116 | 66 |
| PEGylated-Mal-(Acr-Arg)₄ | 24 | 7467/7218 | 53 |
| PEGyalted-SS-(Acr-Arg)₄ | 25 | 7467/7450 | 44 |
| PEGyalted-Mal-(Acr-Leu)₄ | 97 | 7295/7110 | 46 |
| PEGyalted-Mal-(Acr-Glu)₄ | 98 | 7359/7262 | 35 |

^{a}Determined by ESI-MS.
^{b}Determined by MALDI-TOF MS.

Peptides were removed from resin and side chain deprotected using a cleavage cocktail of TFA/ethanedithiol/water (93:4:3 v/v/v) for 3 hrs followed by precipitation in cold ether. Precipitates were centrifuged for 10 min at 5000×g at 4° C. and the supernatant decanted. Peptides were then reconstituted with 0.1 v/v % TFA and purified to homogeneity on RP-HPLC by injecting 0.5-2 μmol onto a Vydac C18 semi-preparative column (2×25 cm) eluted at 5 ml per min with 0.1 v/v % TFA with an acetonitrile gradient of 20-30 v/v % over 30 min while monitoring acridine at 409 nm. The major peak was collected and pooled from multiple runs, concentrated by rotary evaporation, lyophilized, and stored at −20° C. Purified peptides were reconstituted in 0.1 v/v % TFA and quantified by absorbance (acridine ε_{409 nm}=9266 M⁻¹ cm⁻¹ assuming additivity of c for multiple acridines) to determine isolated yield (Table 4). Purified peptides were characterized by LC-MS by injecting 2 nmol onto a Vydac C18 analytical column (0.47×25 cm) eluted at 0.7 ml per min with 0.1 v/v % TFA and an acetonitrile gradient of 10-55 v/v % over 30 min while acquiring ESI-MS in the positive mode.

Synthesis and Characterization of PEGylated Polyacridine Peptides

PEGylation of the Cys residue on (Acr-X)ₙ-Cys was achieved by reacting with 1 mol of peptide with 1.1 mol of PEG_{5000 Da}-maleimide or PEG_{5000 Da}-OPSS in 4 ml of 10 mM ammonium acetate buffer pH 7 for 12 hrs at RT. PEGylated peptides were purified by semipreparative HPLC as previously described and eluted with 0.1 v/v % TFA with an acetonitrile gradient of 25-65 v/v % acetonitrile while monitoring acridine at 409 nm. The major peak was collected and pooled from multiple runs, concentrated by rotary evaporation, lyophilized, and stored at −20° C. Counter-ion exchange was accomplished by chromatography on a G-25 column (2.5×50 cm) equilibrated with 0.1 v/v % acetic acid to obtain the peptide in an acetate salt form. The major peak corresponding to the PEG-peptide eluted in the void volume (100 ml) was pooled, concentrated by rotary evaporation, and freeze-dried. PEG-peptides were reconstituted in water and quantified by Abs₄₀₉ₙₘ to determine isolated yield (Table 4). PEG-peptides were characterized by MALDI-TOF MS by combining 1 nmol with 10 μl of 2 mg per ml a-cyano-4-hydroxycinnamic acid (CHCA) in 50 v/v % acetonitrile and 0.1 v/v % TFA. Samples were spotted onto the target and ionized on a Bruker Biflex III Mass Spectrometer operated in the positive ion mode.

Formulation and Characterization of PEGylated Polyacridine Peptide Polyplexes

The relative binding affinity of PEGylated polyacridine peptides for DNA was determined by a fluorophore exclusion assay (Wadhwa M S et al. Peptide-mediated gene delivery: influence of peptide structure on gene expression. Bioconjugate Chemistry 1997; 8: 81-88). pGL3 (200 μl of 5 μg/ml in 5 mM Hepes pH 7.5 containing 0.1 μM thiazole orange) was combined with 0, 0.05, 0.1, 0.25, 0.35, 0.5, or 1 nmol of PEGylated polyacridine peptide in 300 of Hepes and allowed to bind at RT for 30 min. Thiazole orange fluorescence was measured using an LS50B fluorometer (Perkin-Elmer, U.K.) by exciting at 498 nm while monitoring emission at 546 nm with the slit widths set at 10 nm. A fluorescence blank of thiazole orange in the absence of DNA was subtracted from all values before data analysis. The data is presented as nmol of PEGylated polyacridine peptide per μg of DNA versus the percent fluorescence intensity±the standard deviation determined by three independent measurements.

The particle size and zeta potential were determined by preparing 2 ml of polyplex in 5 mM Hepes pH 7.5 at a DNA concentration of 30 μg per ml and a PEGylated polyacridine peptide stoichiometry of 0, 0.2, 0.3, 0.4, 0.5, 0.6, 0.8 or 1 nmol per μg of DNA. The particle size was measured by quasi-elastic light scattering (QELS) at a scatter angle of 90° on a Brookhaven ZetaPlus particle sizer (Brookhaven Instruments Corporation, NY). The zeta potential was determined as the mean of ten measurements immediately following acquisition of the particle size.

The shape of PEGylated polyacridine polyplexes were determined using atomic force microscopy (AFM). pGL3 alone, or anionic PEGylated polyacridine polyplexes at 0.2 nmol of peptide per μg of DNA, were prepared at a concentration of 100 μg per ml of DNA in 10 mM Tris, 1 mM EDTA pH 8. Polyplexes were diluted to 1 μg per ml in 40 mM Hepes 5 mM nickel chloride pH 6.7 and deposited on a fresh cleaved mica surface (cationic mica) for 10 min followed by washing with deionized water. Cationic PEGylated polyacridine polyplexes prepared at 0.8 nmol of PEGylated (Acr-Lys)₆ (SEQ ID NO: 33) per μg of DNA, at 100 μg per ml of DNA in 10 mM Tris, 1 mM EDTA pH 8 were deposited directly on a freshly cleaved mica surface (anionic mica) and allowed to bind for 10 min prior to washing with deionized water. Images were captured using an Asylum AFM MFP3D (Santa Barbara, Calif.) operated in the AC-mode using a silicon cantilever (Ultrasharp NSC15/AIBS, Mikro Masch).

Gel Band Shift and DNase Protection Assay

pGL3 (1 μg), or pGL3 polyplexes (1 μg) were prepared at either 0.2 or 0.8 nmol of PEGylated (Acr-Lys)_{2, 4, or 6} (SEQ ID NOS 32, 29, and 33, respectively), in 20 μl of 5 mM Hepes buffer pH 7.4. The ability of polyplexes to resist digestion with DNase was determined by incubation with 0.06 U of DNase I for 10 min. DNase was inactivated by the addition of 500 μl of 0.5 mg per ml proteinase K (in 100 mM sodium chloride, 1% SDS and 50 mM Tris pH 8.0) followed by incubation at 37° C. for 30 min. The polyplexes were extracted with 500 μl of phenol/chloroform/isoamyl alcohol (24:25:1) to remove PEGylated peptides, followed by precipitation of DNA with the addition of 1 ml of ethanol. The precipitate was collected by centrifugation at 13,000 g for 10 min, and the DNA pellet was dried and dissolved in 5 mM Hepes buffer pH 7.4. DNA samples were combined with 2 μl of loading buffer and applied to a 1% agarose gel (50 ml) and electrophoresed in TBE buffer at 70 V for 60 min (Adami R C, Rice K G. Metabolic stability of glutaraldehyde cross-linked peptide DNA condensates. Journal of Pharmaceutical Sciences 1999; 88: 739-746). The gel was post-stained with 0.1 μg per ml ethidium bromide at 4° C. overnight then imaged on a UVP Biospectrum Imaging System (Upland, Calif.).

Pharmacokinetic Analysis of PEGylated Polyacridine Polyplexes.

Radioiodinated pGL3 was prepared as previously described (Terebesi J, Kwok K Y, Rice K G. Iodinated plasmid DNA as a tool for studying gene delivery. Analytical Biochemistry 1998; 263: 120-123). Triplicate mice were anesthetized by i.p. injection of ketamine hydrochloride (100 mg per kg) and xylazine hydrochloride (10 mg per kg) then underwent a dual cannulation of the right and left jugular veins. An i.v. dose of ¹²⁵I-DNA (3 μg in 50 μl of HBM, 1.2 μCi) or ¹²⁵I-DNA polyplex (3 μg) was administered via the left catheter, and blood samples (10 μl) were drawn from the right catheter at 1, 3, 6, 10, 20, 30, 60, 90 and 120 min and immediately frozen, then replaced with 10 μl of normal saline. The amount of radioactivity in each blood time point was quantified by direct γ-counting followed by extraction of the DNA and separation by gel electrophoresis as described above. The gel was dried on a zeta probe membrane and autoradiographed on a Phosphor Imager (Molecular Devices, Sunnyvale Calif.) following a 15 hr exposure.

Biodistribution Analysis of PEGylated Polyacridine Polyplexes

Triplicate mice were anesthetized and a single catheter was placed in the left jugular vein. ¹²⁵I-DNA (1.5 μg in 50 μL of HBM, 0.6 μCi) or ¹²⁵I-DNA polyplexes (1.5 μg) were dosed i.v. followed by vein ligation. After 5, 15, 30, 60, or 120 min, mice were sacrificed by cervical dislocation and the major organs (liver, lung, spleen, stomach, kidney, heart, large intestine, and small intestine) were harvested, and rinsed with saline. The radioactivity in each organ was determined by direct γ-counting and expressed as the percent of the dose in the organ.

Hydrodynamic Stimulation and Bioluminescence Imaging

pGL3 (1 μg) was prepared in a volume of normal saline corresponding to 9 wt/vol % of the mouse's body weight (1.8-2.25 ml based on 20-25 g mice). The DNA dose was administered by tail vein to 4-5 mice in 5 sec according to a published procedure (Liu F, Song Y, Liu D. Hydrodynamics-based transfection in animals by systemic administration of plasmid DNA. Gene Ther 1999; 6: 1258-1266; Zhang G, Budker V, Wolff J A. High levels of foreign gene expression in hepatocytes after tail vein injections of naked plasmid DNA. Human Gene Therapy 1999; 10: 1735-1737). Mice (4-5) were also dosed tail vein with 1 μg of PEGylated polyacridine polyplex in 50 μl of HBM (5 mM Hepes, 0.27 M mannitol, pH 7.4). At times ranging from 5-120 min, a stimulatory hydrodynamic dose of normal saline (9 wt/vol % of the body weight) was administered over 5 sec. At 24 hrs post-DNA dose, mice were anesthetized by 3% isofluorane, then administered an i.p. dose of 80 μl (2.4 mg) of D-luciferin (30 μg/μl in phosphate-buffered saline). At 5 min following the D-luciferin dose, mice were imaged for bioluminescence (BLI) on an IVIS Imaging 200 Series (Xenogen). BLI was performed in a light-tight chamber on a temperature-controlled, adjustable stage while isofluorane was administered by a gas manifold at a flow rate of 3%. Images were acquired at a ‘medium’ binning level and a 20 cm field of view. Acquisition times were varied (1 sec⁻¹ min) depending on the intensity of the luminescence. The Xenogen system reported bioluminescence as photons/sec/cm²/seradian in a 2.86 cm diameter region of interest covering the liver. The integration area was transformed to pmols of luciferase in the liver using a previously reported standard curve (Rettig G et al. Quantitative Bioluminscence Imaging of Transgene Expression In Vivo. Analytical Biochemistry 2006; 335: 90-94). The results were determined to be statistically significant (p≦0.05) based on a two-tailed unpaired t-test.

Results

A panel of PEGylated polyacridine peptides were prepared and tested for their ability to bind and transport DNA in vivo. The peptides were designed to test the influence of DNA binding affinity on gene transfer efficiency. To examine the influence of spacing amino acid, four peptides of the general structure (Acr-X)₄ were prepared, where X is either Lys, Arg, Leu or Glu (SEQ ID NO: 99) (Table 4). As discussed in more detail below, a Lys spacing amino acid proved most efficient in gene transfer, which warranted an expansion of (Acr-Lys)ₙ to include repeats of 2 and 6 (SEQ ID NO: 100) (Table 4). Each polyacridine peptide also possessed a C-terminal Cys residue to allowed modification with PEG. During the course of synthesis, the synthetic yields of polyacridine peptides were improved by using HATU to activate amino acids during coupling. Improved yields were also the result of applying triple coupling of the spacing amino acids, omitting N-capping to avoid a side reaction with acridine, and using ethanedithiol as a scavenger during workup. Under these conditions, (Acr-Lys)ₙ with repeats ranging from 2 to 6 (SEQ ID NO: 101) were prepared in 20-37% isolated yield (Table 1). Each polyacridine peptide produced an ESI-MS consistent with the calculated mass (Table 4).

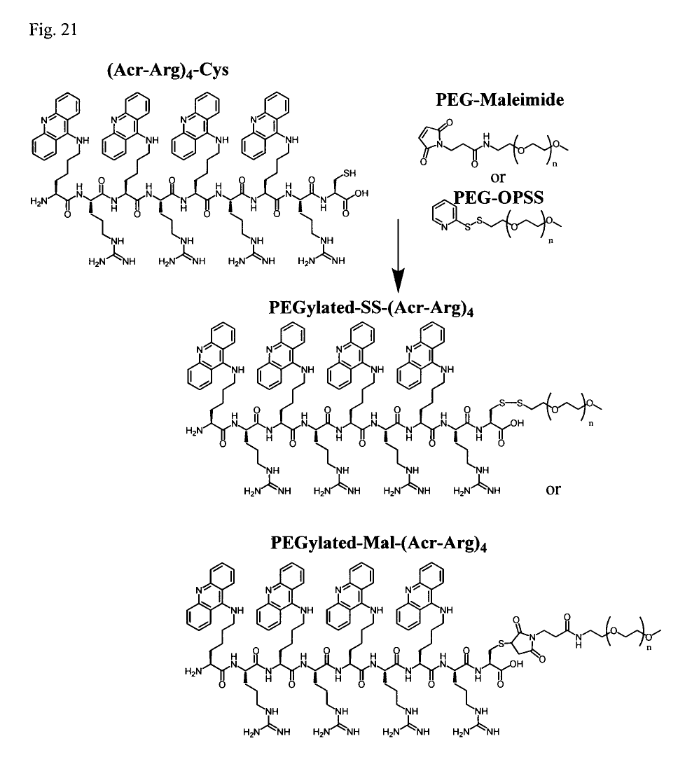

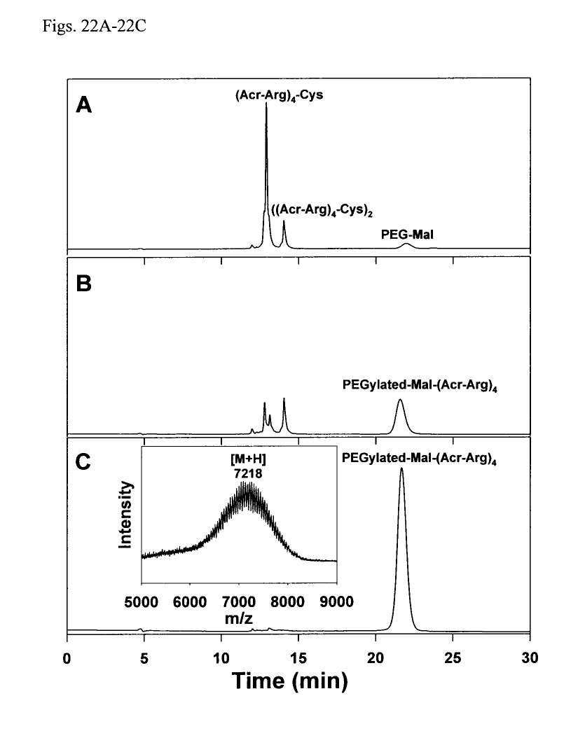

Conjugation of each polyacridine peptide with PEG was accomplished by reaction of the Cys residues with either PEG-maleimide or PEG-OPSS, resulting in PEGylated polyacridine peptides with a reducible disulfide or a non-reducible maleimide linkage (FIG. 21). The reaction was monitored by RP-HPLC in which the polyacridine peptide (FIG. 22A) was mostly consumed, resulting in formation of a later eluting PEG-peptide (FIG. 22B). Preparative RP-HPLC purification produced PEG peptides free of unreacted polyacridine peptide and PEG as established by analytical RP-HPLC and MALDI-TOF analysis (FIG. 22C). Each PEGylated polyacridine peptide produced a MALDI-TOF MS with multiple peaks due to the polydispersity of PEG_{5000 Da}, with average mass closely correlated to the calculated mass for the PEGylated peptide (Table 4).

The relative binding affinity of PEGylated polyacridine peptides for pGL3 was compared by determining the concentration of peptide that displaces a thiazole orange intercalator dye, resulting in decreased fluorescence.

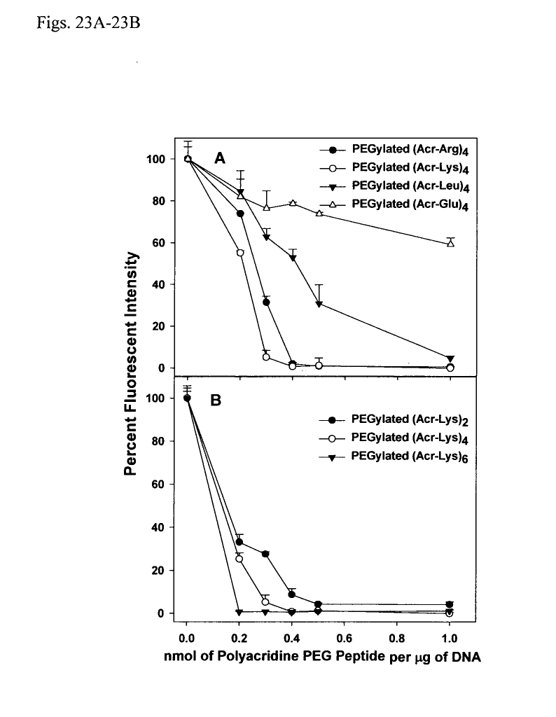

Comparison of PEGylated (Acr-X)₄ (X is either Arg, Lys, Leu or Glu (SEQ ID NOS 28-31, respectively)) established the importance of cationic amino acids to increase binding affinity. Both the Lys and Arg analogue demonstrated high affinity by completely displacing thiazole orange at 0.4 nmol of peptide per μg of DNA. Conversely, the Leu analogue demonstrated weaker binding, resulting in full displacement at 1 nmol, and the Glu analogue was determined to have the lowest affinity by only achieving 40% displacement at 1 nmol (FIG. 23A).

A similar comparison of the binding affinities for PEGylated (Acr-Lys)_{2, 4 and 6} (SEQ ID NOS 32, 29, and 33, respectively)established a relationship of increasing affinity with increasing the number of Acr, such that PEGylated (Acr-Lys)₆ (SEQ ID NO: 33)completely displaced thiazole orange at 0.2 nmols of peptide per μg of DNA (FIG. 23B). The importance of the spacing amino acid was further established by PEGylated (Acr-Lys)₂ (SEQ ID NO: 32) which possessed greater DNA binding affinity than PEGylated (Acr-Glu)₄ (SEQ ID NO: 31) (FIGS. 23A and B).

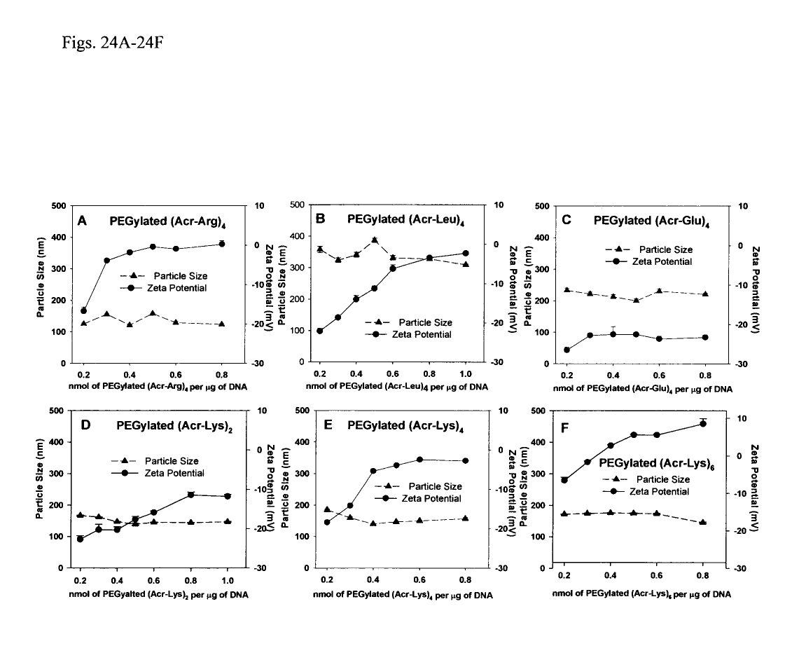

The size and charge of DNA polyplexes are often a function of the stoichiometry of peptide bound to DNA. To examine this relationship for PEGylated polyacridine polyplexes, QELS particle size and zeta potential were measured as a function of peptide to DNA ratio. One unusual property of PEGylated polyacridine polyplexes was their QELS mean diameter remained constant throughout the titration (FIGS. 24A-24F). Each of the polyplexes had an apparent mean diameter of approximately 100-200 nm, with the exception of PEGylated (Acr-Leu)₄ (SEQ ID NO: 30) and PEGylated (Acr-Glu)₄ (SEQ ID NO: 31) which produced polyplexes of an 200-400 nm mean diameter (FIGS. 24B and C).

A second unusual property of PEGylated polyacridine polyplexes was the observed zeta potential at each peptide stoichiometry (FIGS. 24A-24F). Titration with PEGylated (Acr-Arg)₄ (SEQ ID NO: 28) resulted in an increase in zeta potential from −15 mV to a maximum of −2 mV at 0.4 nmols of peptide per μg of DNA or higher (FIG. 24A). Similar zeta potential titration curves were determined for PEGylated (Acr-Leu)₄, (Acr-Lys)₂ and (Acr-Lys)₄ (SEQ ID NOS 30, 32, and 29, respectively), each of which resulted in polyplexes with a negative zeta potential when fully titrated (FIGS. 24B, 24D and 24E). In contrast, PEGylated (Acr-Glu)₄ (SEQ ID NO: 31) produced no change in zeta potential during the titration (FIG. 24C), and titration with PEGylated (Acr-Lys)₆ (SEQ ID NO: 33) produced zeta potentials that increased from −5 mV to a maximum of +5 mV during the titration (FIG. 24F). These data suggested that, unlike polylysine or PEI polyplexes that condense DNA into smaller polyplexes of 50-100 nm with charge of +25 mV, polyacridine peptides bind without dramatically changing DNA size and can be, in certain cases, titrated to completion while maintaining a negative zeta potential.

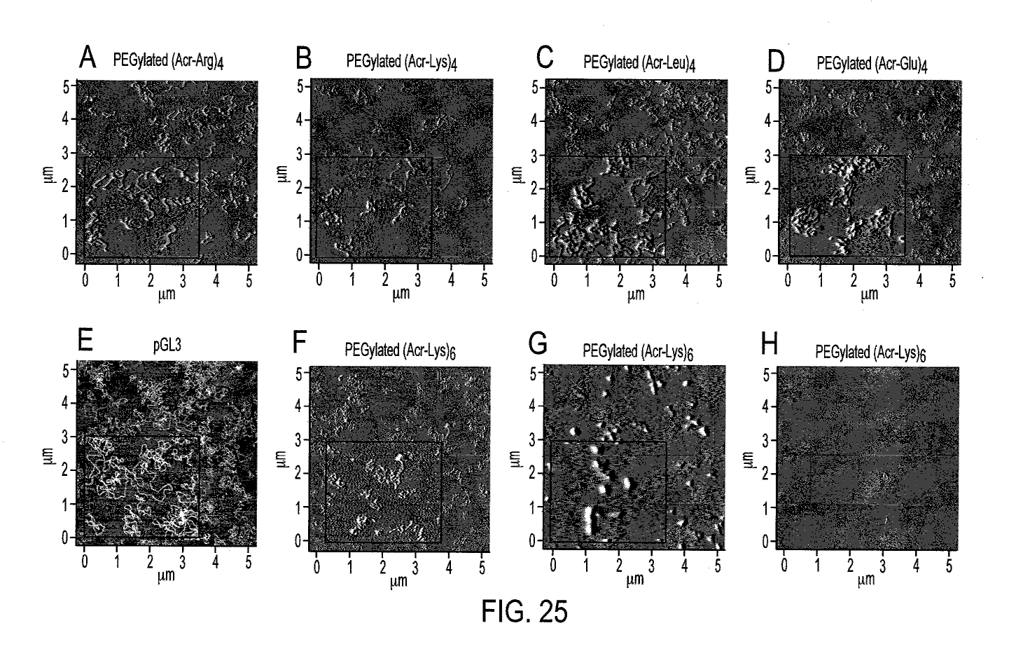

The shape and relative charge of PEGylated polyacridine polyplexes was further examined by atomic force microscopy (AFM) (FIGS. 25A-25H). Polyplexes prepared at 0.8 nmol per μg of DNA using PEGylated (Acr-Arg)₄, (Acr-Lys)₄, (Acr-Leu)₄ and (Acr-Glu)₄ (SEQ ID NOS 28-31, respectively), each produced anionic open-polyplexes that bound to cationically charged mica (FIGS. 25A-D). The relative size and shape appeared as open-polyplexes of approximately 0.2-0.3 μm, relative to naked plasmid DNA which also bound to cationic mica to produce a more open coiled structure of approximately 0.5 μm diameter (FIG. 25E). Examination of AFM images of PEGylated (Acr-Lys)₆ polyplexes (SEQ ID NO: 33) revealed anionic open-polyplexes that bound to cationic mica at 0.2 nmol per μg of DNA (FIG. 25F). Alternatively, 0.8 nmol per μg of DNA produced cationic closed-polyplexes of slightly smaller size that bound to anionic mica (FIG. 25G). Cationic closed-polyplexes were only observed on anionic mica, whereas an attempt to achieve binding to cationic mica resulted in images devoid of visible polyplexes (FIG. 25H). These results illustrate the unique shape of anionic open-polyplexes as well as the ability to manipulate the charge of PEGylated (Acr-Lys)₆ polyplexes (SEQ ID NO: 33) to be either cationic or anionic and change shape from open to closed-polyplexes depending on peptide stoichiometry.

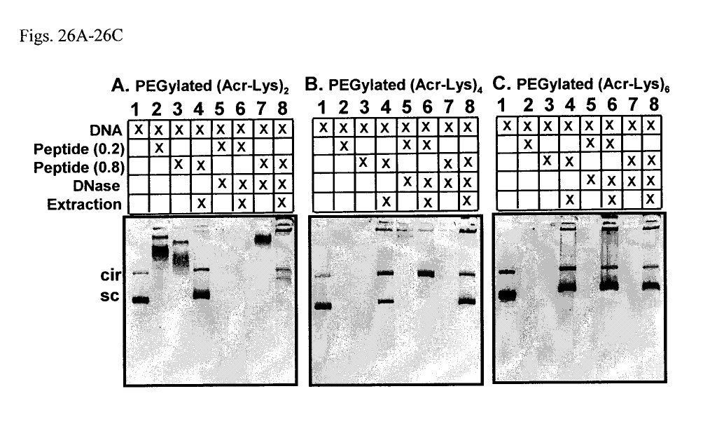

The unique shape and charge of anionic PEGylated open-polyplexes should minimize binding to serum proteins and extend the pharmacokinetic half-life of plasmid DNA, provided that the DNA remains protected from premature metabolism. To investigate the metabolic stability of DNA polyplexes, PEGylated (Acr-Lys)_{2, 4, and 6} polyplexes (SEQ ID NOS 32, 29, and 33, respectively) were digested with DNase, followed by phenol-chloroform extraction to remove peptide and gel electrophoresis to determine the status of the plasmid DNA (FIGS. 26A-26C). Unique band shifts occurred for PEGylated (Acr-Lys)_{2, 4, and 6} polyplexes (SEQ ID NOS 32, 29, and 33, respectively) prepared at either 0.2 or 0.8 nmols of peptide per μg of plasmid DNA (FIG. 26, lane 2 and 3 of panels 26A, 26B, 26C). However, phenol-chloroform extraction removed the PEG-peptide allowing recovery of the DNA which migrated coincident with control (FIG. 26, lane 4 of panels 26A, 26B and 26C). When challenged with a DNase digestion, PEGylated (Acr-Lys)_{2 or 4} polyplexes (SEQ ID NOS 32 and 29, respectively) prepared at 0.2 nmol per μg of DNA failed to protect DNA from metabolism (FIG. 26, panel 26A and 26B, lane 5 and 6). In contrast, polyplexes formed at 0.2 nmols PEGylated (Acr-Lys)₆ (SEQ ID NO: 33), protected the DNA from DNase resulting in the recovery of bands following extraction (FIG. 26C, lanes 5 and 6). At a higher stoichiometry of 0.8 nmols, PEGylated (Acr-Lys)_{2, 4 and 6} (SEQ ID NOS 32, 29, and 33, respectively) each protected DNA from metabolism (FIG. 26, panel A-C, lanes 7 and 8). These results establish a clear correlation between polyacridine DNA binding affinity (FIGS. 23A and 23B) and metabolic stability of polyplexes. Most importantly, PEGylated (Acr-Lys)₆ (SEQ ID NO: 33) is able to form DNase stable anionic open-polyplexes when prepared at 0.2, as well as cationic closed-polyplexes prepared at 0.8 nmol per μg of DNA.

PEGylated polyacridine peptides were also subjected to trypsin digestion to determine if they could be digested by a common serine protease, suggesting perhaps they could also be more easily cleared from cells. The results established that even as little as 100 mU of trypsin catalyzed the formation of a dipeptide of Lys-Lys(Acr) with mass of 451 g/mol, to a reaction completion within 1 hr. Thereby, the high-affinity DNA binding of PEGylated polyacridine peptides is greatly reduced upon proteolytic digestion.

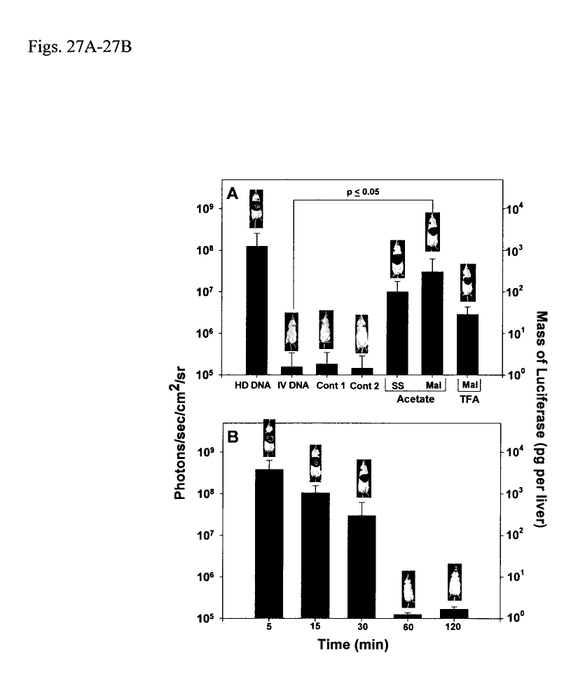

To determine if polyacridine peptides would mediate gene transfer, we chose to examine the ability of PEGylated DNA polyplexes to produce luciferase expression in the liver of mice, 24 hrs following a 1 μg dose of pGL3. Hydrodynamic dosing of 1 μg of pGL3 produced 10⁸ photon/sec/cm²/sr determined using a calibrated bioluminescence assay (FIG. 27A, HD DNA). When pGL3 (1 μg in 50 μl) was dosed via the tail vein without hydrodynamic delivery, there was no detectable luciferase expression (not shown). In an attempt to stimulate gene expression, PEGylated polyacridine peptide polyplexes were administered via the tail vein (1 μg of pGL3 in 50 μl) and after 30 min a hydrodynamic dose (2 ml) of saline was used to stimulate liver uptake and gene expression (FIG. 27A). In the subsequent studies described below, stimulated expression produced 10⁷-10⁹ photon/sec/cm²/sr, the magnitude being dependent on the structure of the PEGylated polyacridine peptide, the time delay between primary dose and stimulation dose, the charge ratio at which the polyplex was prepared, and the amount of DNA dosed.

At a fixed dose of 1 μg of pGL3 and a time delay of 30 min between primary and stimulatory dose, polyplexes prepared at 0.5 nmol of PEGylated (Acr-Arg)₄ (SEQ ID NO: 28), in which the PEG was attached by a maleimide (Mal) or a reducible disulfide (SS) linkage, were administered to mice and analyzed by BLI after 24 hrs. Approximately 5-fold higher luciferase expression was observed for Mal versus SS, indicating that a reducible linkage offered no advantage for stimulated expression (FIG. 27A). Comparison of the Mal peptide prepared as either an acetate or TFA salt form (ion paired with Arg) resulted in an approximately 10-fold loss of gene transfer for the TFA form (FIG. 27A). These results justified the use of Mal peptides in the acetate salt form for further comparisons.

Several controls were applied to further establish that PEGylated polyacridine peptides were necessary to achieve stimulated expression. The administration of a 1 μg dose of pGL3 followed by a stimulatory dose of saline after 30 min resulted in no detectable luciferase expression at 24 hrs (FIG. 27A, IV DNA). Likewise, there was no detectable luciferase expression with a 1 μg dose of pGL3 prepared as a PEGylated-Cys-Trp-Lys₁₈ polyplex (SEQ ID NO: 34) (FIG. 27A, cont 1), or PEGylated glycoprotein polyplex⁴¹ (FIG. 27A, cont 2). These controls confirmed that naked DNA was not responsible for the observed expression and also established that not all gene formulations were capable of mediating stimulated expression.

The stimulated expression mediated by PEGylated (Acr-Arg)₄ polyplexes (SEQ ID NO: 28) (1 μg) was examined to determine the influence of the delay time between primary and stimulatory dose. Varying the delay from 5-120 min established a 10-fold loss in luciferase expression between 5 and 30 min, followed by a complete loss of stimulated expression at 60 min and longer (FIG. 27B). Interestingly, the magnitude of stimulated gene expression at a delay time of 5 min was approximately 5-fold greater than direct hydrodynamic dosing of 1 μg of pGL3 (FIGS. 27A and B).

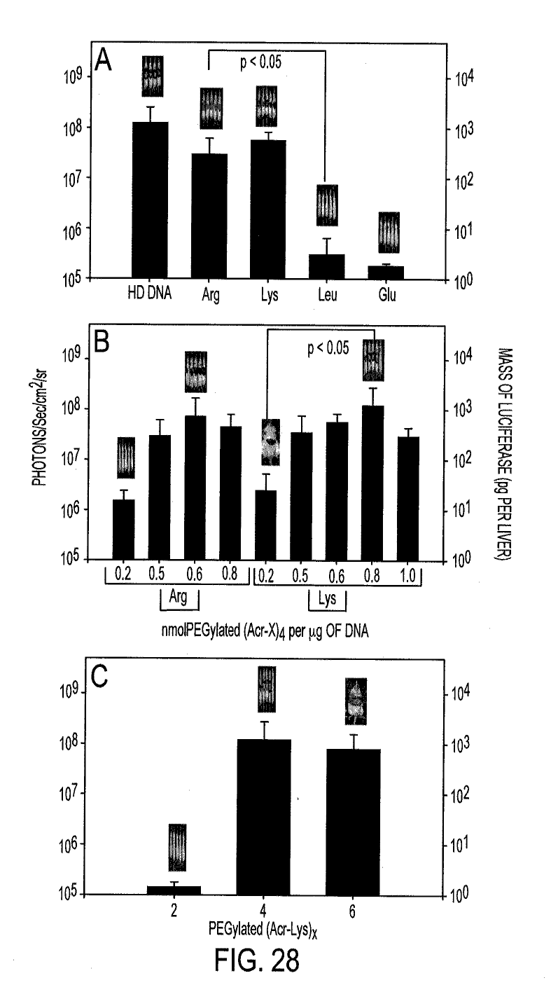

To determine how the spacing amino acid influence stimulated gene expression, PEGylated peptides of general structure (Acr-X)₄, possessing X as either Lys, Arg, Leu or Glu (SEQ ID NOS 29, 28, 30, and 31, respectively) were used to prepare polyplexes that were dosed in mice and stimulated to express luciferase. While the DNA dose was fixed at 1 μg, and the time delay was 30 min, the stoichiometry of PEGylated peptide to DNA was adjusted based on the results of zeta potential titration (FIG. 24) to favor complete polyplex formation. The results established that only PEGylated peptides possessing an Arg or Lys spacing amino acid produced appreciable levels of luciferase expression (FIG. 28A), whereas peptides with Glu and Leu spacers were essentially inactive.

The stoichiometry of both Lys and Arg spaced PEGylated polyacridine peptide were varied from 0.2-1 nmol of peptide per μg of DNA, then dosed via tail vein and stimulated after 30 min. A 1 μg open polyplex prepared with 0.6 nmol of PEGylated (Acr-Arg)₄ (SEQ ID NO: 28) or 0.8 nmol of PEGylated (Acr-Lys)₄ (SEQ ID NO: 29) mediated maximal luciferase expression when assayed by BLI at 24 hrs (FIG. 28B). Both the Lys and Arg spaced PEGylated peptide mediated approximately 100-fold lower gene expression at 0.2 nmols of peptide compared to the stoichiometry of maximal expression (FIG. 28B). Likewise, at stoichiometries greater than maximal expression, a decline in expression was observed. These results suggested that a Lys spacer may have a slight advantage over Arg, which then prompted an investigation into varying the number of (Acr-Lys) repeats.

Comparison of the stimulated gene expression (30 min) mediated by

PEGylated (Acr-Lys)_{2, 4 and 6} polyplexes (SEQ ID NOS 32, 29, and 33, respectively) (1 μg pGL3) prepared at an optimized stoichiometry of 1, 0.8 and 0.2 nmols of peptide respectfully (based on zeta potential, FIG. 24), established that PEGylated (Acr-Lys)₂ (SEQ ID NO: 32) was completely inactive. In contrast, PEGylated (Acr-Lys)₆ (SEQ ID NO: 33) at 0.2 nmols matched the activity of PEGylated (Acr-Lys)₄ (SEQ ID NO: 29) at 0.8 nmols (FIG. 28C). These results correlate with PEGylated (Acr-Lys)₆ (SEQ ID NO: 33) having a higher binding affinity for DNA (FIG. 23), with a greater ability to protect DNA from metabolism (FIG. 24), and with the ability to mediate stimulated gene expression even at a low stoichiometry of 0.2 nmols per μg of DNA (FIG. 28).

When PEGylated (Acr-Lys)₆ polyplexes (SEQ ID NO: 33) were administered to mice to determine how changing stoichiometry influenced gene expression, the results established that a 1 μg dose of pGL3 polyplex, stimulated at 30 min, produced equivalent gene expression across the range of 0.2-0.8 nmols of peptide (FIG. 28A). These results are in sharp contrast to those determined for PEGylated (Acr-Lys)₄ (SEQ ID NO: 29) (FIG. 28B), in which the gene expression was highly dependent on the stoichiometry of peptide to DNA. Furthermore, since PEGylated (Acr-Lys)₆ (SEQ ID NO: 33) was the only peptide to produce a zeta potential titration that started anionic (−5 mV) at 0.2 nmol and titrated to cationic (+5 mV) at 0.6 nmols (FIG. 24F), it appeared that the stimulated gene expression mediated by PEGylated (Acr-Lys)₆ (SEQ ID NO: 33) is fully functional as both an anionic open polyplex (FIG. 25F), and as an cationic closed polyplex (FIG. 25G).

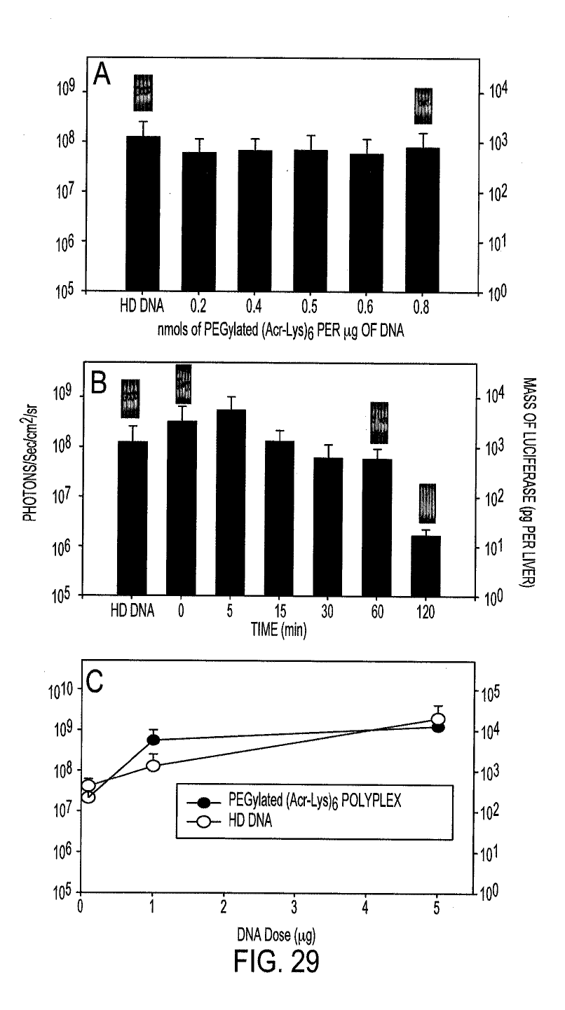

To determine if the unique DNA binding properties of PEGylated (Acr-Lys)₆ (SEQ ID NO: 33) would also extend the delay time allowed between primary and stimulatory dose, polyplexes (1 μg) were prepared with 0.2 nmols of peptide and administered with a time delay varying from 0 to 120 min. Delaying the stimulation from 5 to 60 min resulted in only a slight 2-3 fold decrease in expression, whereas with a 120 min delay the expression decreased 100-fold (FIG. 29B). Using a zero delay time resulted in a nearly 5-fold increase in gene expression relative to dose equivalent direct HD administration of pGL3 (FIG. 29B). These results demonstrate that PEGylated (Acr-Lys)₆ (SEQ ID NO: 33) mediates significant high-level gene expression, even with a 60 min delay time, compared to PEGylated (Acr-Arg)₄ (SEQ ID NO: 28) which failed to mediate detectable expression following a 60 min delay in stimulation (FIG. 27B).

To establish the dose-equivalency of direct HD administration of DNA, relative to the stimulated expression of PEGylated polyacridine polyplexes, a dose-response experiment was performed (FIG. 29C). The BLI detected expression at 24 hrs post-DNA delivery was determined for direct HD delivered pGL3, dosed at 0.1, 1 and 5 μg of DNA (FIG. 29C). This was compared with the 24 hr luciferase expression from identical DNA doses of PEGylated (Acr-Lys)₆ polyplexes (SEQ ID NO: 33) prepared with 0.2 nmol of peptide and administered with a 5 min delay in stimulation. A nearly linear dose-response curve was identified for direct HD dosing of pGL3, whereas PEGylated (Acr-Lys)₆ polyplexes (SEQ ID NO: 33) showed an increase from 0.1-1 μg, followed by a plateau at 5 μg dose (FIG. 29C). At a dose of 1 μg of DNA, PEGylated (Acr-Lys)₆ (SEQ ID NO: 33) mediated approximately 5-fold greater gene expression (FIG. 29C).

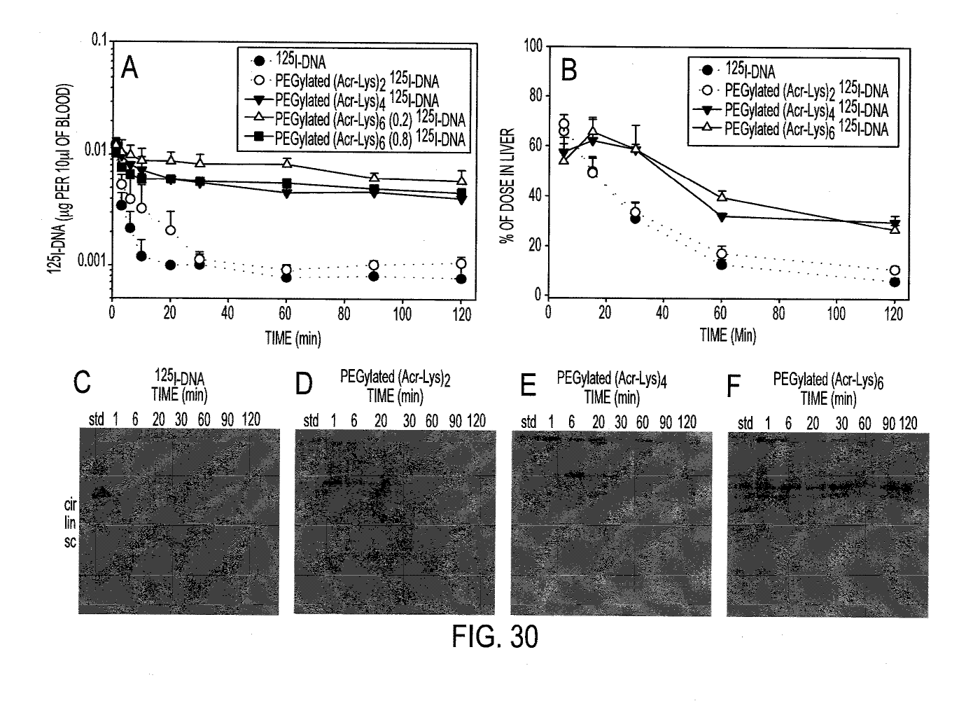

To gain further mechanistic insight into the reason that PEGylated polyacridine polyplexes mediate stimulated gene expression, pharmacokinetic and biodistribution studies were performed. Following i.v. dosing of ¹²⁵I-DNA or PEGylated (Acr-Lys)₂ polyplexes (SEQ ID NO: 32) resulted in a rapid 10-fold loss of radioactivity from the blood within 20-30 min, followed by an apparent long half-life (FIG. 30A). Isolation of ¹²⁵I-DNA from blood time points, followed by gel electrophoresis and autoradiography demonstrated that plasmid DNA was rapidly degraded to fragments within 1 min (FIG. 30C). Likewise, electrophoretic analysis of blood time points following dosing of PEGylated (Acr-Lys)₂ ¹²⁵-DNA polyplexes (SEQ ID NO: 32) demonstrated complete loss of plasmid DNA within 20 min (FIG. 30D), suggesting that the modest binding from PEGylated (Acr-Lys)₂ (SEQ ID NO: 32) leads to slightly delayed metabolism.

The pharmacokinetic profile for PEGylated (Acr-Lys)_{4 and 6} ¹²⁵I-DNA polyplexes (SEQ ID NOS 29 and 33, respectively) were both very similar and distinct from those of ¹²⁵I-DNA and PEGylated (Acr-Lys)₂ ¹²⁵I-DNA polyplexes (SEQ ID NO: 32) (FIG. 30A). The radioactivity in blood only decreased 3-4 fold in the first 20 min and proceeded with an apparent long half-life (FIG. 30A). Recovery of ¹²⁵I-DNA from PEGylated (Acr-Lys)_{4 and 6} polyplexes (SEQ ID NOS 29 and 33, respectively) resulted in an agarose gel autoradiographic analysis that established the blood stability of plasmid DNA up to 30 min for PEGylated (Acr-Lys)₄ (SEQ ID NO: 29) (FIG. 30E), and up to 120 min for PEGylated (Acr-Lys)₆ (SEQ ID NO: 33) (FIG. 30F). Non-linear least squares analysis of the radioactivity in blood over time was used to calculate an apparent α-half-life of 2-3 min and a β-half-life of 65 min for PEGylated (Acr-Lys)₄ (SEQ ID NO: 29) and 120 min for PEGylated (Acr-Lys)₆ (SEQ ID NO: 33) (Table 5).

TABLE 5
Pharmacokinetic Parameters for PEGylated Polyacridine Polyplexes

| Polyacridine Peptide | t_{1/2}α^{a} | t_{1/2}β^{b} | Vol Dis^{c} | CL^{d} | MRT^{e} | AUC^{f} |
|---|---|---|---|---|---|---|
| Polyplex | (min) | (min) | (ml) | (ml/min) | (min) | (μa*min)/ml |
| PEGylated (Acr-Lys)₂ | 0.7 +/- 0.0 | 15.2 +/- 0.8 | 42.8 +/- 0.1 | 2.3 +/- 0.0 | 18.9 +/- 0.1 | 0.1 +/- 0.0 |
| ¹²⁵I-DNA^{g} (SEQ ID | | | | | | |
| NO: 32) | | | | | | |
| PEGylated (Acr-Lys)₄ | 2.3 +/- 0.3 | 65.6 +/- 13.5 | 37.4 +/- 1.9 | 0.4 +/- 0.1 | 92.2 +/- 18.9 | 0.7 +/- 0.1 |
| ¹²⁵I-DNA^{h} (SEQ ID | | | | | | |
| NO: 29) | | | | | | |
| PEGylated (Acr-Lys)₆ | 1.8 +/- 0.9 | 181.5 +/- 33.4 | 31.6 +/- 1.3 | 0.1 +/- 0.0 | 260.8 +/- 47.8 | 2.5 +/- 0.4 |
| ¹²⁵I-DNAⁱ (SEQ ID | | | | | | |
| NO: 33) | | | | | | |

^{a}Calculated α-half-life.
^{b}Calculated β-half-life.
^{c}Volume of distribution.
^{d}Total body clearance rate.
^{e}Mean residence time.
^{f}Area under the curve.
^{g}Calculated using blood cpm values over 20 min, assuming complete DNA stability.
^{h}Calculated using blood cpm values over 60 min, assuming complete DNA stability.
ⁱCalculated using blood cpm values over 120 min, assuming complete DNA stability.

For the purpose of comparison, complete metabolic stability of plasmid DNA was assumed in the pharmacokinetic calculation, even though the gel electrophoretic and autoradiographic analysis for PEGylated (Acr-Lys)₂ and (Acr-Lys)₄ (SEQ ID NOS 32 and 29, respectively) establish the differential loss of intact DNA over time. The apparent clearance rate decrease and the mean residence time increased as the β-half-live increased, whereas the apparent volume of distribution remained nearly constant. This resulted in an increase in the AUC as the stability of the polyplex increased (Table 5).

The unique long pharmacokinetic β-half-life for PEGylated (Acr-Lys)_{4 and 6} polyplexes (SEQ ID NOS 29 and 33, respectively) and the nearly coincident volume of distribution suggested there would be similar biodistribution to the organs. The experimental result established that the liver was the major site of biodistribution for PEGylated (Acr-Lys)_{2, 4 and 6} ¹²⁵I-DNA polyplexes (SEQ ID NOS 32, 29, and 33, respectively) (Table 6). In each of these experiments, it was not possible to extract ¹²⁵I-DNA from liver or other tissues to determine its metabolic status, as was performed for DNA in blood.

TABLE 6
Biodistribution Parameters for PEGylated Polyacridine Polyplexes

| Polyacridine | | | | | | | | | | | |
|---|---|---|---|---|---|---|---|---|---|---|---|
| Peptide | Time | | | | | | | | | | |
| Polyplex | (min) | Blood^{a} | Liver^{b} | Lung^{b} | Spleen^{b} | Stomach^{b} | Kidney^{b} | Heart^{b} | LI^{b} | SI^{b} | Total^{c} |
| ¹²⁵I-DNA | 5 | 14.4 ± 5.8 | 65.7 ± 2.5 | 6.1 ± 2.2 | 2.8 ± 0.3 | 0.2 ± 0.0 | 0.9 ± 0.2 | 0.1 ± 0.0 | 0.2 ± 0.1 | 0.4 ± 0.1 | 84.7 ± 11.2 |
| | 30 | 6.8 ± 1.7 | 31.1 ± 6.2 | 1.4 ± 0.1 | 2.1 ± 0.6 | 1.6 ± 1.3 | 3.5 ± 1.0 | 0.2 ± 0.0 | 1.6 ± 0.7 | 2.8 ± 0.3 | 51.1 ± 11.9 |
| | 60 | 5.2 ± 1.6 | 12.7 ± 2.0 | 0.9 ± 0.2 | 1.3 ± 0.2 | 4.5 ± 1.0 | 3.5 ± 0.6 | 0.2 ± 0.0 | 1.1 ± 0.2 | 2.1 ± 0.3 | 31.5 ± 6.2 |
| | 120 | 5.2 ± 1.5 | 6.1 ± 1.5 | 0.5 ± 0.2 | 0.8 ± 0.2 | 9.4 ± 3.2 | 1.6 ± 0.9 | 0.1 ± 0.1 | 1.9 ± 0.8 | 2.6 ± 0.5 | 28.2 ± 8.9 |
| PEGylated | 5 | 26.4 ± 14.0 | 68.9 ± 3.6 | 1.9 ± 0.1 | 5.3 ± 1.8 | 0.2 ± 0.0 | 0.8 ± 0.5 | 0.1 ± 0.1 | 0.3 ± 0.2 | 0.4 ± 0.1 | 104.2 ± 20.5 |
| (Acr-Lys)₂ | 30 | 7.6 ± 1.3 | 33.6 ± 4.0 | 1.0 ± 0.3 | 1.7 ± 0.4 | 1.5 ± 0.3 | 2.8 ± 0.6 | 0.2 ± 0.1 | 1.9 ± 0.5 | 2.5 ± 0.5 | 52.8 ± 8.1 |
| ¹²⁵I-DNA | 60 | 6.2 ± 0.6 | 17.1 ± 3.1 | 0.7 ± 0.2 | 0.7 ± 0.5 | 6.1 ± 2.0 | 2.6 ± 1.1 | 0.2 ± 0.1 | 2.3 ± 0.5 | 3.1 ± 1.1 | 39.1 ± 9.3 |
| (SEQ ID | 120 | 7.2 ± 1.0 | 10.9 ± 1.5 | 0.8 ± 0.2 | 1.0 ± 0.4 | 8.3 ± 1.7 | 2.3 ± 0.4 | 0.2 ± 0.0 | 1.9 ± 0.4 | 3.6 ± 0.7 | 36.2 ± 6.4 |
| NO: 32) | | | | | | | | | | | |
| PEGylated | 5 | 54.1 ± 2.7 | 57.5 ± 5.8 | 2.2 ± 0.9 | 2.1 ± 0.5 | 0.3 ± 0.2 | 0.9 ± 0.5 | 0.2 ± 0.1 | 0.3 ± 0.2 | 0.5 ± 0.2 | 118.0 ± 11.1 |
| (Acr-Lys)₄ | 30 | | 58.6 ± 9.7 | 1.0 ± 0.1 | 4.5 ± 2.3 | 0.5 ± 0.1 | 1.4 ± 0.9 | 0.1 ± 0.1 | 0.5 ± 0.1 | 0.9 ± 0.5 | 104.8 ± 18.3 |
| ¹²⁵I-DNA | 60 | 37.3 ± 4.4 | 32.2 ± 1.2 | 0.8 ± 0.3 | 4.7 ± 0.3 | 2.6 ± 0.4 | 2.1 ± 0.8 | 0.2 ± 0.0 | 1.1 ± 0.2 | 2.7 ± 0.7 | 77.6 ± 6.1 |
| (SEQ ID | 120 | 31.2 ± 2.2 | 29.9 ± 2.7 | 1.0 ± 0.2 | 4.1 ± 2.4 | 5.3 ± 2.4 | 2.7 ± 1.5 | 0.2 ± 0.1 | 1.5 ± 0.6 | 1.7 ± 0.1 | 73.9 ± 13.3 |
| NO: 29) | | 27.4 ± 3.2 | | | | | | | | | |
| PEGylated | 5 | 65.8 ± 14.3 | 53.8 ± 7.0 | 1.3 ± 0.5 | 6.8 ± 3.1 | 0.4 ± 0.2 | 0.7 ± 0.1 | 0.2 ± 0.0 | 0.2 ± 0.1 | 0.5 ± 0.0 | 129.6 ± 25.3 |
| (Acr-Lys)₆ | 30 | 54.9 ± 12.3 | 58.7 ± 2.0 | 0.9 ± 0.2 | 11.7 ± 1.1 | 0.7 ± 0.2 | 1.1 ± 0.1 | 0.2 ± 0.1 | 0.5 ± 0.3 | 0.9 ± 0.2 | 129.6 ± 16.5 |
| ¹²⁵I-DNA | 60 | 55.2 ± 7.6 | 39.6 ± 2.8 | 0.6 ± 0.1 | 15.4 ± 0.1 | 1.3 ± 0.3 | 1.3 ± 0.4 | 0.1 ± 0.1 | 0.8 ± 0.3 | 1.5 ± 0.2 | 115.9 ± 11.8 |
| (SEQ ID | 120 | 39.4 ± 10.2 | 26.8 ± 2.1 | 0.8 ± 0.2 | 18.0 ± 1.4 | 3.2 ± 2.8 | 1.5 ± 0.2 | 0.1 ± 0.0 | 1.1 ± 0.7 | 2.2 ± 0.3 | 93.2 ± 17.9 |
| NO: 33) | | | | | | | | | | | |

^{a}Percent of dose based on pharmacokinetic analysis.
^{b}Percent of dose based on gamma counting of tissue.
^{c}Total percent of dose recovered.

The biodistribution of ¹²⁵I-DNA and PEGylated (Acr-Lys)₂ ¹²⁵I-DNA polyplexes (SEQ ID NO: 32) both reached a maximum of approximately 65% in the liver at 5 min, followed by a decrease to less than 10% by 120 min (FIG. 30B). In contrast, when dosing PEGylated (Acr-Lys)_{4 and 6} ¹²⁵I-DNA polyplexes (SEQ ID NOS 29 and 33, respectively) the percent of radioactive dose in liver was approximately 55% at 5 min, which accumulated to over 60% at 15 min, followed by a decline to 30-40% at 120 min (FIG. 30B). By comparison, a maximum of 1-2% of the total radioactive dose distributed to lungs, heart, kidneys and intestine at all time points (Table 6). The exception was the spleen which showed a steady accumulation over time reaching 18% at 2 hrs when dosing PEGylated (Acr-Lys)₆ polyplexes (SEQ ID NO: 33). In addition to the liver and spleen, the blood accounted for the majority of the radioactive dose, which ranged from 65-40% in the circulation during 2 hrs following dosing of PEGylated (Acr-Lys)₆ polyplexes (SEQ ID NO: 33).

Discussion

PEGylated polyacridine peptides were designed to bind to plasmid DNA for the purpose of making it more blood compatible as a first step toward ultimately adding a targeting ligand and sub-cellular targeting peptides, needed to complete the delivery system and achieve significant in vivo gene expression following i.v. dosing, without the requirement of an additional stimulation. To accomplish this goal, an optimal polyacridine peptide would need to bind to DNA with sufficient affinity to protect from DNase metabolism in the circulation, while being able to release the DNA inside the cell to gain access to the nucleus.

It is likely there are many unique sequences of polyacridine peptides that accomplish this goal. The aim of the structure-activity study described was to determine a relationship between the number of Acr, the binding affinity for DNA, the polyplex pharmacokinetic half-life, and the magnitude of stimulated expression in mice. Initially, a nine amino acid peptide allowed the incorporation of four Acr spaced by a hydrophobic (Leu), anionic (Glu), or cationic (Lys and Arg) residue, along with a C-terminal Cys for modification. Prior studies from our group established that spacing amino acids that lacked a bulky side-chain or protecting group (Gly or Ala) resulted in low peptide yields. During the course of this study it was necessary to further optimize peptide yield to prepare (Acr-Lys)₆-Cys (SEQ ID NO: 92). While it is possible to prepare even longer polyacridine peptides of this design, we found the yields began to diminish. Each of the polyacridine peptides was coupled to PEG_{5000Da}, resulting in PEGylated polyacridine peptide that produced a single symmetrical peak on RP-HPLC and a MALDI-TOF MS that verified the structure.

The primary structural features that influence the level of gene expression mediated by PEGylated polyacridine peptides was the presence of at least four Acr residues combined with cationic (Lys or Arg) spacing amino acid. PEGylated polyacridine peptides possessing four Acr residues spaced by either Glu or Leu demonstrated weak binding to DNA (FIG. 23A), resulting in no stimulated gene expression (FIG. 28A), presumably due to the inability to protect DNA from metabolism in vivo. Conversely, PEGylated (Acr-Lys)₄ or (Acr-Arg)₄ (SEQ ID NOS 29 and 28, respectively) possessed higher affinity for binding with DNA (FIG. 23A), which correlated with their ability to produce stimulated expression (FIG. 28A), due to their ability to stabilize DNA from premature metabolism in vivo (FIG. 30E). We previously reported that PEGylated Cys-Trp-Lys₁₈ (SEQ ID NO: 34) formed polyplexes that readily dissociate in the blood, resulting in rapid DNA metabolism (Collard W T et al. Biodistribution, metabolism, and in vivo gene expression of low molecular weight glycopeptide polyethylene glycol peptide DNA co-condensates. Journal of Pharmaceutical Sciences 2000; 89: 499-512). It was therefore anticipated that stimulation of i.v. dosed PEGylated Cys-Trp-Lys₁₈ polyplexes (SEQ ID NO: 34) would not produce gene expression (FIG. 27A, cont 1). Thus, a combination of polyintercalation and ionic binding to plasmid DNA results in improved affinity and stable polyplexes, where either functionality alone is insufficient.

One of the more unique aspects of PEGylated polyacridine polyplexes is their open polyplex structures that resemble naked DNA (FIG. 25). PEGylated (Acr-Lys)₄ and (Acr-Arg)₄ (SEQ ID NOS 29 and 28, respectively) produced anionic open-polyplexes across the titration range of 0.2-0.8 nmols of peptide (FIGS. 24A and E). When i.v. dosed and allowed to circulate for 30 min, the level of stimulated expression was dependent upon the stoichiometry of peptide to DNA, resulting in a parabolic peptide dose-response curve (FIG. 28B). These results suggested that the DNA stability afforded by PEGylated (Acr-Lys)₄ and (Acr-Arg)₄ (SEQ ID NOS 29 and 28, respectively), was dependent on peptide stoichiometry, with higher stoichiometries resulting in charge neutral polyplexes (FIG. 24A) that stabilize DNA from metabolism (FIG. 26B) and demonstrate diminished gene transfer efficiency (FIG. 28B).

PEGylated (Acr-Lys)₆ (SEQ ID NO: 33) binds to DNA with higher affinity compared to all other peptides studied (FIG. 23). It also possessed an unusual zeta potential titration curve that produced anionic open-polyplexes at 0.2 nmols of peptide, which convert to cationic closed-polyplexes at 0.8 nmols and higher (FIG. 24F and FIGS. 25F & G). The DNase stability afforded to PEGylated (Acr-Lys)₆ polyplexes (SEQ ID NO: 33) at both 0.2 and 0.8 nmols of peptide (FIG. 26C) resulted in equivalent, high-level stimulated gene expression at each stoichiometry from 0.2-0.8 nmols (FIG. 29A). The greater affinity also led to greater stimulated expression for longer delay times compared to PEGylated (Acr-Arg)₄ polyplexes (SEQ ID NO: 28), such that following the administration of 1 μg of DNA, a 1 hour delayed stimulation produced high-level gene expression, and at 2 hour delay measurable expression was still detected (FIG. 29B). These results established that PEGylated (Acr-Lys)₆ polyplexes (SEQ ID NO: 33) mediated equal gene expression as either closed or open-polyplex structures, and suggest that enhanced binding affinity translates to higher expression at longer delay times due to postponing metabolism.

Pharmacokinetic and biodistribution studies were used to gain insight into the underlying mechanism of how delayed hydrodynamic stimulation caused PEGylated (Acr-Lys)₆ polyplexes (SEQ ID NO: 33) to mediate gene expression in the liver. The most striking result was the apparent long pharmacokinetic half-life of PEGylated (Acr-Lys)₄ (SEQ ID NO: 29) and PEGylated (Acr-Lys)₆ polyplexes (SEQ ID NO: 33), compared to the rapid loss of ¹²⁵I-DNA and PEGylated (Acr-Lys)₂ polyplexes (SEQ ID NO: 32) (FIG. 30A). Electrophoretic analysis of the ¹²⁵I-DNA recovered from blood clearly established that PEGylated (Acr-Lys)₆ (SEQ ID NO: 33) stabilized DNA in the circulation for 2 hrs (FIG. 30F), whereas PEGylated (Acr-Lys)₄ (SEQ ID NO: 29) stabilized open circular DNA bands for at least 30 min (FIG. 30E), and PEGylated (Acr-Lys)₂ (SEQ ID NO: 32) for no more than 20 min (FIG. 30D). Most importantly, approximately 40% of the radioactive dose remained in the blood after 2 hrs when dosing PEGylated (Acr-Lys)₆¹²⁵I-DNA polyplexes (SEQ ID NO: 33) (Table 6), and the pharmacokinetic profile appears to be the same whether dosing anionic-open or cationic-closed polyplexes (FIG. 30A).

These results cannot be directly compared with several prior studies that reported long circulating DNA or siRNA formulations by analyzing the pharmacokinetics of polyplexes by incorporating a fluorophore radiolabel into the carrier component (Heyes J et al. Lipid encapsulation enables the effective systemic delivery of polyplex plasmid DNA. Mol Ther 2007; 15: 713-720; Kunath K et al. The Structure of PEG-Modified Poly(Ethylene Imines) Influences Biodistribution and Pharmacokinetics of Their Complexes with NF-κB Decoy in Mice. Pharmaceutical Research 2002; 19: 810-817; Li S-D, Chen Y-C, Hackett M J, Huang L. Tumor-targeted Delivery of siRNA by Self-assembled Nanoparticles. Mol Ther 2007; 16: 163-169; Morille M et al. Long-circulating DNA lipid nanocapsules as new vector for passive tumor targeting. Biomaterials; 31: 321-329). These prior studies are more closely related in their direct analysis of pharmacokinetics using radiolabeled DNA^{45,46} or by quantifying DNA in blood and tissues by PCR (Zhou Q-H, Wu C, Manickam D, Oupicý D. Evaluation of Pharmacokinetics of Bioreducible Gene Delivery Vectors by Real-time PCR. Pharmaceutical Research 2009; 26: 1581-1589). These reports establish the ability to extend the half-life of DNA in the circulation, but stop short of establishing the metabolic status of recovered DNA. This is the first study to demonstrate stabilized plasmid DNA circulating in mice by gel electrophoresis and autoradiography. The metabolic stability of DNA is directly related to the mode and affinity of binding achieved by PEGylated (Acr-Lys)₆ (SEQ ID NO: 33). A similar PEGylated polylysine peptide (PEG-Cys-Trp-Lys₁₈ (SEQ ID NO: 34)) also binds with high affinity to DNA through ionic interaction and forms cationic-closed polyplexes (Kwok K Y, McKenzie D L, Evers D L, Rice K G. Formulation of highly soluble poly(ethylene glycol)-peptide DNA condensates. Journal of Pharmaceutical Sciences 1999; 88: 996-1003). However, despite being a longer peptide than PEGylated (Acr-Lys)₆ (SEQ ID NO: 33), PEG-Cys-Trp-Lys₁₈ polyplexes (SEQ ID NO: 34) rapidly dissociate following i.v. dosing leading to the complete metabolism of plasmid DNA in the circulation within 3 min (Collard W T et al. Biodistribution, metabolism, and in vivo gene expression of low molecular weight glycopeptide polyethylene glycol peptide DNA co-condensates. Journal of Pharmaceutical Sciences 2000; 89: 499-512). Thereby, it is the combination of polyintercalation and cationic binding that appears to be essential to achieve DNA stability during circulation, whereas PEGylated peptides that bind DNA by either mode individually are insufficient. This unique feature suggests that PEGylated (Acr-Lys)₆ polyplexes (SEQ ID NO: 33) may find application in targeting DNA to tissues outside the liver.

Analysis of the tissue distribution of ¹²⁵I-DNA polyplexes over time established the liver as the major site of distribution accounting for approximately 54-69% of the dose within the first 5 min, with only minor (<1%) distribution to other organs. PEGylated (Acr-Lys)₄ (SEQ ID NO: 29) and PEGylated (Acr-Lys)₆ polyplexes (SEQ ID NO: 33) produced a distinct liver distribution and metabolism profile, with maximal accumulation of 60% at 20 min followed by a decrease to 30% over two hrs (FIG. 30A). The liver biodistribution profile of ¹²⁵I-DNA and PEGylated (Acr-Lys)₂ ¹²⁵I-DNA polyplexes (SEQ ID NO: 32) were coincident with maximal accumulation of 65% at 5 min, followed by a decrease to 10% over 2 hrs. The stability of plasmid DNA in the blood afforded by PEGylated (Acr-Lys)₆ (SEQ ID NO: 33) appears to extrapolate to similar stability in the liver. Taken together, the pharmacokinetic and biodistribution data support a hypothesis in which the DNA that distributes to the liver is primarily responsible for the stimulated expression. However, liver distribution is not sufficient, the DNA must also be sufficiently metabolically stabilized to mediate significant stimulated expression with delay times up to an hr. The hypothesis that liver associated DNA, and not the DNA in blood, is responsible for stimulated expression is deduced by comparison of the level of stimulated expression from PEGylated (Acr-Lys)₆ polyplexes (SEQ ID NO: 33) (FIG. 29B), which decrease by nearly 100-fold between 60 and 120 min while the level of intact DNA in the blood remains constant over that time period (FIG. 30F).

In conclusion, this is the first report of high-level stimulated gene expression from a nonviral delivery system that mirrors the level of expression produced by the same dose administered by direct hydrodynamic dosing. The unique attributes of PEGylated polyacridine peptides establish their ability to form open or closed polyplex structures that stabilize DNA from metabolism in mice and allow a stimulation of a high volume rapid dose of saline to complete the gene transfer, even after a 1 hour delay following the primary dose. This is still only a starting point toward the development of a nonviral delivery system that produces high level expression in animals without stimulation. However, given the modularity of polyacridine peptides, it should be possible to build multi-component gene delivery systems that drive the DNA further toward the nucleus, to ultimately achieve this aim.

### Example 5

### DNA Nanoparticles with Phospholipase A2

Gene therapy has been a goal of medicine for nearly 30 years. Current gene therapy research delivers DNA to tissues through the use of viruses, which have evolved their DNA delivery capabilities over millions of years. Viral gene delivery carries several risks, including immune response. These risks create an interest in nonviral gene delivery methods, however, nonviral techniques have long suffered from low efficiency in vivo compared to viral methods. It is believed that borrowing features from viruses may help to improve gene transfer efficiency. One such feature is phospholipase A2 activity seen in the capsid proteins of several viruses. When the PLA2 activity is removed, the virus becomes far less infective, implying PLA2 activity is important for delivering the viral genome into the host cell. We have developed a novel DNA nanoparticle incorporating bee venom PLA2 for gene delivery. PLA2 was attached to DNA using (Acr-Lys)6-Cys (SEQ ID NO: 92), a polyintercalating peptide previously developed by our lab. (Acr-Lys)6-Cys (SEQ ID NO: 92) was biotinylated on the c-terminal cysteine using a maleimide-biotin. PLA2 was randomly biotinylated on its lysines using a biotin NHS ester. The peptide and enzyme were then bound to avidin and DNA. We have confirmed that PLA2 is bound to DNA through an agarose gel assay and a PLA2 activity assay. This method is applicable to a wide variety of macromolecules, without covalently linking them to DNA, potentially creating a general system for anchoring many functionalities to DNA, without permanently damaging the DNA or hindering genetic processing. This ability could allow for efficient nonviral DNA delivery and gene therapy for the treatment of hereditary disorders, cancers, and other medical issues. A new compound has been generated that is a biotinylated polyacridine peptide that binds to DNA and also binds to Avidin. This compound is used to bind a biotinylated-enzyme (e.g., phospholipase A2, PLA2) onto DNA.

### Example 6

### Formation of Polyplexes of PEGylated Polyacridine Peptides and RNA

The inventors have established that PEGylated polyacridine peptides protect siRNA in the circulation. Atomic force microscopy (AFM) has provided direct evidence that PEGylated polyacridine peptides bind to siRNA to form polyplexes. Polyplex formation is essential to the formation of multi-component siRNA PEGylated PolyAcridine Peptides (PPAPs), since it is otherwise unlikely that more than two or three (Acr-Lys)₆ (SEQ ID NO: 101) peptides will bind to a single siRNA of 22 bp. To achieve polyplex formation, (Acr-Lys)₆ (SEQ ID NO: 101) would have to cross-link individual siRNA molecules. Not only would siRNA PPAPs form via cross-linking, but they would also need to be stable in the circulation. Several lines of evidence suggest this is the case. Binding of PEGylated (Acr-Lys)₆ (SEQ ID NO: 33) caused a band shift of ¹²⁵I-siRNA on PAGE. The formation of siRNA PPAPs with an average size of 50 nm diameter was determined by QELS and confirmed by AFM on anionic mica.

We have also now examined the pharmacokinetics of siRNA polyplexes in mice and have evidence that siRNA is protected in blood for up to 60 minutes. Pharmacokinetic analysis was used to determine if PEGylated (Acr-Lys)₆ siRNA PPAPs (SEQ ID NO: 33) are stable in the circulation. We iodinated an all natural siRNA (22 bp) obtained from Dharmacon. The results established similar a-half-life for PEGylated (Acr-Lys)₆ ¹²⁵I-siRNA PPAPs (SEQ ID NO: 33) compared to ¹²⁵I-siRNA, but a much greater O-half-life, suggesting that PEGylated (Acr-Lys)₆ siRNA PPAPs (SEQ ID NO: 33) are stable and circulate. Separation of blood time points by PAGE followed by detection by autoradiography indicate the siRNA is partially intact in blood for 60 min. We interpret the PAGE result as a composite of both RNA fragments excreted from liver and stable siRNA in blood. The results demonstrate the formation of siRNA PPAPs that have a long circulation in the blood.

### Example 7

### Formation of Man9-(Acr-Lys)₆ (SEQ ID NO: 42)

We tested Man9-N-glycan polyacridine peptides as a DNA vaccine adjuvant for use in combination with an intramuscular electroporation apparatus. We generated data that demonstrated that we could bind up to 40 copies of Man9-(Acr-Lys)₆ (SEQ ID NO: 42) to plasmid (as evidenced by DNA band shift assay), and preserve full gene expression by intramuscular electroporation. The Man9-(Acr-Lys)₆ (SEQ ID NO: 42) that we used for this study has the following structure of In certain embodiments the compound is Formula IVb:

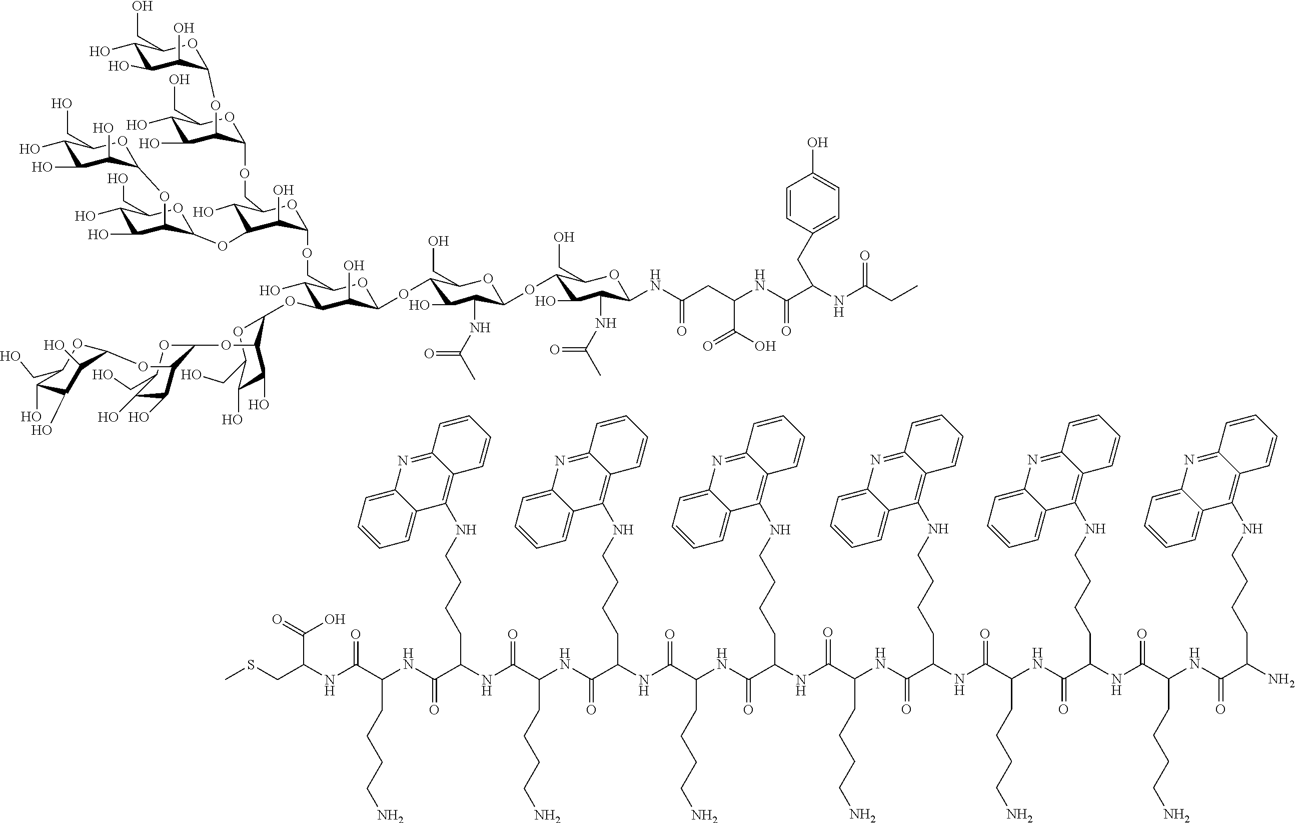

The Man9 N-glycan is derived from the pronase digestion of purified soy bean agglutinin (SBA). The Asn residue on Man9 was first modified by attaching Boc-Tyr the amine terminus followed by removal of Boc. The Tyr amine terminus was then derivatized with iodoacetic acid. The Iodoacetamide Man9 was then reacted Cys(Acr-Lys)₆ (SEQ ID NO: 102) to form the product Man9-(Acr-Lys)₆ (SEQ ID NO: 42).

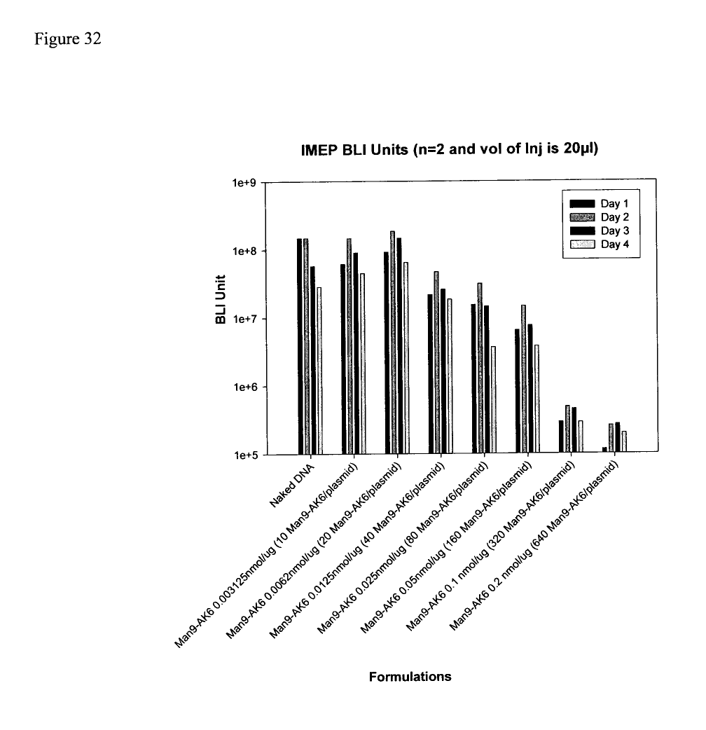

The inventors obtained mouse data illustrating the electroporation apparatus expression of Man9-(Acr-Lys)₆ (SEQ ID NO: 42) DNA (1 μg) at 0 and 0.012 nmoles of Man9-(Acr-Lys)₆ (SEQ ID NO: 42) per μg of DNA (FIG. 32). The luciferase gene expression was equivalent at 0, 0.003 and 0.006 nmols of Man9 per μg of DNA, and then decreased at 0.0125 nmols of Man9 and higher. 0.0125 nmols of Man9-(Acr-Lys)₆ (SEQ ID NO: 42) per μg of DNA corresponds 40 copies of Man9 per plasmid.

### Example 8

Polyacridinylated peptides, which can be used in non-viral gene delivery, provide a reversible means of association with DNA via intercalation of base pairs, but limitations exist in regard to the size of a peptide synthesized via solid phase peptide synthesis (SPPS). Cysteine-flanked, acridinylated peptides were developed. Cysteines at the N- and C-termini of the peptide provide the additional feature of polymerization through disulfide bonding, and the affinity of the polymer for DNA will increase with greater incorporation of monomer. Disulfide bonding is also an attractive feature due to its ability to be reduced in the reductive intracellular environment.

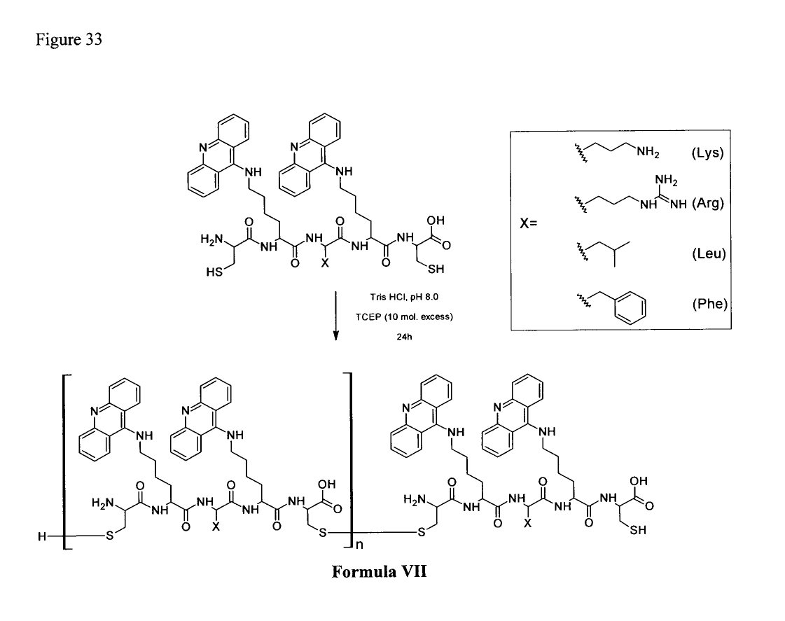

CK_{acr}KK_{acr}C (SEQ ID NO: 103), CK_{acr}RK_{acr}C (SEQ ID NO: 104), CK_{acr}LK_{acr}C (SEQ ID NO: 105), and CK_{acr}FK_{acr}C (SEQ ID NO: 106) (where K_{acr} is a lysine modified with acridine at the ε-amine) (FIG. 33, Formula VII) were synthesized by SPPS and purified while monitoring the absorbance of acridine at 409 nm. Monomers were evaluated for DNA affinity by DNA band shift and thiazole orange displacement assays. DNA complexes with monomers were also evaluated for particle size and ζ-potential. Predictably, positively-charged CK_{acr}KK_{acr}C (SEQ ID NO: 103) and CK_{acr}RK_{acr}C (SEQ ID NO: 104) exhibited higher affinity for DNA. Buffered incubations of monomer at pH 8.0 were used to encourage polymerization of peptides and poly-CK_{acr}KK_{acr}C (SEQ ID NO: 103) and poly-CK_{acr}RK_{acr}C (SEQ ID NO: 104) have been isolated. DNA band shift assays of CK_{acr}KK_{acr}C (SEQ ID NO: 103) monomer and poly-CK_{acr}KK_{acr}C (SEQ ID NO: 103) showed greater affinity of the polymer for DNA than the monomer, but flocculation of the DNA-polymer complex was encountered.

To overcome complex insolubility, similar peptides conjugated via maleimide linkage to poly(ethylene glycol) (i.e., CKK_{acr}C_{mal-mPEG}K_{acr}KC (SEQ ID NO: 43)) are made. Exemplary compounds include the following:

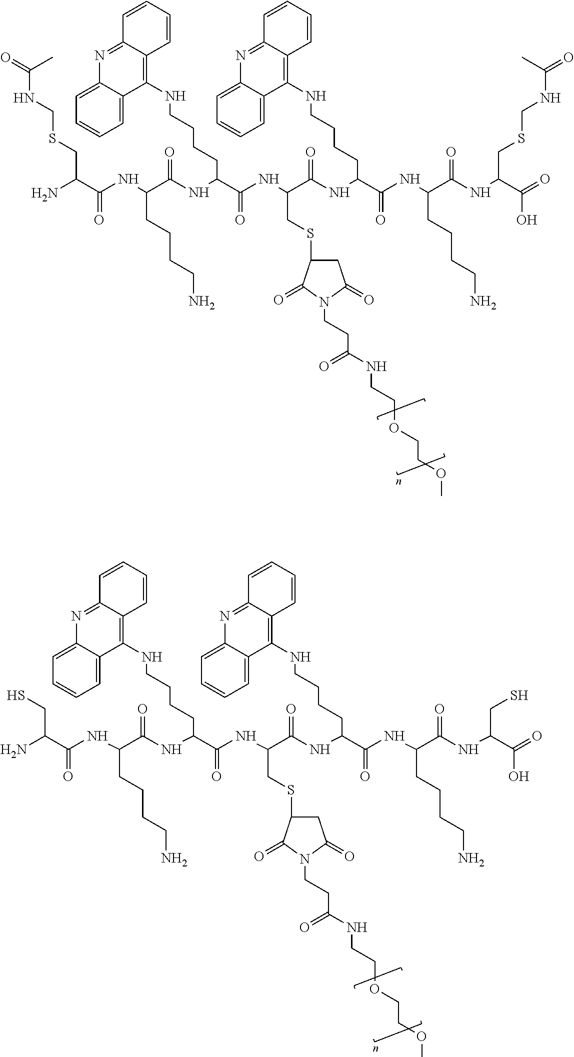

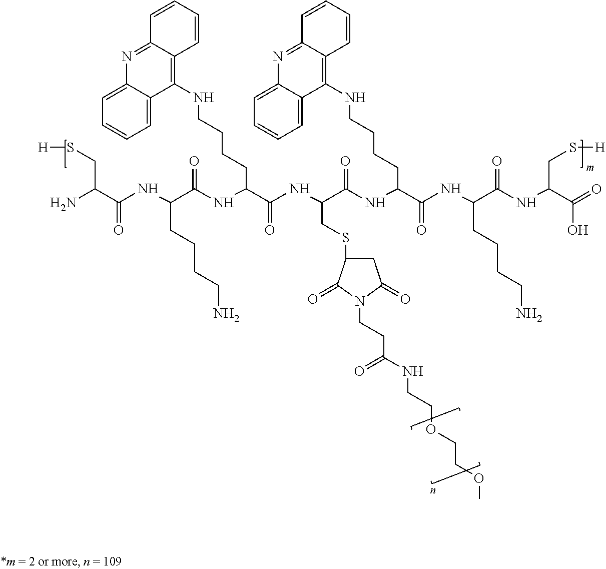

These peptides were made by solid phase peptide synthesis using an orthogonal protection scheme for Cys reported by Park et al. that allowed selective derivatization of the central Cys residue with PEG-maleimide. (Park, Y., Kwok, K. Y., Boukarim, C., and Rice, K. G., “Synthesis of Sulfhydryl Cross-Linking Poly(ethylene glycol)-Peptides and Glycopeptides as Carriers for Gene Delivery.” Bioconj. Chem. (2002) 13, 232-239)

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A nucleic acid delivery peptide complex, comprising 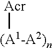
operably linked to
Cys-L¹-R¹
wherein Acr is an acridine; A¹ is Lysine, Arginine, Leucine or Glutamic acid; A² is 1 to 6 amino acids; Cys is cysteine, wherein the Cys is bound to L¹ at the amino-terminus, the carboxy-terminus or an amino acid side-chain; and n is 1 to 10;
wherein L¹ is a linker;
wherein R¹ is one or more of a peptide, polyethylene glycol (PEG), a carbohydrate, biotin or avidin, and/or is a hydrogen; and
wherein the nucleic acid delivery peptide complex reversibly binds to nucleic acid.

2. The nucleic acid delivery peptide complex of claim 1, wherein L is —S—; a disulfide linkage; a maleimide linkage; an amide linkage, ester linkage, ether linkage, or a thiol ether linkage, hydrazone linkage.

3. The nucleic acid delivery peptide complex of claim 1, wherein R¹ is a carbohydrate that is a targeting ligand.

4. The nucleic acid delivery peptide complex of claim 1, wherein R¹ is a carbohydrate that is Man₉GlcNAc₂.

5. The nucleic acid delivery peptide complex of claim 1, wherein R¹ is a peptide that is a nuclear localization signal (NLS) peptide, a fusogenic peptide, a targeting peptide, an NLS-fusogenic peptide or an enzyme.

6. The nucleic acid delivery peptide complex of claim 1, wherein R¹ is polyethylene glycol (PEG) of between 500 g/mol and 20,000 g/mol.

7. The nucleic acid delivery peptide complex of claim 1, wherein A¹ is Lys having an □-amine, and Acr is linked to the □-amine of Lys.

8. The nucleic acid delivery peptide complex of claim 1, wherein A² is one or more of Arg, Lys, Glu or Leu.

9. The nucleic acid delivery peptide complex of claim 1, wherein the Acr is 9-aminoacridine is 9-alkylaminoacridine, 9-arylaminoacridine, 9-(N-carboxamido) acridine or 9-ureidoacridine.

10. A nucleic acid delivery peptide complex, comprising Formula II: 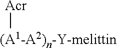
wherein
Acr is acridine;
Y is —S—S— or maleimide;
A¹ is Arginine, Leucine or Lysine;
A² is Arginine, Leucine or Lysine;
n is 2 to 6;
wherein the nucleic acid delivery peptide complex binds to nucleic acid.

11. A nucleic acid delivery peptide complex, comprising Formula III: 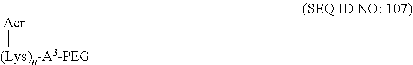
wherein
Acr is acridine;
A³ is Cys or Trp-Cys; and
n is 1 to 6;
wherein the nucleic acid delivery peptide complex binds to nucleic acid.

12. A nucleic acid delivery peptide complex, comprising Formula IV:
(SEQ ID NO: 2)
Man9-L³-(Acr-Lys)ₙ
wherein Man9 is a carbohydrate consisting of nine manose moieties; L³ is a linker, and n is 4-6.

13. The nucleic acid delivery peptide complex of claim 12, wherein the nucleic acid delivery peptide complex comprises Formula IVa:

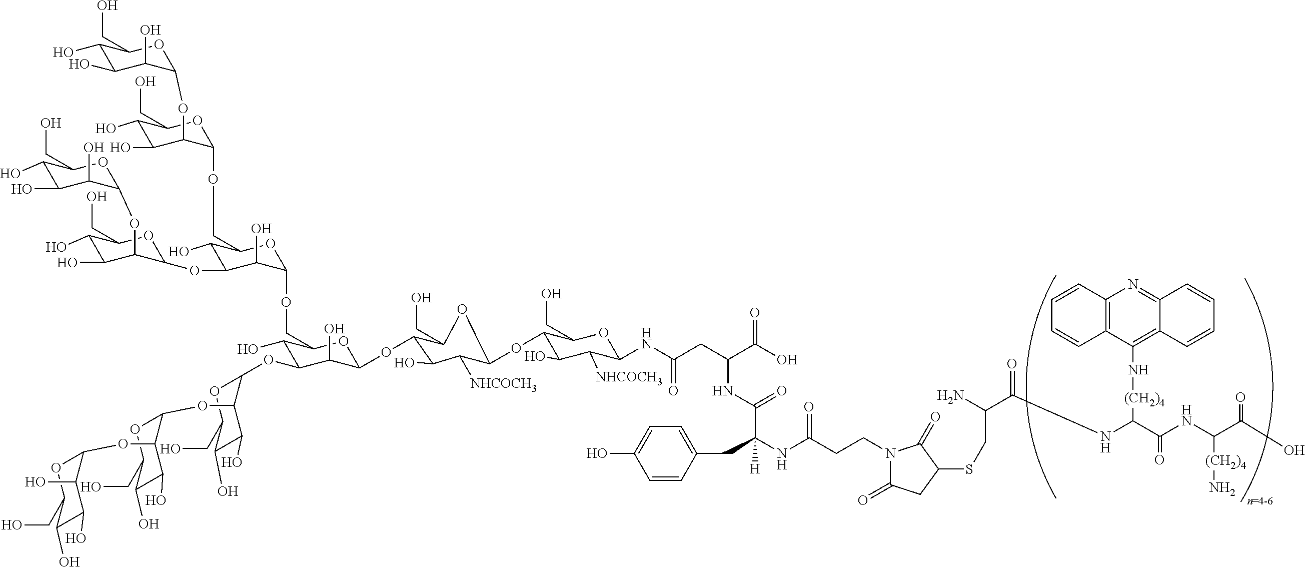

14. The nucleic acid delivery peptide complex of claim 12, wherein the nucleic acid delivery peptide complex comprises Formula IVb:

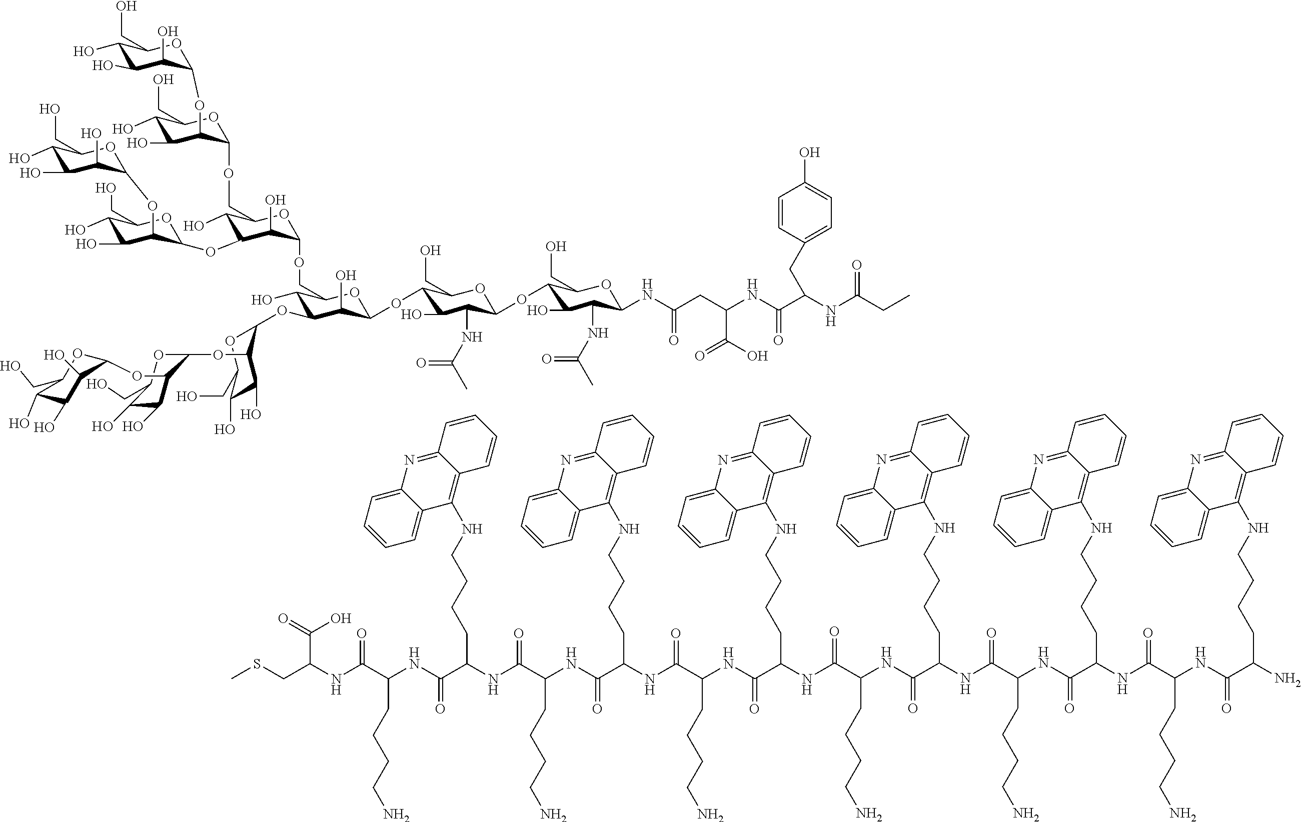

15. A nucleic acid delivery peptide complex of claim 1, comprising Formula Ia, Ib, Ic or Id: 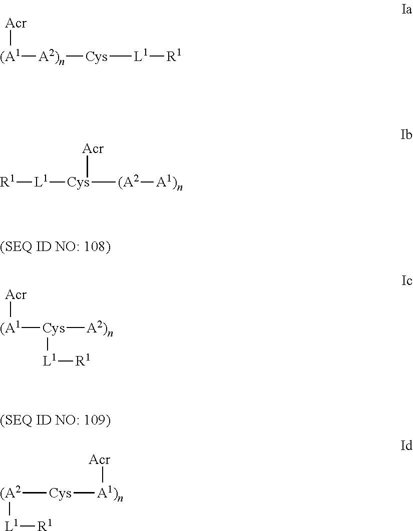
wherein Acr is an acridine; A¹ is Lysine, Arginine, Leucine or Glutamic acid; A² is 1 to 6 amino acids; Cys is cysteine, wherein the Cys is bound to Y at the amino-terminus, the carboxy-terminus or an amino acid side-chain; and n is 1 to 10;
wherein L¹ is a linker;
wherein R¹ is one or more of a peptide, polyethylene glycol (PEG), a carbohydrate, and/or is a hydrogen; and
wherein the nucleic acid delivery peptide complex reversibly binds to nucleic acid.

16. The nucleic acid delivery peptide complex of claim 10, wherein the complex is poly(Acr-Arg)₄-SS-melittin (SEQ ID NO: 78).

17. A nucleic acid delivery complex comprising a nucleic acid molecule reversibly bound to the peptide complex of claim 1.

18. The nucleic acid delivery complex of claim 17, wherein the nucleic acid is an RNA molecule.

19. An anionic open polyplex comprising a nucleic acid molecule reversibly bound to the peptide complex of claim 1, wherein the anionic open polyplex is stable to digestion with 60 milliunits of DNase or RNase digestions for 60 minutes.

20. A nucleic acid delivery peptide complex comprising:
(a) a compound of Formula V: 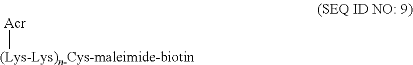
wherein n is 4-6;
(b) a biotinylated phospholipase A2; and
(c) avidin operably linked to both the compound of Formula V and the biotinylated phospholipase A2;
wherein the nucleic acid delivery peptide complex reversibly binds to nucleic acid.

21. A nucleic acid delivery peptide complex, comprising
(SEQ ID NO: 10)
(Cys-Y-Lys_{acr}-Z-Lys_{acr}-Y-Cys)ₙ
operably linked to -L¹-R¹;
wherein Lys_{acr} is a lysine modified with acridine at the s-amine; Cys is Cysteine; Lys is Lysine; Y is Lys or a covalent bond; Z is Lysine, Arginine, Leucine, Phenylalanine, or Cysteine, wherein Z is optionally conjugated by a maleimide linkage to poly(ethylene glycol);
n is 1 to 30;
L¹ is a linker and is bound to Cys at the amino-terminus, the carboxy-terminus or an amino acid side-chain;
R¹ is one or more of a peptide, polyethylene glycol (PEG), a carbohydrate, and/or is a hydrogen; and
wherein the nucleic acid delivery peptide reversibly binds to nucleic acid.
